(19) United States
(12) Patent Application Publication
(10) Pub. No.: US 2016/0235839 A1
(43) Pub. Date: Aug. 18, 2016

(54) CALCIUM FLUORIDE COMPOSITIONS

(71) Applicant: GlaxoSmithKline Biologicals, S.A., Rixensart (BE)

(72) Inventor: Vincent Vande Velde, Rixensart (BE)

(73) Assignee: GlaxoSmithKline Biologicals, S.A., Rixensart (BE)

(21) Appl. No.: 15/031,338

(22) PCT Filed: Oct. 23, 2014
(86) PCT No.: PCT/EP2014/072740

§ 371 Date: Apr. 22, 2016

Related U.S. Application Data
(60) Provisional application No. 61/946,040, filed on Feb. 28, 2014.

(30) Foreign Application Priority Data
Oct. 25, 2013 (GB) 1318862.8

(51) Int. Cl. A61K 39/39; C12N 7/00; A61K 39/155; A61K 39/29

(52) U.S. Cl. CPC A61K 39/39; A61K 39/292; C12N 7/00; A61K 39/155; A61K 2039/55505

(57) ABSTRACT

Compositions comprising calcium fluoride composites comprising Ca, F, and an organic molecule are provided, as are methods for their use.

### TECHNICAL FIELD

The present disclosure relates to composites for the stabilization of vaccine antigens and for enhancing the immune response against antigens used with the composites.

### BACKGROUND

Subunit vaccines, for example recombinant protein/polypeptide antigens are only weakly immunogenic and thus there is a need for safe and effective adjuvants. Various adjuvants are known, including those comprising metallic salts such as alum, aluminum phosphate, and calcium phosphate. See, e.g., Lindblad (2004) Vaccine 22:3658-3668; Jiang et al (2004) Vaccine 23:693-698.

Thermo-stability of vaccines is desirable for practical and logistic reasons as thermostability of the vaccine reduces or avoids the requirement for cold-chain during worldwide distribution. Usually, lyophilisation techniques are applied to stabilize antigens. However, lyophilisation is not always possible or effective. Moreover, bypassing the costly and time consuming lyophilisation production step could increase the accessibility of the vaccine to a larger number of people in the world.

### SUMMARY OF THE INVENTION

In one aspect, this disclosure provides calcium fluoride composites comprising Ca, F, and Z, wherein Z is an organic molecule. Methods for their production are provided. Methods for their use as adjuvants are also provided, as are methods for their use to stabilize antigens against temperature effects. Such methods include the use of some composites without lyophilization.

In a further aspect are provided calcium fluoride compositions comprising a calcium fluoride composite, said composite comprising Ca, F, and Z, wherein Z is an organic molecule.

In a further aspect, are provided processes for making a calcium fluoride composite by sol gel precipitation comprising the steps of combining CaCl2, NaF, and NaZ under precipitating conditions and collecting the water insoluble calcium fluoride composite. In a further aspect are provided products made by the process.

In a further aspect are provided adjuvant compositions comprising a calcium fluoride composition disclosed in the preceding aspects.

In a further aspect are provided processes for making the adjuvant compositions disclosed in the preceding aspects.

In a further aspect are provided immunogenic compositions comprising an antigen and an adjuvant composition as disclosed in the preceding aspects.

In a further aspect are provided processes for making immunogenic compositions as disclosed in the preceding aspects.

### BRIEF DESCRIPTION OF THE FIGURES

FIG. 1: Animal results obtained with HepB: antibody measurements (anti-HBs 14pII). Responses of the antigen are maintained when the antigen is adsorbed on the different carriers of the CaF₂ family described herein.

FIG. 2: Infrared spectra of batches 8833107 compared to 8833111. The infrared analysis shows the presence of CaCO₃ of the Vaterite type.

FIG. 3: Water solubility of Ca/F/OH composite, revealing that the composite is more soluble compared to the solubility of CaF₂ reported in handbooks.

FIG. 4: F4T formulation analyzed by SDS-PAGE analyses at t=0. Lane: 1, molecular weight standard; 2, sample buffer; 3, CaF₂/CO₃+liposome; 4, F4T+CaF₂; 5, F4T+CaF₂+liposome; 6, F4T+CaF₂/cysteine; 7, F4T+CaF₂/cysteine+liposome; 8, F4T+CaF₂/CO₃; 9, F4T+CaF₂/CO₃+liposome; 10, F4T. See Example 3B.

FIG. 5: F4T formulation analyzed by SDS-PAGE after 1 month at 4° C. Lane: 1, molecular weight standard; 2, F4T bulk: bulk stored 1 month at −80 C and thawed just before depot; 3, F4T bulk stored 1 month at 4 C; 4, F4T formulated without inorganic and stored 1 month at 4 C; 5, F4T+CaF₂; 6, F4T+CaF₂+liposome; 7, F4T+CaF₂/cysteine; 8, F4T+CaF₂/cysteine+liposome; 9, F4T+CaF₂/CO₃; 10, F4T+CaF₂/CO₃+liposome. See Example 3B.

FIG. 6: F4T formulations analyzed by SDS-PAGE after 1 month at 30° C. Lane: 1, molecular weight standard; 2, F4T bulk: bulk stored 1 month at −80 C and thawed just before depot; 3, F4T bulk stored 1 month at 30 C; 4, F4T formulated without inorganic and stored 1 month at 30 C; 5, F4T+CaF₂; 6, F4T+CaF₂+liposome; 7, F4T+CaF₂/cysteine; 8, F4T+CaF₂/cysteine+liposome; 9, F4T+CaF₂/CO₃; 10, F4T+CaF₂/CO₃+liposome. Note the substantial degradation of lanes 3 and 4 (F4T without composite). See Example 3B.

FIG. 7: Composite+ClfA_{N123} immunogenicity (antibodies). The immunogenicity of the antigen is maintained when the antigen is adsorbed on the different carriers. Mice were immunized with stabilized ClfA_{N123} composite (adsorbed on an inorganic carrier). The immunogenicity of these adsorbed composite in an emulsion formulation was carried out by ELISA-ClfA_{N123} composite (concentrations (μg/mL) on Post III. From left to right, non-treated, adsorbed on CaF₂/CaCO₃, adsorbed on CaF₂/N—Ac-Cysteine, adsorbed on CaF₂, and adsorbed on CaF₂/Cysteine. See Example 4.

FIG. 8: Infrared spectra of batches 8833152-7.

FIG. 9: Immune Response of HepB adsorbed antigen. See Example 5.

FIG. 10: Electron Microscopy photograph of calcium fluoride composites disclosed herein. Pictured are calcium fluoride composites disclosed in batch #10616125 (see Table 1 and the example entitled “Ca/F/N-Acetyl-cysteine batch #10616125.”

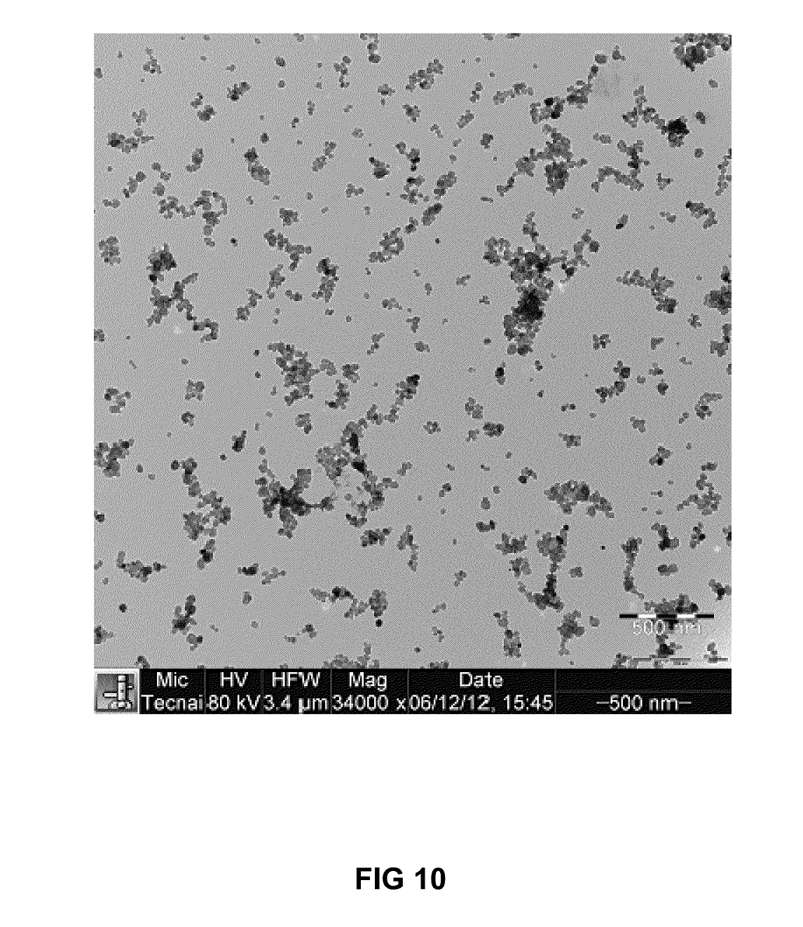

FIG. 11: RSV neutralization titers in serum 14 days after the second immunization with rF antigen at two different doses adsorbed on different composites. See Example 6.

FIG. 12: Anti-rF IgG concentrations in serum 14 days after the second immunization with rF antigen at two different doses adsorbed on different composites. See Example 6.

FIG. 13: RSV titers in lungs 4 days after RSV challenge, according to various regimens composed of 2 μg rF and adjuvant. See Example 7.

FIG. 14: Evaluation of composite-19F-DT formulations in the Balb/c mouse immunogenicity model. See Example 8.

FIG. 15: Evaluation of composite-19F-DT formulations in the Balb/c mouse immunogenicity model (cont). See Example 8.

FIG. 16: Evaluation of composite-19F-DT formulations in the Balb/c mouse immunogenicity model (cont). See Example 8.

FIG. 17: Evaluation of composite-19F-DT formulations in the Balb/c mouse immunogenicity model (cont). See Example 8.

FIG. 18: Animal Results of various composite-PRN. See Example 9.

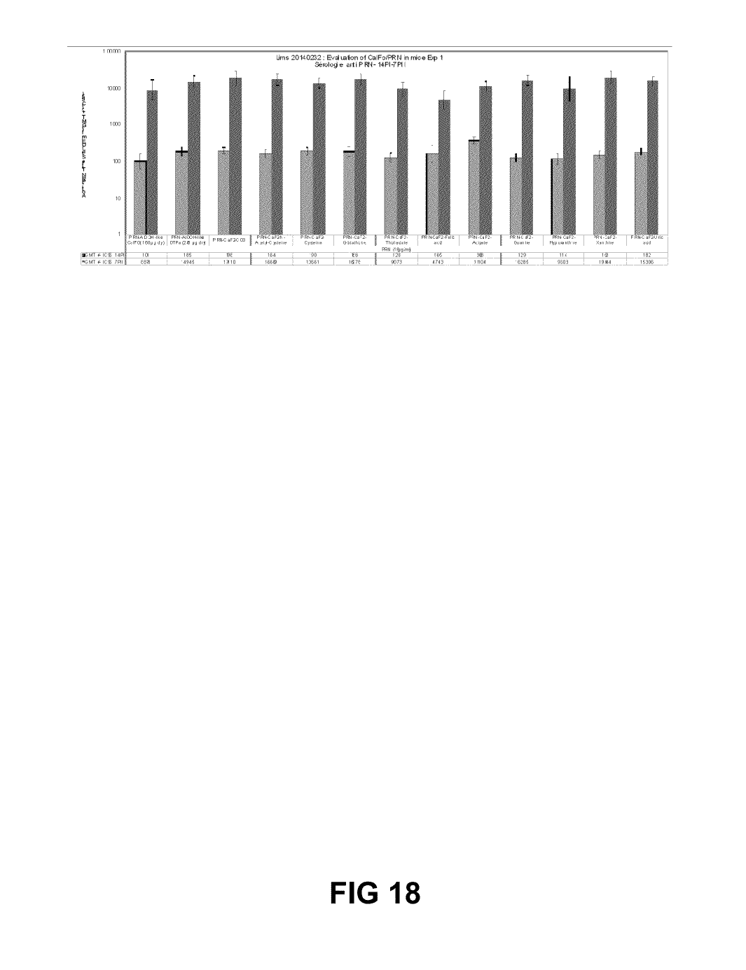

### DETAILED DESCRIPTION

It is disclosed herein that adsorption of antigens to a water insoluble a calcium fluoride composite stabilizes the antigen against temperature dependent degradation. Moreover, it is disclosed that the calcium fluoride composites act as an adjuvant by increasing the immune response against an antigen adsorbed thereto.

### Compositions

In some aspects are provided calcium fluoride compositions comprising a calcium fluoride composite, the composite comprising Ca, F, and Z. By “Z” is intended an organic (carbon-containing) molecule. By “composite” is intended a material that exists as a solid when dry, and that is insoluble, or poorly soluble, in pure water.

In some aspects, the composite comprises equal percentages w/w of Ca and F. In some aspects, the composite comprises a greater percentage Ca (w/w) than percentage F (w/w). By “percent X w/w” (where X is a molecule or element found in a composition) is intended the percentage of the total weight of the composition that is attributable to X. Thus, w/w in the present context means the dry weight. For compositions in which the relative ratios are known, the percent w/w may be determined mathematically. For instance, compositions of CaF₂ comprise roughly 51% Ca and roughly 49% F (w.w): % w/w Ca=[(40 g/mol Ca*100)]/[40 g/mol Ca+(2*19 g/mol F)]=51; % w/w F=[(2*19 g/mol F)*100]/[40 g/mol Ca+(2*19 g/mol F)]=49. Nonetheless, % w/w may also be determined by empirical methods. For instance, where the molecule in question is an acid or base, the percent w/w of that molecule may be determined by titration (where Z is carbonate, percent w/w/carbonate can be determined by titration with HCl). Alternatively, where the molecule contains a fractional percent by weight of nitrogen, the percent w/w of that molecule may be determined by elementary analysis methods in which the amount of nitrogen is determined and then the total weight attributable to the nitrogen-containing molecule calculated using the molecular weight of the nitrogen-containing molecule. Instruments for this methodology are available commercially, for instance from Antek™, 300 Bammel Westfield Road, Houston, Tex. 77090. Alternatively, percent w/w of an oxidizable organic molecule can be determined by oxydo-reduction titration methods, for example in the presence of potassium permanganate in the presence of sulfuric acid.

In some aspects, calcium fluoride composites as disclosed herein may be represented as follows:

CaF₍₂₋ₓ₎Z₍ₓ₎/Z_{(y)} Formula I

where x is a non-negative number from 0 to 2, inclusive, and y is a non-negative number. In some aspects, y is a non-negative number from 0 to 2, inclusive. In some aspects, the sum of x and y together is a non-negative number of equal to or less than 2. In some aspects, x and y are not both zero. However, as will be understood given their formation, a calcium fluoride composite as described herein may not be uniform, but may rather comprise regions in which Z interacts with the rest of the constituents by primarily ionic or covalent interactions and regions in which Z interacts with the rest of the constituents through weak forces (represented by “/Z”). In this context, Z₍ₓ₎ represents the ionized form of Z and Z_{(y)} represents the unionized form of Z, such as HZ or AZ, or a mixture thereof, where A is a counterion. Such non-uniform composites may be represented as follows:

CaF₍₂₋ₓ₎Z₍ₓ₎/HZ_{(y)} Formula II

or

CaF₍₂₋ₓ₎Z₍ₓ₎/AZ_{(y)} Formula III

wherein neither x nor y are both not zero.

Calcium fluoride composites as disclosed herein will have the characteristics of forming a solid when dry, will be insoluble, or poorly soluble, in pure water, and exhibit an E.C.P. in the range of 5.0 to 11.0, inclusive.

In some aspects, Z comprises a functional group that forms an anion when ionized. Such functional groups include without limitation one or more functional groups selected from the group consisting of: hydroxyl, hydroxylate, hydroxo, oxo, N-hydroxylate, hydroaxamate, N-oxide, bicarbonate, carbonate, carboxylate, fatty acid, thiolate, organic phosphate, dihydrogenophosphate, monohydrogenophosphate, monoesters of phosphoric acid, diesters of phosphoric acid, esters of phospholipid, phosphorothioate, sulphates, hydrogen sulphates, enolate, ascorbate, phosphoascorbate, phenolate, and imine-olates. In some aspects, the calcium fluoride composites herein comprise Z, where Z is an anionic organic molecule possessing an affinity for calcium and forming a water insoluble composite with calcium and fluoride.

In some aspects, the calcium fluoride composites herein comprise Z, where Z may be categorized as comprising a member of a chemical category selected from the group consisting of: hydroxyl, hydroxylates, hydroxo, oxo, N-hydroxylate, hydroaxamate, N-oxide, bicarbonates, carbonates, carboxylates and dicarboxylate, salts of carboxylic-acids, salts of QS21, extract of bark of Quillaja saponaria, extract of immunological active saponine, salts of saturated or unsaturated fatty acid, salts of oleic acid, salts of amino-acids, thiolates, thiolactate, salt of thiol-compounds, salts of cysteine, salts of N-acetyl-cysteine, L-2-Oxo-4-thiazolidinecarboxylate, phosphates, dihydrogenophosphates, monohydrogenophosphate, salts of phosphoric-acids, monoesters of phosphoric acids and their salts, diesters of phosphoric acids and their salts, esters of 3-O-desacyl-4′-monophophoryl lipid A, esters of 3D-MLA, MPL, esters of phospholipids, DOPC, dioleolyphosphatidic derivatives, phosphates from CPG motifs, phosphorothioates from CpG family, sulphates, hydrogen sulphates, salts of sulphuric acids, enolates, ascorbates, phosphoascorbate, phenolate, α-tocopherol, imine-olates, cytosine, methyl-cytosine, uracyl, thymine, barbituric acid, hypoxanthine, inosine, guanine, guanosine, 8-oxo-adenine, xanthine, uric acid, pteroic acid, pteroylglutamic acid, folic acid, riboflavin, and lumiflavin.

In some aspects, the calcium fluoride composites herein comprise Z, where Z is selected from the group consisting of: N-acetyl cysteine; thiolactate; adipate; carbonate; folic acid; glutathione; and uric acid. In some aspects, the calcium fluoride composites herein comprise Z, where Z is selected from the group consisting of: N-acetyl cysteine; adipate; carbonate; and folic acid.

In some aspects, the calcium fluoride composites herein comprise Z, where Z is N-acetyl cysteine, and the composite comprises between 51% Ca, 48% F, no more than 1% N-acetyl cysteine (w/w) and 37% Ca, 26% F, and 37% N-acetyl cysteine (w/w).

In some aspects, the calcium fluoride composites herein comprise Z, where Z is Z is thiolactate, and the composite comprises between 51% Ca, 48% F, no more than 1% thiolactate (w/w) and 42% Ca, 30% F, 28% thiolactate (w/w).

In some aspects, the calcium fluoride composites herein comprise Z, where Z is Z is adipate, and the composite comprises between 51% Ca, 48% F, no more than 1% adipate (w/w) and 38% Ca, 27% F, 35% adipate (w/w).

In some aspects, the calcium fluoride composites herein comprise Z, where Z is Z is carbonate, and the composite comprises between 51% Ca, 48% F, no more than 1% carbonate (w/w) and 48% Ca, 34% F, 18% carbonate (w/w).

In some aspects, the calcium fluoride composites herein comprise Z, where Z is Z is folic acid, and the composite comprises between 51% Ca, 48% F, no more than 1% folic acid (w/w) and 22% Ca, 16% F, 62% folic acid (w/w).

In some aspects, the calcium fluoride composites herein comprise Z, where Z is glutathione, and the composite comprises between 51% Ca, 48% F, no more than 1% glutathione (w/w) and 28% Ca, 20% F, 52% glutathione (w/w).

In some aspects, the calcium fluoride composites herein comprise Z, where Z is uric acid, and the composite comprises between 51% Ca, 48% F, and no more than 1% uric acid (w/w) and 36% Ca, 26% F, and 38% uric acid (w/w).

In some aspects, a calcium fluoride composite comprising Ca, F, and Z has the following composition (Chart 1):

CHART 1
Ca/F/Z % W/W calculation for various composites.

| | | Ca[(F₂)100-% + (Z) %], | | | | | | |
|---|---|---|---|---|---|---|---|---|
| based on | | where % is 0, or 2 or 5 or 10 or 15 or 20 or 25 | | | | | | |
| | Z Formula | | | | Mass/ | | | |
| | Weight | | | | mole | | | |
| | (FW) | | | | composite | | | |
| | | | | | | Ca % | F % | NAcCys |
| Ca/F/NAcetylCysteine | | | F₂ | Ca | PM | w/w | w/w | % |
| % | 161.0 | | 38.0 | 40.0 | | | | |
| 0.0 | 0.0 | 100.0 | 38.0 | 40.0 | 78.0 | 51.3 | 48.7 | 0.0 |
| 2.0 | 3.2 | 98.0 | 37.2 | 40.0 | 80.5 | 49.7 | 46.3 | 4.0 |
| 5.0 | 8.1 | 95.0 | 36.1 | 40.0 | 84.2 | 47.5 | 42.9 | 9.6 |
| 10.0 | 16.1 | 90.0 | 34.2 | 40.0 | 90.3 | 44.3 | 37.9 | 17.8 |
| 15.0 | 24.2 | 85.0 | 32.3 | 40.0 | 96.5 | 41.5 | 33.5 | 25.0 |
| 20.0 | 32.2 | 80.0 | 30.4 | 40.0 | 102.6 | 39.0 | 29.6 | 31.4 |
| 25.0 | 40.3 | 75.0 | 28.5 | 40.0 | 108.8 | 36.8 | 26.2 | 37.0 |
| Ca/F/Thiolactate | | | F₂ | Ca | PM | Ca % | F % | Thiolactate % |
| % | 104.0 | | 38.0 | 40.0 | | | | |
| 0.0 | 0.0 | 100.0 | 38.0 | 40.0 | 78.0 | 51.3 | 48.7 | 0.0 |
| 2.0 | 2.1 | 98.0 | 37.2 | 40.0 | 79.3 | 50.4 | 46.9 | 2.6 |
| 5.0 | 5.2 | 95.0 | 36.1 | 40.0 | 81.3 | 49.2 | 44.4 | 6.4 |
| 10.0 | 10.4 | 90.0 | 34.2 | 40.0 | 84.6 | 47.3 | 40.4 | 12.3 |
| 15.0 | 15.6 | 85.0 | 32.3 | 40.0 | 87.9 | 45.5 | 36.7 | 17.7 |
| 20.0 | 20.8 | 80.0 | 30.4 | 40.0 | 91.2 | 43.9 | 33.3 | 22.8 |
| 25.0 | 26.0 | 75.0 | 28.5 | 40.0 | 94.5 | 42.3 | 30.2 | 27.5 |
| Ca/F/Adipate | | | F₂ | Ca | PM | Ca % | F % | Adipate % |
| % | 144.0 | | 38.0 | 40.0 | | | | |
| 0.0 | 0.0 | 100.0 | 38.0 | 40.0 | 78.0 | 51.3 | 48.7 | 0.0 |
| 2.0 | 2.9 | 98.0 | 37.2 | 40.0 | 80.1 | 49.9 | 46.5 | 3.6 |
| 5.0 | 7.2 | 95.0 | 36.1 | 40.0 | 83.3 | 48.0 | 43.3 | 8.6 |
| 10.0 | 14.4 | 90.0 | 34.2 | 40.0 | 88.6 | 45.1 | 38.6 | 16.3 |
| 15.0 | 21.6 | 85.0 | 32.3 | 40.0 | 93.9 | 42.6 | 34.4 | 23.0 |
| 20.0 | 28.8 | 80.0 | 30.4 | 40.0 | 99.2 | 40.3 | 30.6 | 29.0 |
| 25.0 | 36.0 | 75.0 | 28.5 | 40.0 | 104.5 | 38.3 | 27.3 | 34.4 |
| Ca/F/Cysteine | | | F₂ | Ca | PM | Ca % | F % | Cysteine % |
| % | 119.0 | | 38.0 | 40.0 | | | | |
| 0.0 | 0.0 | 100.0 | 38.0 | 40.0 | 78.0 | 51.3 | 48.7 | 0.0 |
| 2.0 | 2.4 | 98.0 | 37.2 | 40.0 | 79.6 | 50.2 | 46.8 | 3.0 |
| 5.0 | 6.0 | 95.0 | 36.1 | 40.0 | 82.1 | 48.8 | 44.0 | 7.3 |
| 10.0 | 11.9 | 90.0 | 34.2 | 40.0 | 86.1 | 46.5 | 39.7 | 13.8 |
| 15.0 | 17.9 | 85.0 | 32.3 | 40.0 | 90.2 | 44.4 | 35.8 | 19.8 |
| 20.0 | 23.8 | 80.0 | 30.4 | 40.0 | 94.2 | 42.5 | 32.3 | 25.3 |
| 25.0 | 29.8 | 75.0 | 28.5 | 40.0 | 98.3 | 40.7 | 29.0 | 30.3 |
| | | | | | | Ca % | F % | Glutathione |
| Ca/F/Glutathione | | | F₂ | Ca | PM | w/w | w/w | % |
| % | 305.0 | | 38.0 | 40.0 | | | | |
| 0.0 | 0.0 | 100.0 | 38.0 | 40.0 | 78.0 | 51.3 | 48.7 | 0.0 |
| 2.0 | 6.1 | 98.0 | 37.2 | 40.0 | 83.3 | 48.0 | 44.7 | 7.3 |
| 5.0 | 15.3 | 95.0 | 36.1 | 40.0 | 91.4 | 43.8 | 39.5 | 16.7 |
| 10.0 | 30.5 | 90.0 | 34.2 | 40.0 | 104.7 | 38.2 | 32.7 | 29.1 |
| 15.0 | 45.8 | 85.0 | 32.3 | 40.0 | 118.1 | 33.9 | 27.4 | 38.8 |
| 20.0 | 61.0 | 80.0 | 30.4 | 40.0 | 131.4 | 30.4 | 23.1 | 46.4 |
| 25.0 | 76.3 | 75.0 | 28.5 | 40.0 | 144.8 | 27.6 | 19.7 | 52.7 |
| | | | | | | Ca % | F % | Glutathione |
| Ca/F/Glutathione oxide | | | F₂ | Ca | PM | w/w | w/w | oxide % |
| % | 610.0 | | 38.0 | 40.0 | | | | |
| 0.0 | 0.0 | 100.0 | 38.0 | 40.0 | 78.0 | 51.3 | 48.7 | 0.0 |
| 2.0 | 12.2 | 98.0 | 37.2 | 40.0 | 89.4 | 44.7 | 41.6 | 13.6 |
| 5.0 | 30.5 | 95.0 | 36.1 | 40.0 | 106.6 | 37.5 | 33.9 | 28.6 |
| 10.0 | 61.0 | 90.0 | 34.2 | 40.0 | 135.2 | 29.6 | 25.3 | 45.1 |
| 15.0 | 91.5 | 85.0 | 32.3 | 40.0 | 163.8 | 24.4 | 19.7 | 55.9 |
| 20.0 | 122.0 | 80.0 | 30.4 | 40.0 | 192.4 | 20.8 | 15.8 | 63.4 |
| 25.0 | 152.5 | 75.0 | 28.5 | 40.0 | 221.0 | 18.1 | 12.9 | 69.0 |
| | | | | | | Ca % | F % | Uric acid |
| Ca/F/Uric acid | | | F₂ | Ca | PM | w/w | w/w | % |
| % | 166.0 | | 38.0 | 40.0 | | | | |
| 0.0 | 0.0 | 100.0 | 38.0 | 40.0 | 78.0 | 51.3 | 48.7 | 0.0 |
| 2.0 | 3.3 | 98.0 | 37.2 | 40.0 | 80.6 | 49.7 | 46.2 | 4.1 |
| 5.0 | 8.3 | 95.0 | 36.1 | 40.0 | 84.4 | 47.4 | 42.8 | 9.8 |
| 10.0 | 16.6 | 90.0 | 34.2 | 40.0 | 90.8 | 44.1 | 37.7 | 18.3 |
| 15.0 | 24.9 | 85.0 | 32.3 | 40.0 | 97.2 | 41.2 | 33.2 | 25.6 |
| 20.0 | 33.2 | 80.0 | 30.4 | 40.0 | 103.6 | 38.6 | 29.3 | 32.0 |
| 25.0 | 41.5 | 75.0 | 28.5 | 40.0 | 110.0 | 36.4 | 25.9 | 37.7 |
| | | | | | | Ca % | F % | Folic acid |
| Ca/F/Folic acid | | | F₂ | Ca | PM | w/w | w/w | % |
| % | 439.0 | | 38.0 | 40.0 | | | | |
| 0.0 | 0.0 | 100.0 | 38.0 | 40.0 | 78.0 | 51.3 | 48.7 | 0.0 |
| 2.0 | 8.8 | 98.0 | 37.2 | 40.0 | 86.0 | 46.5 | 43.3 | 10.2 |
| 5.0 | 22.0 | 95.0 | 36.1 | 40.0 | 98.1 | 40.8 | 36.8 | 22.4 |
| 10.0 | 43.9 | 90.0 | 34.2 | 40.0 | 118.1 | 33.9 | 29.0 | 37.2 |
| 15.0 | 65.9 | 85.0 | 32.3 | 40.0 | 138.2 | 29.0 | 23.4 | 47.7 |
| 20.0 | 87.8 | 80.0 | 30.4 | 40.0 | 158.2 | 25.3 | 19.2 | 55.5 |
| 25.0 | 109.8 | 75.0 | 28.5 | 40.0 | 178.3 | 22.4 | 16.0 | 61.6 |
| | | | | | | Ca % | F % | Folic acid |
| Ca/F/CO3 | | | F₂ | Ca | PM | w/w | w/w | % |
| % | 60.0 | | 38.0 | 40.0 | | | | |
| 0.0 | 0.0 | 100.0 | 38.0 | 40.0 | 78.0 | 51.3 | 48.7 | 0.0 |
| 2.0 | 1.2 | 98.0 | 37.2 | 40.0 | 78.4 | 51.0 | 47.5 | 1.5 |
| 5.0 | 3.0 | 95.0 | 36.1 | 40.0 | 79.1 | 50.6 | 45.6 | 3.8 |
| 10.0 | 6.0 | 90.0 | 34.2 | 40.0 | 80.2 | 49.9 | 42.6 | 7.5 |
| 15.0 | 9.0 | 85.0 | 32.3 | 40.0 | 81.3 | 49.2 | 39.7 | 11.1 |
| 20.0 | 12.0 | 80.0 | 30.4 | 40.0 | 82.4 | 48.5 | 36.9 | 14.6 |
| 25.0 | 15.0 | 75.0 | 28.5 | 40.0 | 83.5 | 47.9 | 34.1 | 18.0 |
| | | | | | | Ca | F % | Hypo- |
| Ca/F/Hypoxanthine | | | F₂ | Ca | PM | % w/w | w/w | xanthine % |
| % | 135 | | 38.0 | 40.0 | | | | |
| 0.0 | 0.0 | 100.0 | 38.0 | 40.0 | 78.0 | 51.3 | 48.7 | 0.0 |
| 2.0 | 2.7 | 98.0 | 37.2 | 40.0 | 79.9 | 50.0 | 46.5 | 3.3 |
| 5.0 | 6.75 | 95.0 | 36.1 | 40.0 | 82.85 | 48.2 | 43.5 | 8.1 |
| 10.0 | 13.5 | 90.0 | 34.2 | 40.0 | 87.7 | 45.61 | 38.9 | 15.4 |
| 15.0 | 20.25 | 85.0 | 32.3 | 40.0 | 92.55 | 43.2 | 34.9 | 21.8 |
| 20.0 | 27.0 | 80.0 | 30.4 | 40.0 | 97.4 | 41.0 | 31.2 | 27.7 |
| 25.0 | 33.75 | 75.0 | 28.5 | 40.0 | 102.25 | 39.1 | 27.8 | 33.0 |
| | | | | | | Ca % | F % | Xanthine |
| Ca/F/Xanthine | | | F₂ | Ca | PM | w/w | w/w | % |
| % | 151 | | 38.0 | 40.0 | | | | |
| 0.0 | 0.0 | 100.0 | 38.0 | 40.0 | 78.0 | 51.3 | 48.7 | 0.0 |
| 2.0 | 3.0 | 98.0 | 37.2 | 40.0 | 80.2 | 49.86 | 46.3 | 3.7 |
| 5.0 | 7.5 | 95.0 | 36.1 | 40.0 | 83.6 | 47.82 | 43.1 | 9.0 |
| 10.0 | 15.1 | 90.0 | 34.2 | 40.0 | 89.3 | 44.79 | 38.3 | 16.9 |
| 15.0 | 22.6 | 85.0 | 32.3 | 40.0 | 94.9 | 42.13 | 34.0 | 23.8 |
| 20.0 | 30.2 | 80.0 | 30.4 | 40.0 | 100.6 | 39.76 | 30.2 | 30.0 |
| 25.0 | 37.7 | 75.0 | 28.5 | 40.0 | 106.2 | 37.65 | 26.8 | 35.5 |
| | | | | | | Ca % | F % | Guanine |
| Ca/F/Guanine | | | F₂ | Ca | PM | w/w | w/w | % |
| % | 150 | | 38.0 | 40.0 | | | | |
| 0.0 | 0.0 | 100.0 | 38.0 | 40.0 | 78.0 | 51.3 | 48.7 | 0.0 |
| 2.0 | 3.0 | 98.0 | 37.2 | 40.0 | 80.2 | 49.8 | 46.3 | 3.7 |
| 5.0 | 7.5 | 95.0 | 36.1 | 40.0 | 83.6 | 47.8 | 43.1 | 8.9 |
| 10.0 | 15.0 | 90.0 | 34.2 | 40.0 | 89.2 | 44.8 | 38.3 | 16.8 |
| 15.0 | 22.5 | 85.0 | 32.3 | 40.0 | 94.8 | 42.1 | 34.0 | 23.7 |
| 20.0 | 30.0 | 80.0 | 30.4 | 40.0 | 100.4 | 39.8 | 30.2 | 29.8 |
| 25.0 | 37.5 | 75.0 | 28.5 | 40.0 | 106 | 37.7 | 26.8 | 35.3 |
| | | | | | | Ca % | F % | Cytosine |
| Ca/F/Cytosine | | | F₂ | Ca | PM | w/w | w/w | % |
| % | 110 | | 38.0 | 40.0 | | | | |
| 0.0 | 0.0 | 100.0 | 38.0 | 40.0 | 78.0 | 51.3 | 48.7 | 0.0 |
| 2.0 | 2.2 | 98.0 | 37.2 | 40.0 | 79.4 | 50.3 | 46.8 | 2.7 |
| 5.0 | 5.5 | 95.0 | 36.1 | 40.0 | 81.6 | 49.0 | 44.2 | 6.7 |
| 10.0 | 11.0 | 90.0 | 34.2 | 40.0 | 85.2 | 46.9 | 40.1 | 12.9 |
| 15.0 | 16.5 | 85.0 | 32.3 | 40.0 | 88.8 | 45.0 | 36.3 | 18.5 |
| 20.0 | 22.0 | 80.0 | 30.4 | 40.0 | 92.4 | 43.2 | 32.9 | 23.8 |
| 25.0 | 27.5 | 75.0 | 28.5 | 40.0 | 96.0 | 41.6 | 29.6 | 28.6 |
| | | | | | | Ca % | F % | Thymine |
| Ca/F/Thymine | | | F₂ | Ca | PM | w/w | w/w | % |
| % | 125 | | 38.0 | 40.0 | 78.0 | 51.3 | 48.7 | 0.0 |
| 0.0 | 0.0 | 100.0 | 38.0 | 40.0 | 79.7 | 50.1 | 46.6 | 3.1 |
| 2.0 | 2.5 | 98.0 | 37.2 | 40.0 | 82.3 | 48.5 | 43.8 | 7.5 |
| 5.0 | 6.25 | 95.0 | 36.1 | 40.0 | 86.7 | 46.1 | 39.4 | 14.4 |
| 10.0 | 12.5 | 90.0 | 34.2 | 40.0 | 91.0 | 43.9 | 35.4 | 20.6 |
| 15.0 | 18.7 | 85.0 | 32.3 | 40.0 | 95.4 | 41.9 | 31.8 | 26.2 |
| 20.0 | 25.0 | 80.0 | 30.4 | 40.0 | 99.7 | 40.1 | 28.5 | 31.3 |
| 25.0 | 31.2 | 75.0 | 28.5 | 40.0 | 78.0 | 51.3 | 48.7 | 0.0 |

In some aspects, the calcium fluoride compositions disclosed herein are pharmaceutically acceptable.

In some aspects, the calcium fluoride composites disclosed herein are in particulate form. In some aspects, the composite particles are in the nanoparticles or microparticles size range.

By “nanoparticles” is intended particles in the range of 1 nm-999 nm, inclusive. Included within this definition are particles in the range of (A) between 50 nm and 100 nm, inclusive; between 45 nm and 110 nm, inclusive; between 40 nm and 120 nm, inclusive; between 35 nm and 130 nm, inclusive; between 30 nm and 140 nm, inclusive; between 25 nm and 150 nm, inclusive; between 20 nm and 160 nm, inclusive; between 15 nm and 170 nm, inclusive; between 10 nm and 180 nm, inclusive; (B) no less than 10 nm, no less than 15 nm, no less than 20 nm, no less than 25 nm; (C) no more than 150 nm, no more than 200 nm, no more than 250 nm, no more than 300 nm, no more than 350 nm, no more than 400 nm, no more than 450 nm, no more than 500 nm, no more than 550 nm, no more than 600 nm, no more than 650 nm, no more than 700 nm, no more than 750 nm, no more than 800 nm, no more than 850 nm; or (D) roughly around 25 nm.

By “microparticles” is intended particles within the range of 1 μm-999 μm, inclusive. Included within this definition are particles in the range of no more than 50 μm, no more than 100 μm, no more than 150 μm, no more than 200 μm, no more than 250 μm, no more than 300 μm, no more than 350 μm, no more than 400 μm, no more than 450 μm, no more than 500 μm, no more than 550 μm, no more than 600 μm, no more than 650 μm, no more than 700 μm, no more than 750 μm, no more than 800 μm, no more than 850 μm, no more than 900 μm, no more than 950 μm.

In some aspects, the calcium fluoride compositions disclosed herein comprise more than one composite, where each composite comprises Ca, F, and Z as disclosed in the preceding paragraphs, and where each composite differs from the other by the percentage w/w of Ca, F, or Z, or by the chemical structure of Z.

In some aspects, the calcium fluoride compositions disclosed herein comprise an antigen, where the antigen is adsorbed to a calcium fluoride composite.

By “antigen” is intended a protein, polysaccharide, peptide, nucleic acid, protein-polysaccharide conjugates, molecule or hapten that is capable of raising an immune response in a human or animal. Antigens may be derived, homologous or synthesized to mimic molecules from viruses, bacteria, parasites, protozoan or fungus. In an alternative embodiment of the invention the antigen derived, homologous or synthesized to mimic molecules from a tumor cell or neoplasia. In a further embodiment of the invention the antigen is derived, homologous or synthesized to mimic molecules from a substance implicated in allergy, Alzheimer's disease, atherosclerosis, obesity and nicotine-dependence.

Adsorption of albumin, chondroitin sulfate and glycoprotein onto calcium fluoride (Ca F₂) was described Lindemann (1985) Scandinavian Journal of Dental Research, 93:381-83. Adhesion of microorganisms on CaF₂ was reported Cheung et al. (2007) Journal of applied Microbiology 102:701-710). More recently, adsorption of ibuprofen on monodisperse CaF₂ hollow nano-spheres was described Zhang et al. (2010) Chem. Eur. J. 16:5672-5680. Adsorptions of antigen on inorganic material are carried out by mixing antigen, in appropriate buffer, to a water suspension of the inorganic material in nano- or microparticle form. Optimization of time, temperature, pH, presence of salts and excipients are selected according to the conditions known or determined for the antigen. Depending of the nature and chemical composition of the antigen, at least one of the following adsorption mode of interaction may occur: adsorption by ligand exchange, by electrostatic forces or by hydrophobic forces. Antigen/inorganic ratio are optimized on a case per case basis. Available inorganic surface can be increased by using particles of smaller sizes.

In some aspects, antigens are adsorbed at room temperature over 2 hours under gentle agitation. In some aspects, the process of making a calcium fluoride composition comprises a step of adsorbing one or more antigens to the calcium fluoride composite during formation of the calcium fluoride composite. In some aspects, the process of making a calcium fluoride composition comprises a step of adsorbing one or more antigens to the calcium fluoride composite after formation of the calcium fluoride composite.

For various organic compounds (Z), the charge measured at the surface of the particle varies (see table 2A). This particle property may be utilized to optimize antigen adsorption by the electrostatic mode of interaction.

In some aspects, the calcium fluoride compositions disclosed herein are used in stabilizing an antigen. In some aspects of this use, the antigen is thermostabilized. In some aspects of this use, the antigen is adsorbed to the calcium fluoride composite.

In some aspects, the calcium fluoride compositions disclosed herein are used in medicine. In some aspects, the calcium fluoride compositions disclosed herein are used in raising an immune response in a mammal. In some aspects, the calcium fluoride compositions disclosed herein are used in raising an immune response in a human. In some aspects, the calcium fluoride compositions disclosed herein are used in the prophylaxis and/or treatment of a mammal against disease caused by a virus, bacterium, or parasite. In some aspects, the calcium fluoride compositions disclosed herein are used in the prophylaxis and/or treatment of a human against disease caused by a virus, bacterium, or parasite. For such uses, the compositions disclosed herein may be delivered by administration to a subject in need thereof. Administration may be by a number of routes, including by delivery intramuscularly, subcutaneously, intradermally, sublingually, to the tonsils, or intranasally.

Processes for Making Compositions from Solid Particles

CaF₂ is available commercially. (Riedel de Haën™.) Pure CaF₂ for use in the compositions disclosed herein may be prepared from solid CaF₂ by the following scheme.

### Scheme 1:

1. CaF₂ solid particles are place in a container.
2. Water is added.
3. The CaF₂+water is mixed.
4. The mixture is allowed to stand.
5. Up to or more than ½ of the supernatant is removed and replaced by water.
6. Steps 3-5 are repeated.
7. CaF₂ solid is concentrated (by, e.g., centrifugation).

Composites for use in the compositions disclosed herein may be prepared by the following scheme. The starting constituents are available commercially.

### Scheme 2:

1. A solution comprising the selected compound Z is prepared.
2. CaF₂ solid particles are added.
3. The CaF₂ solid particles+solution comprising Z is mixed.
4. The mixture is allowed to stand.
5. Up to or more than ½ of the supernatant is removed and replaced by water.
8. Steps 3-5 are repeated.
6. The resulting solid is concentrated (by, e.g., centrifugation).
   Processes for Making Compositions from Aqueous Solutions

Methods for synthesizing CaF₂ from starting constituents in solution are known. For instance, preparation of nano-sized calcium fluoride by spray-drying following was reported by Sun et al. (2008) Dental materials 24:111-116, but this method has the disadvantage to use calcium hydroxide solution, which readily adsorbs CO₂ from the air giving unwanted calcium carbonate contamination. See Kalinkin (2005) Inorganic Materials 41:1073-1079. Nanoscale calcium fluoride may also be prepared according to Feldmann et al. (2006) Small 2:1248-1250, but this method has the disadvantage to use nitrate which even at trace level concentrations could be a problem for human injectable preparations. CaF₂ may be synthesized by sol gel precipitation methodology.

Sol gel precipitation methodology is described in Nandiyanto et al. (2011) “Liquid-phase Synthesis of CaF₂ Particles and Their Low Refractive Index Characterization” KONA Powder and Particle Journal 29:141-155. Nandiyanto indicates that certain parameters influence particle formation under the sol gel process. For instance, to influence the particle growing step, timing and temperature may be adjusted. Applicants modified the sol gel process by including washing steps as described generally in the following steps and in detail in the Examples. In some aspects, inclusion of washing steps is another way to decrease the particles growing step. It was observed that during washing by dilution, the concentration of starting materials was decreased. It is also expected that during washing, dilution of newly formed particles would occur. CaF₂ for use in the compositions disclosed herein may be prepared by sol gel precipitation according to Reaction I as modified by Scheme 3.

CaCl₂+NaF→CaF₍₂₎+NaCl Reaction I

### Scheme 3:

1. A solution comprising NaF is prepared (and sterilized by filtration). (NaF is available commercially.)
2. A solution comprising CaCl₂ is prepared (and sterilized by filtration). (CaCl₂ is available commercially.)
3. The solutions of step 1 and 2 are mixed.
4. The mixture is allowed to stand.
5. Up to or more than ½ of the supernatant is removed and replaced by water.
6. The retained liquid is mixed and the mixture is allowed to stand.
7. Steps 5-6 are repeated.
8. The resulting solid is concentrated (by, e.g., centrifugation).

Sol gel methodology was further modified for use in the present disclosure by the inclusion of Z in the reaction. In some aspects are provided processes for making a calcium fluoride composite by sol gel precipitation comprising the steps of combining CaCl₂, NaF, and NaZ under precipitating conditions and collecting the water insoluble calcium fluoride composite. In some aspects, the processes comprise a step of washing the calcium fluoride composite. In some aspects are provided processes for making a calcium fluoride composite by sol gel precipitation, comprising the steps of combining CaCl₂, NaF, and NaZ under precipitating conditions and collecting the water insoluble calcium fluoride composite.

Calcium fluoride composites for use in the compositions disclosed herein may be prepared according to Reaction II by following Scheme 4.

CaCl₂+NaF+AZ→CaF₍₂₋ₓ₎Zₓ/Z_{y}+NaCl Reaction II

where A is a metal, and x and y are as described in Formula I. In some aspects, A is Ca or Na.

### Scheme 4:

1. A solution comprising the selected NaZ is prepared (and sterilized by filtration).
2. A solution comprising NaF is prepared (and sterilized by filtration).
3. A solution comprising CaCl₂ is prepared (and sterilized by filtration).
4. The solutions of steps 1 and 2 are combined, then combined with the solution of step 3, then mixed.
5. The mixture is allowed to stand.
6. Up to or more than ½ of the supernatant is removed and replaced by water.
7. The retained liquid is mixed and the mixture is allowed to stand.
8. Steps 6-7 are repeated.
9. The resulting solid is concentrated (by, e.g., centrifugation).

Alternatively, calcium fluoride composites for use in the compositions disclosed herein may be prepared according to Reaction II by following Scheme 5.

### Scheme 5:

1. A solution comprising NaF is prepared (and sterilized by filtration).
2. A solution comprising CaCl₂ and the selected organic Z is prepared (and sterilized by filtration).
3. The solutions of steps 1 and 2 are mixed.
4. The mixture is allowed to stand.
5. Up to or more than ½ of the supernatant is removed and replaced by water.
6. The retained liquid is mixed and the mixture is allowed to stand.
7. Steps 5-6 are repeated.
8. The resulting solid is concentrated (by, e.g., centrifugation).

Alternatively, calcium fluoride composites for use in the compositions disclosed herein may be prepared according to Reaction II by following Scheme 6.

### Scheme 6:

1. A solution comprising NaF and the selected organic Z is prepared (and sterilized by filtration).
2. A solution comprising CaCl₂ is prepared (and sterilized by filtration).
3. The solutions of steps 1 and 2 are mixed.
4. The mixture is allowed to stand.
5. Up to or more than ½ of the supernatant is removed and replaced by water.
6. The retained liquid is mixed and the mixture is allowed to stand.
7. Steps 5-6 are repeated.
8. The resulting solid is concentrated (by, e.g., centrifugation).

Alternatively, calcium fluoride composites for use in the compositions disclosed herein may be prepared using calcium ascorbate according Scheme 7.

### Scheme 7:

1. A solution comprising CaC₁₂H₁₄O₁₂ is prepared (and sterilized by filtration).
2. A solution comprising NaF is prepared (and sterilized by filtration).
3. The solutions of steps 1 and 2 are mixed.
4. The mixture is allowed to stand.
5. Up to or more than ½ of the supernatant is removed and replaced by water.
6. The retained liquid is mixed and the mixture is allowed to stand.
7. Steps 5-6 are repeated.
8. The resulting solid is concentrated (by, e.g., centrifugation).

In some aspects, the process of making a calcium fluoride composition comprises combining one or more antigens with CaCl2, NaF, and NaZ under precipitating conditions. In some aspects, the process of making a calcium fluoride composition comprises a step of washing the calcium fluoride composite, wherein the washing step further comprises combining one or more antigens with the calcium fluoride composite. In some aspects, the process of making a calcium fluoride composition comprises a step of mixing the calcium fluoride composite with one or more antigens.

In some aspects, products made by the processes describe herein are disclosed.

### Adjuvant Compositions

In some aspects are provided an adjuvant composition comprising a calcium fluoride composition as disclosed herein. By “adjuvant composition” is intended a calcium fluoride composition as disclosed herein that is capable of increasing an immune response against an antigen compared to administration of said antigen alone. In some aspects, adjuvant compositions as disclosed herein further comprise an immunostimulant.

In one aspect, this immunostimulant may be a saponin. A particularly suitable saponin for use in the present invention is Quil A and its derivatives. Quil A is a saponin preparation isolated from the South American tree Quillaja Saponaria Molina and was first described by Dalsgaard et al. in 1974 (“Saponin adjuvants”, Archiv. fur die gesamte Virusforschung, Vol. 44, Springer Verlag, Berlin, p243-254) to have adjuvant activity. Purified fragments of Quil A have been isolated by HPLC which retain adjuvant activity without the toxicity associated with Quil A (EP 0 362 278), for example QS7 and QS21 (also known as QA7 and QA21). QS-21 is a natural saponin derived from the bark of Quillaja saponaria Molina, which induces CD8+ cytotoxic T cells (CTLs), Th1 cells and a predominant IgG2a antibody response. QS21 is a preferred saponin in the context of the present invention.

In a suitable form of the present invention, the saponin adjuvant within the adjuvant composition is a derivative of saponaria molina quil A, preferably an immunologically active fraction of Quil A, such as QS-17 or QS-21, suitably QS-21.

In a specific aspect, QS21 is provided in its less reactogenic composition where it is quenched with an exogenous sterol, such as cholesterol for example. Several particular forms of less reactogenic compositions wherein QS21 is quenched with an exogenous cholesterol exist. In a specific aspect, the saponin/sterol is in the form of a liposome structure (WO 96/33739). In this aspect the liposomes suitably contain a neutral lipid, for example phosphatidylcholine, which is suitably non-crystalline at room temperature, for example eggyolk phosphatidylcholine, dioleoyl phosphatidylcholine (DOPC) or dilauryl phosphatidylcholine. The liposomes may also contain a charged lipid which increases the stability of the lipsome-QS21 structure for liposomes composed of saturated lipids. In these cases the amount of charged lipid is suitably 1-20% w/w, preferably 5-10%. The ratio of sterol to phospholipid is 1-50% (mol/mol), suitably 20-25%.

Suitable sterols include 6-sitosterol, stigmasterol, ergosterol, ergocalciferol and cholesterol. In one particular aspect, the adjuvant composition comprises cholesterol as sterol. These sterols are well known in the art, for example cholesterol is disclosed in the Merck Index, 11th Edn., page 341, as a naturally occurring sterol found in animal fat.

Where the active saponin fraction is QS21, the ratio of QS21: sterol will typically be in the order of 1:100 to 1:1 (w/w), suitably between 1:10 to 1:1 (w/w), and preferably 1:5 to 1:1 (w/w). Suitably excess sterol is present, the ratio of QS21:sterol being at least 1:2 (w/w). In one aspect, the ratio of QS21:sterol is 1:5 (w/w). The sterol is suitably cholesterol.

In another aspect, the adjuvant composition comprises an immunostimulant which is a Toll-like receptor 4 (TLR4) agonist. By “TLR agonist” it is meant a component which is capable of causing a signaling response through a TLR signaling pathway, either as a direct ligand or indirectly through generation of endogenous or exogenous ligand (Sabroe et al, JI 2003 p1630-5). A TLR4 agonist is capable of causing a signally response through a TLR-4 signaling pathway. A suitable example of a TLR4 agonist is a lipopolysaccharide, suitably a non-toxic derivative of lipid A, particularly monophosphoryl lipid A or more particularly 3-Deacylated monophoshoryl lipid A (3D-MPL).

3D-MPL is sold under the name MPL by GlaxoSmithKline Biologicals N.A. and is referred throughout the document as MPL or 3D-MPL. see, for example, U.S. Pat. Nos. 4,436,727; 4,877,611; 4,866,034 and 4,912,094. 3D-MPL primarily promotes CD4+T cell responses with an IFN-g (Th1) phenotype. 3D-MPL can be produced according to the methods disclosed in GB 2 220 211 A. Chemically it is a mixture of 3-deacylated monophosphoryl lipid A with 3, 4, 5 or 6 acylated chains. In the compositions of the present invention small particle 3D-MPL may be used to prepare the adjuvant composition. Small particle 3D-MPL has a particle size such that it may be sterile-filtered through a 0.22 μm filter. Such preparations are described in WO 94/21292. Preferably, powdered 3D-MPL is used to prepare the adjuvant compositions of the present invention.

Other TLR4 agonists which can be used are aminoalkyl glucosaminide phosphates (AGPs) such as those disclosed in WO98/50399 or U.S. Pat. No. 6,303,347 (processes for preparation of AGPs are also disclosed), suitably RC527 or RC529 or pharmaceutically acceptable salts of AGPs as disclosed in U.S. Pat. No. 6,764,840. Some AGPs are TLR4 agonists, and some are TLR4 antagonists. Both are thought to be useful as immunostimulants.

Other suitable TLR-4 agonists are as described in WO2003/011223 and in WO 2003/099195, such as compound I, compound II and compound III disclosed on pages 4-5 of WO2003/011223 or on pages 3-4 of WO2003/099195 and in particular those compounds disclosed in WO2003/011223 as ER803022, ER803058, ER803732, ER804053, ER804057m ER804058, ER804059, ER804442, ER804680 and ER804764. For example, one suitable TLR-4 agonist is ER804057.

In a particular aspect, the adjuvant composition comprises both saponin and a TLR4 agonist. In a specific example, the adjuvant composition comprises QS21 and 3D-MPL.

A TLR-4 agonist such as a lipopolysaccharide, such as 3D-MPL, can be used at amounts between 1 and 100 μg per human dose of the adjuvant composition. 3D-MPL may be used at a level of about 50 μg, for example between 40 to 60 μg, suitably between 45 to 55 μg or between 49 and 51 μg or 50 μg. In a further aspect, the human dose of the adjuvant composition comprises 3D-MPL at a level of about 25 μg, for example between 20 to 30 μg, suitable between 21 to 29 μg or between 22 to 28 μg or between 28 and 27 μg or between 24 and 26 μg, or 25 μg.

A saponin, such as QS21, can be used at amounts between 1 and 100 μg per human dose of the adjuvant composition. QS21 may be used at a level of about 50 μg, for example between 40-60 μg, suitably between 45 to 55 μg or between 49 and 51 μg or 50 μg. In a further aspect, the human dose of the adjuvant composition comprises QS21 at a level of about 25 μg, for example between 20 to 30 μg, suitable between 21 to 29 μg or between 22 to 28 μg or between 28 and 27 μg or between 24 and 26 μg, or 25 μg.

Where both TLR4 agonist and saponin are present in the adjuvant composition, then the weight ratio of TLR4 agonist to saponin is suitably between 1:5 to 5:1, suitably 1:1. For example, where 3D-MPL is present at an amount of 50 μg or 25 μg, then suitably QS21 may also be present at an amount of 50 μg or 25 μg, respectively, per human dose of the adjuvant composition.

In one aspect, the immunostimulant is a TLR9 agonist, for example as set out in WO 2008/142133. In a specific example, said TLR9 agonist is an immunostimulatory oligonucleotide, in particular an oligonucleotide containing an unmethylated CpG motif. Such oligonucleotides are well known and are described, for example, in WO 96/02555, WO 99/33488 and U.S. Pat. No. 5,865,462. Suitable TLR9 agonists for use in the adjuvant compositions described herein are CpG containing oligonucleotides, optionally containing two or more dinucleotide CpG motifs separated by at least three, suitably at least six or more nucleotides. A CpG motif is a cytosine nucleotide followed by a Guanine nucleotide.

In one aspect the internucleotide bond in the oligonucleotide is phosphorodithioate, or possibly a phosphorothioate bond, although phosphodiester and other internucleotide bonds could also be used, including oligonucleotides with mixed internucleotide linkages. Methods for producing phosphorothioate oligonucleotides or phosphorodithioate are described in U.S. Pat. No. 5,666,153, U.S. Pat. No. 5,278,302 and WO95/26204. Oligonucleotide comprising different internucleotide linkages are contemplated, e.g. mixed phosphorothioate phophodiesters. Other internucleotide bonds which stabilize the oligonucleotide may be used.

Examples of CpG oligonucleotides suitable for inclusion in the adjuvant compositions described herein have the following sequences. In one aspect, these sequences contain phosphorothioate modified internucleotide linkages.

CHART 2
CpG oligos.

| CpG | | SEQ |
|---|---|---|
| No. | Sequence | ID NO |
| 1826 | TCC ATG ACG TTC CTG ACG TT | 1 |
| 1758 | TCT CCC AGC GTG CGC CAT | 2 |
| 1212 | ACC GAT GAC GTC GCC GGT GAC GGC | 3 |
| | ACC ACG | |
| 2006/ | TCG TCG TTT TGT CGT TTT GTC GTT | 4 |
| 7909 | | |
| 1668 | TCC ATG ACG TTC CTG ATG CT | 5 |
| 5456 | TCG ACG TTT TCG GCG CGC GCC G | 6 |

Alternative CpG oligonucleotides may comprise the sequences above in that they have inconsequential deletions or additions thereto.

In one aspect the immunostimulant is a tocol. Tocols are well known in the art and are described in EP0382271. In a particular aspect, the tocol is alpha-tocopherol or a derivative thereof such as alpha-tocopherol succinate (also known as vitamin E succinate).

In one aspect, adjuvant compositions disclosed herein comprise an immunostimulant adsorbed to a calcium fluoride composite. In one aspect, adjuvant compositions comprise an immunostimulant adsorbed to a calcium fluoride composite, wherein said immunostimulant adsorbed to a calcium fluoride composite is MPL.

In one aspect is disclosed the adjuvant compositions disclosed herein for use in increasing the immune response against an antigen compared to an immune raised against said antigen when said antigen is administered with calcium fluoride composition or alone. In one aspect is disclosed the adjuvant compositions disclosed herein for use in increasing the immune response against an antigen compared to an immune raised against said antigen when said antigen is administered with calcium phosphate. The compositions disclosed herein may be delivered by administration to a subject in need thereof. Administration may be by a number of routes, including by delivery intramuscularly, subcutaneously, intradermally, sublingually, to the tonsils, or intranasally.

### Processes for Making Adjuvant Compositions

In some aspects are disclosed processes for making an adjuvant composition as disclosed herein, comprising the steps of combining an immunostimulant with a calcium fluoride composite described herein. In some aspects are disclosed processes for making an adjuvant composition as disclosed herein, comprising the steps of adsorbing an antigen to a calcium fluoride composite as described herein.

### Immunogenic Compositions

In some aspects are provided an immunogenic composition comprising an antigen and an adjuvant composition as described herein. In some aspects are provided an immunogenic composition as disclosed herein to be delivered intramuscularly, subcutaneously, intradermally, sublingually, to the tonsils, or intranasally.

In some aspects are provided an immunogenic composition as disclosed herein, where the composition wherein the pH of said composition is between about pH5 and pH9. In some aspects are provided immunogenic compositions as disclosed herein that is suitable for human administration. In some aspects are provided an immunogenic composition as disclosed herein comprising one or more pharmaceutically acceptable excipients, in particular a buffer, a Tris buffer; or a histidine buffer. In some aspects are provided an immunogenic composition as disclosed herein, wherein the composition is prepared under asceptic conditions. In some aspects are provided an immunogenic composition as disclosed herein, wherein the composition is non-pyrogenic. In some aspects are provided an immunogenic composition as disclosed herein, where the composition is isotonic. In some aspects are provided an immunogenic composition as disclosed herein, where the composition comprises sugar or polyols.

In some aspects are provided an immunogenic composition as disclosed herein, where at least one antigen and at least one immunostimulant are adsorbed to a single type of composite as defined by percent w/w Ca, F, and Z and chemical structure of Z. In some aspects are provided an immunogenic composition as disclosed herein, where more than one antigen and more than one immunostimulant are adsorbed to a single type of composite as defined by percent w/w Ca, F, and Z and chemical structure of Z. In some aspects are provided an immunogenic composition as disclosed herein comprising at least a first and second type of composite as defined by percent w/w Ca, F, and Z and chemical structure of Z, wherein at least one antigen, at least one immunostimulant, or both, is adsorbed to said first type of composite, and wherein at least one antigen, at least one immunostimulant, or both, is adsorbed to said second type of composite. In some aspects are provided an immunogenic composition as disclosed herein comprising at least one composite as defined by percent w/w Ca, F, and Z and chemical structure of Z, wherein at least one antigen, at least one immunostimulant, or both, is adsorbed to said at least one composite, and wherein at least one antigen, at least one immunostimulant, or both, is adsorbed to a different metallic salt adjuvant. In some aspects, the second metallic salt adjuvant is calcium phosphate.

In some aspects there is provided immunogenic compositions disclosed herein for use in increasing the immune response against an antigen compared to an immune raised against said antigen when said antigen is administered with calcium fluoride composition or alone. In one aspect there is provided immunogenic compositions disclosed herein for use in increasing the immune response against an antigen compared to an immune raised against said antigen when said antigen is administered with calcium phosphate. The compositions disclosed herein may be delivered by administration to a subject in need thereof. Administration may be by a number of routes, including by delivery intramuscularly, subcutaneously, intradermally, sublingually, to the tonsils, or intranasally.

### Processes for Making Immunogenic Compositions

In some aspects are provided processes for making an immunogenic composition as disclosed herein, comprising the steps of combining a calcium fluoride composition described herein with an adjuvant composition disclosed herein.

### Compositions and Methods for Reducing QS21 Reactogenicity

As described previously herein, QS21 is reactogenic and is typically provided in its less reactogenic composition (where it is quenched with an exogenous sterol, such as cholesterol for example). Several particular forms of less reactogenic compositions wherein QS21 is quenched with an exogenous cholesterol exist.

It was observed herein that QS21 combined with a calcium fluoride composition wherein Z is glutathione exhibits hemolytic activity levels (as measured by in vitro assay) comparable to the less compositions wherein QS21 is quenched with an exogenous cholesterol.

Accordingly, in some aspects are provided an adjuvant composition comprising a calcium fluoride composition as defined herein, wherein Z is glutathione, further comprising an immunologically active saponin fraction. In some aspects are provided an adjuvant composition, wherein the immunologically active saponin fraction is QS21. In some aspects are provided an adjuvant composition, wherein the QS21 is adsorbed to a calcium fluoride composite wherein Z is glutathione. In some aspects are provided an adjuvant composition wherein Z is glutathione for use in decreasing the hemolytic activity of QS21. In some aspects are provided a method for reducing the hemolytic activity of QS21 comprising the steps of combining QS21 with a calcium fluoride composite, wherein Z is glutathione. In some aspects are provided an immunogenic composition comprising an antigen and an adjuvant composition defined in the preceding paragraph. A suitable test to determine haemolytic activity of immunologically active saponin fractions, including QS21, is described in Rönnberg et al. (1995) Vaccine 13:1375-1382.

### Methods for Using Compositions

Methods are provided for the treatment or prevention of an infection or a disease caused by a virus, bacterium, or parasite in a mammal, said method comprising administering to said mammal a therapeutically effective amount of the calcium fluoride composition, the adjuvant composition, or the immunogenic composition described herein. Methods are provided for the treatment or prevention of an infection or a disease caused by a virus, bacterium, or parasite in a human, said method comprising administering to said human a therapeutically effective amount of the calcium fluoride composition, the adjuvant composition, or the immunogenic composition described herein.

Methods are provided for inducing an immunogenic response in a mammal in need thereof, said method comprising administering to said mammal an effective amount of the calcium fluoride composition, the adjuvant composition, or the immunogenic composition described herein. Methods are provided for inducing an immunogenic response in a human in need thereof, said method comprising administering to said human an effective amount of the calcium fluoride composition, the adjuvant composition, or the immunogenic composition described herein.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. The term “plurality” refers to two or more. Additionally, numerical limitations given with respect to concentrations or levels of a substance, such as solution component concentrations or ratios thereof, and reaction conditions such as temperatures, pressures and cycle times are intended to be approximate. The term “about” used herein is intended to mean the amount ±10%.

The invention will be further described by reference to the following, non-limiting, figures and examples.

### EXAMPLES

### Analytical Methods

### Equal Compensation Point

Equal Compensation Point measurements: Measurements of Equal Compensation Point (E.C.P.) were carried out by potentiometric titration (J. R. Feldkamp et al., Journal of Pharmaceutical Sciences, 1981, Vol. 70, n° 6 p 638-640). The results were presented in a global graph which is obtained by the juxtaposition of 4 different titration curves: two of them being measured in water and the two others measured in presence of various KCl (or KNO3) concentrations. For example in batch Ca/F/CO3 #8833172A two Equal Compensation Point (E.C.P.) were obtained: 6.4 & 8.7 in the H2O/KCl system. In this case, below pH 6.4, the particle surface is charged negatively, between pH 6.4 and pH 8.7 the particle surface is charged positively, and above pH 8.7 the surface particle is charge negatively (Scheme 8). For comparison between the obtained E.C.P. H₂O/KNO3 values see Table 2A (herein below).

### Dry Weight

After homogenization of the suspension, an aliquot (10 ml) is evaporated to dry at 80° C. during 5 days. The weight of the sample (in mg), represents the dry material quantity present in 10 ml of the suspension. This weight divided by ten represents the dry material quantity/ml of suspension.

### Infrared Spectra

The dry material (obtained as described herein) is hand ground and used as such for the infrared analysis. Few mg of sample were placed on the multi reflection holder of the Perkin Elmer FT-Infra Red instrument. Spectra were scanned in the % of transmittance mode from 4000 cm-1 to 600 cm-1. It is interesting to note that organic material adsorbed on inorganic material give always very broad signals in infrared spectroscopy (compared to the pure organic material which gives very sharp signals).

Examples of this were described for ibuprofen adsorbed on CaF2 hollow sphere (C. Zhang et al., 2010, Chem. Eur. J. vol 16 p.5672-5680) or oleatate adsorbed fluorite (CaF2) (Handbook of Infrared Spectroscopy of Ultrathin Films. V. T. Tolstoy, I. V. Chernyshova and V. A. Skryshevsky. 2003 John Wiley & Sons, Inc. (page 552).

### Nitrogen Content by Antek

Suspensions were injected without any other treatment in the Antex instrument. Therefore N concentrations were expressed in μgN/ml and represent the total N content found for both supernatant solution and adsorbed material. Analyses were also made on the last washing supernatant (W-10).

### Ca and F Elementary Analyses

Suspensions containing 500 mg dry material were filtered to recover the solid parts, which were calcinated. After mineralization, one part is used for Ca % determination (+/−0.5%) and the other part for F % determination (+/−1%).

### Anti-Oxidant Capacity

Potassium permanganate (KMnO4), in presence of sulfuric acid solution, is one of the strongest oxidant. Violet permanganate anion is reduced to manganate oxide (MnO2 brawn color). This can further be reduced according to incolor Mn++ cation, resulting in a 5 electrons exchange. In such conditions, most of organic matters were fully oxidized, while inorganic matters, such as CaF2, were insensitive.

MnO₄− (violet color)+4H+ +3e− →MnO₂+2H₂O

MnO₂+4H+ +2e−→Mn++ (incolor)+2H₂O

Typically, five samples (0.1; 0.2; 0.3; 0.4 and 0.5 ml of suspension) were placed in transparent polymeric container (avoid glass container when fluoride derivatives were placed in acid medium). To each of them 1 ml of H₂SO₄ 5M is added (add acid to water and never the reverse). Titration is carried out by drop by drop addition of KMnO4 1.0 mM (violet color) until the discoloration persists during 3 minutes. Thus, anti-oxidant capacity, expressed in μl of KMnO4 1.0 mM/ml of suspension, is obtained. Those values can be converted in μl of KMnO₄ 1.0 mM/mg dry material. Similar titrations were carried out with known quantities of cysteine or N-acetyl-cysteine solutions (1.0 mM). Thus, correlations between consumed quantities of KMnO₄ and cysteine or N-acetyl-cysteine contents can be established. Furthermore, taking in account the weight of dry material present in the suspension, the quantities of organic materials (cysteine or N-acetyl-cysteine) per dry material can be calculated and expressed in % w/w oxidizable organic material/dry weight.

### Commercially Available Chemicals

The following commercial products have been used:
CaCO₃ solid: OMYA™ product OMYAPURE 35;
CaCO₃ solid particles:Sigma-Aldrich product 12010;
Sodium fluoride: Merck™ product 1064490250;
Calcium chloride: Merck™ product n° 1023780500;
Cysteine: Aldrich™ product 168149; Cysteine: Merck product 1028380100;
N-Acetyl-Cysteine: Sigma™ product A5099;
Thioglycerol: Sigma™ product 88640;
Phosphoethanolamine: Sigma™ product P0503-100;
Calcium fluoride: Riedel de Haën™ product 01123;
Sodium bicarbonate:Merck™ product 1063295000;
Sodium carbonate: Merck™ product 1063981000;
Calcium chloride dihydrate (CaCl₂.2H₂O): Merck™ product 2382;
Calcium chloride dihydrate (CaCl₂.2H₂O): Sigma Aldrich™ product 12022;
Disodium hydrogenophosphate dihydrate: Merck™ product 1065805000;
Tri-sodium citrate: Merck™ product 1110371000;
Sodium hydroxide: Merck™ product 1064981000;
Calcium ascorbate: Fluke™ product 11138;
Glutathione: Merck™ product 104090.0050;
Glutathione oxide: Sigma™ product G46265G;
Thiolactic acid:Sigma Aldrich™ product T3, 100-3;
Adipic acid: Carlo ERBA™ product 401785;
Uric acid: Fluke™ product 51449;
Calcium chloride hexahydrate: Merck™ product 102072.1000;
Folic acid: Fluke™ product 01769;
Hypoxanthine: Fluke product 56700
Xanthine: Sigma™ product X7375-256
Guanine: Aldrich™ product G11950-100G
Cytosine: Fluka™ product 30430
Thymine: Sigma™ product T0376-5G.

### Example 1

### Characteristics of Sol-Gel Formations of Calcium Fluoride

Calcium Fluoride composites were formed and characterized by various methods. The results of this characterization are summarized in this example. The details of the formation of each batch mentioned in Example 1 may be found in Example 2.

TABLE 1
Example of Calcium fluoride composites

| 1 | 2 | 3 | 4 | 5 | 6 | 7 | 8 | 9 |
|---|---|---|---|---|---|---|---|---|
| | | | | | | | | Total |
| | | | | | Started | Final | Dry | dry |
| | | NaF | CaCl₂ | Org. | volume | volume | conc. | weight |
| | Batch # | g | | | ml | ml | mg/ml | g |
| CaF₂ | | | | | | | | |
| CaF₂ | 8833172D | 1.68 | 2.22 | | 200 | 35 | 25.2 | 0.882 |
| CaF₂ | 8833190 | 8.42 | 11.11 | | 1000 | 160 | 33.21 | 5.313 |
| CaF₂ | 9440194 | 8.42 | 11.1 | | 1000 | 160 | 26.30 | 4.208 |
| Ca/F/OH | | | | | | | | |
| CaF/OH | 11000123 | 4.2 | 11.11 | | 1000 | 110 | 14.25 | 1.567 |
| Ca/F/CO₃ | | | | | | | | |
| Ca/F/CO₃ | 8833152 | 0.168 | 2.22 | 1.908 | 200 | 35 | 45.2 | 1.582 |
| Ca/F/CO₃ | 8833153 | 0.504 | 2.22 | 1.484 | 200 | 35 | 41.7 | 1.459 |
| Ca/F/CO₃ | 8833154 | 0.841 | 2.22 | 1.060 | 200 | 35 | 40.5 | 1.417 |
| Ca/F/CO₃ | 8833155 | 1.178 | 2.22 | 0.636 | 200 | 35 | 42.7 | 1.494 |
| Ca/F/CO₃ | 8833156 | 1.514 | 2.22 | 0.212 | 200 | 35 | 39.2 | 1.372 |
| Ca/F/CO₃ | 8833157 | 1.598 | 2.22 | 0.106 | 200 | 35 | 16.0 | 0.560 |
| Ca/F/CO₃ | 8833172A | 1.51 | 2.22 | 0.168 | 200 | 35 | 8.28 | 0.289 |
| Ca/F/CO₃ | 8833172B | 1.59 | 2.22 | 0.084 | 200 | 35 | 9.37 | 0.328 |
| Ca/F/CO₃ | 8833172C | 1.66 | 2.22 | 0.016 | 200 | 35 | 18.5 | 0.647 |
| Ca/F/CO₃ | 9440195 | 7.57 | 11.10 | 0.84 | 1000 | 160 | 14.6 | 2.336 |
| Ca/F/CO₃ | 9923123 | 7.58 | 11.13 | 0.84 | 1000 | 160 | 16.27 | 2.603 |
| Ca/F/CO₃ | 9923124 | 7.58 | 11.11 | 0.84 | 1000 | 160 | 19.67 | 3.147 |
| Ca/F/CO₃ | 11000080 | 0.84 | 11.1 | 7.70 | 1000 | 120 | 110.28 | 13.233 |
| Ca/F/CO₃ | 11000081 | 4.2 | 11.1 | 4.28 | 1000 | 170 | 40.79 | 6.934 |
| Ca/F/CO₃ | 11000082 | 5.88 | 11.1 | 2.57 | 1000 | 200 | 33.73 | 6.746 |
| Ca/F/CO₃ | 11000083 | 7.56 | 11.1 | 0.86 | 1000 | 180 | 23.56 | 4.241 |
| Ca/F/Ascorbate | | | | | | | | |
| Ca/F/Ascorbate | 9440198 | 4.20 | Ca ascor. | | 1000 | 250 | 13.5 | 3.375 |
| | | | 42.5 | | | | | |
| Ca/F/Cysteine | | | | | | | | |
| Ca/F/Cysteine* | 9440055 | 0.84 | 2.22 | 2.42 | 200 | 35 | 10.81 | 0.378 |
| Ca/F/Cysteine* | 9440056 | 0.84 | 2.22 | 2.42 | 200 | 35 | 13.20 | 0.462 |
| Ca/F/Cysteine* | 9440057 | 0.84 | 2.22 | 2.42 | 200 | 35 | 17.08 | 0.597 |
| Ca/F/Cysteine* | 9440058 | 0.84 | 2.22 | 2.42 | 200 | 35 | 8.72 | 0.305 |
| Ca/F/Cysteine* | 9440099 | 4.23 | 11.15 | 12.11 | 1000 | 160 | 20.84 | 3.334 |
| Ca/F/Cysteine* | 9440197 | 4.21 | 11.10 | 12.10 | 1000 | 160 | 20.00 | 3.200 |
| Ca/F/N-Ac-Cyst. | | | | | | | | |
| Ca/F/N-Ac-Cyst. | 9440110 | 4.20 | 11.10 | 16.37 | 1000 | 160 | 10.36 | 1.657 |
| Ca/F/N-Ac-Cyst. | 9440196 | 4.20 | 11.11 | 16.30 | 1000 | 160 | 9.34 | 1.494 |
| Ca/F/N-Ac-Cyst. | 10616125 | 4.20 | 11.13 | 16.33 | 1000 | 160 | 9.30 | 1.488 |
| Ca/F/N-Ac-Cyst. | 11000101 | 4.21 | 11.17 | 16.34 | 1000 | 150 | 14.9 | 2.235 |
| Ca/F/Glutathione | | | | | | | | |
| CaF/Glutathione | 10616185 | 4.21 | 11.10 | 30.7 | 1000 | 250 | 18.43 | 4.607 |
| CaF/Glutathione | 11000030 | 4.22 | 11.12 | 3.07 | 1000 | 160 | 27.2 | 4.352 |
| CaF/Glutathione | 11000033 | 4.21 | 11.12 | 30.7 | 1000 | 175 | 23.5 | 4.112 |
| CaF/Glutathione | 11000086 | 4.22 | 11.15 | 3.07 | 1000 | 170 | 24.76 | 4.209 |
| CaF/Glutathione | 11000099 | 4.11 | 11.12 | 3.07 | 1000 | 180 | 23.19 | 4.174 |
| CaF/Glutathione | 11000194 | 4.20 | 11.13 | 3.09 | 1000 | 200 | 22.02 | 4.404 |
| Ca/F/Glutathione oxide | | | | | | | | |
| CaF/Glutathi. Oxi | 10616198 | 0.4 | 1.11 | 6.56 | 100 | 50 | 11.0 | 0.550 |
| CaF/Glutathi. Oxi | 11000139 | 4.20 | 11.15 | 3.22 | 1000 | 220 | 22.12 | 4.866 |
| CaF/Glutathi. Oxi | 11000140 | 4.20 | 11.11 | 6.56 | 1000 | 220 | 22.6 | 4.972 |
| Ca/F/thiolactate | | | | | | | | |
| Ca/F/Thiolactate | 11000031 | 4.21 | 11.12 | 10.6 | 1000 | 170 | 23.8 | 4.046 |
| Ca/F/Thiolactate | 11000059 | 4.21 | 11.17 | 10.6 | 1000 | 160 | 10.6 | 1.696 |
| Ca/F/Thiolactate | 11000060 | 4.12 | 11.17 | 10.6 | 1000 | 200 | 19.3 | 3.860 |
| Ca/F/Adipate | | | | | | | | |
| Ca/F/Adipate | 11000129 | 4.19 | 11.14 | 7.3 | 1000 | 150 | 29.9 | 4.485 |
| Ca/F/Adipate | 11481026 | 4.19 | 11.12 | 7.31 | 1000 | 174 | 26.32 | 4.579 |
| Ca/F/Adipate | 11481027 | 4.19 | 11.11 | 7.31 | 1000 | 142 | 31.61 | 4.488 |
| Ca/F/Urate | | | | | | | | |
| Ca/F/Urate | 11000182 | 4.16 | 21.99 | 0.132 | 1000 | 150 | 12.89 | 1.933 |
| | | | •6H2O | | | | | |
| Ca/F/Folic acid | | | | | | | | |
| Ca/F/Folic acid | 11481018 | 4.20 | 11.12 | 0.448 | 1000 | 250 | 15.01 | 3.752 |
| Ca/F/Hypoxanthine | | | | | | | | |
| Ca/F/Hypoxanthine | 11481198 | 4.21 | 14.73 | 1.36 | 1000 | 200 | 13.09 | 2.618 |
| Ca/F/Xanthine | | | | | | | | |
| Ca/F/Xanthine | 11481199 | 4.22 | 14.71 | 1.52 | 1000 | 200 | 20.3 | 4.060 |
| Ca/F/Guanine | | | | | | | | |
| Ca/F/Guanine | 11481195 | 4.21 | 14.71 | 1.52 | 1000 | 180 | 21.58 | 3.884 |
| Ca/F/Cytosine | | | | | | | | |
| Ca/F/Cytosine | 11954009 | 4.22 | 14.73 | 1.10 | 1000 | 100 | 13.74 | 1.374 |
| Ca/F/Thymine | | | | | | | | |
| Ca/F/Thymine | 11954064 | 4.22 | 14.75 | 1.26 | 1000 | 180 | 9.8 | 1.767 |

Quantities of NaF, CaCl₂, and organic materials: column 3, 4, and 5 respectively.
Volume of the starting mix = Column 6.
Final volume after concentration by centrifugation: column 7.
Concentration of dry material: column 8.
Total weight (column 9) is defined as the product of the concentration (mg dry material/ml column 8) by total volume at the final stage of preparation (ml in column 7).

Sol-gel formation allows one to influence the particle size by, for example, varying concentrations of starting solutions as disclosed in Nandiyanto. The use of various selected organic compounds in solutions allows one to obtain composite particles possessing different surface charges (measured by their E.C.P. values, Table 2A).

TABLE 2A
Starting solutions pH and surface charge (E.C.P.)
of calcium fluoride composites.

| | | Starting pH of | |
|---|---|---|---|
| Starting | Batch | each solutions | E.C.P. |
| compounds | # | and →pH of first wash | H₂O/KNO₃ |
| CaF₂ | 8833190 | 7.15 + 7.11 → 9.44 | 6.9 |
| | 9440194 | 9.92 + 10.04→9.78 | 6.4 |
| Ca/F/N-Ac- | 9440110 | 8.35 + 9.26 → 8.47 | 7.3 |
| Cysteine | 9440196 | 9.66 + 8.35 → 8.44 | 7.3 |
| | 10616125 | 9.01 + 8.30 → 8.44 | 7.3 |
| Ca/F/Ascorbate | 9440198 | 9.08 + 9.49 → 6.65 | 7.3 |
| Ca/F/CO₃ | 9440195 | 7.29 + 7.96→ 6.38 | 8.6 |
| | 9923123 | 7.25 + 7.72→ 6.43 | 8.3 |
| | 9923124 | 7.27 + 7.70→ 6.46 | 8.3 |
| Ca/F/Cysteine | 9440099 | 8.22 + 9.22 → 8.27 | 8.4 |
| | 9440197 | 9.62 + 8.21 → 8.31 | 8.4 |
| Ca/F/Uric acid | 11000182 | 8.60 + 6.24→ 5.39 | 8.4 |
| Ca/F/Glutathione | 11000139 | 9.82 + 8.21 → 7.84 | 9.3 |
| oxide | 11000140 | 9.82 + 8.12 → 8.07 | 9.8 |
| Ca/F/ | 11000033 | 6.94 + 7.04 → 7.07 | 8.8 |
| Glutathione | 10616185 | 8.90 + 8.57 → 8.74 | 9.1 |
| | 11000030 | 9.47 + 8.59 → 8.51 | 9.2 |
| Ca/F/ | 11000059 | 7.16 + 6.97 → 6.17 | 6.1 |
| Thiolactate | 11000031 | 9.56 + 9.54 → 9.73 | 6.7 |
| | 11000060 | 7.93 + 8.07 → 7.93 | 7.1 |
| | 11481063 | 9.15 + 8.50 → 8.87 | 6.9 |
| | 11481062 | 8.93 + 9.49 → 10.14 | 7.8 |
| Ca/ | 11000129 | 8.02 + 10.81→ 7.97 | 6.6 |
| Adipate | 11481044 | 6.99 + 9.51→ 6.93 | 7.2 |
| | 11481027 | 7.18 + 9.82→ 6.93 | 7.5 |
| | 11481059 | 7.26 + 5.76→ 7.05 | 7.9 |
| | 11481066 | 7.26 + 9.69→ 7.20 | 8.1 |
| Ca/F/ | 11000032 | 9.57 + 11.49 → 11.04 | 10.4 |
| Thioglycollate | | | |
| Ca/F/ | 11481198 | 9.83 + 9.31 → 9.12 | 8.1 |
| Hypoxanthine | | | |
| Ca/F/Xanthine | 11481199 | 10.75 + 9.39→9.05 | 10.1 |
| Ca/F/Guanine | 11481195 | 12.52 + 8.48→ 11.66 | 6.8 |
| Ca/F/Cytosine | 11954009 | 9.03 + 9.18 →6.92 | 7.6 |
| Ca/F/Thymine | 11954064 | 9.11 + 8.31→ 8.66 | 8.5 |

When carbonate was simultaneously used during CaF2 precipitation, Ca/F/CO3 composite particles were obtained. By varying the relative quantities of bicarbonate or carbonate, composites exhibiting different surface properties were obtained (Table 2B).

TABLE 2B
Comparison of E.C.P. values in the 8833172A-D series

| | HCO₃⁻/ F⁻/Ca⁺⁺ | % of CO₃ | E.C.P. | E.C.P. |
|---|---|---|---|---|
| Batches | mole ratio | by titration | H₂O/KCI | H₂O/KNO₃ |
| 8833172A | 2/36/20 | 3.5 | 6.4 and 8.7 | 8.7 |
| 8833172B | 1/38/20 | 1.1 | 6.3 and 8.4 | 8.4 |
| 8833172C | 0.2/39.6/20 | 0.09 | 5.1 and 9.1 | 7.0 |
| 8833172D | 0.0/40/20 | 0 | 7.8 | 6.6 |

For low carbonate molar ratios the formed composite exhibits carbonate of the vaterite type. This was a surprise since other described vaterite formations indicate that high concentrations (CaCl2 1M+K2CO3 1M) were needed to obtain vaterite type of carbonate Mori et al. (2009) Materials Science and Engineering 29:1409-1414 and Parkin et al. (2009) Optics Express 17:21944-21955. The vaterite type of carbonate obtained by the method disclosed herein is of importance for adsorption of organic material possessing immunological properties (see experimental part: adsorption of MPL).

When cysteine was used during precipitation, Ca/F/cysteine composite particles were obtained. By varying the order of addition, composites exhibiting different surface properties were obtained (Table 3).

TABLE 3
Precipitations in presence Cysteine

| | | | (CaCl₂ + Cys) | | NaF pH 8.98 + | | | |
|---|---|---|---|---|---|---|---|---|
| (NaF + Cys) pH 8.18 + | | | pH 8.18 + NaF | | (CaCl₂ + Cys) | | CaCl₂ pH 10.04 + | |
| CaCl₂ pH 10.03 | | | pH 8.78 | | pH 8.19 | | (NaF + Cys) pH 8.19 | |
| Batch # 9440055 | | | Batch # 9440056 | | Batch # 9440057 | | Batch # 9440058 | |
| Washing | pH | mOsm/kg | pH | mOsm/kg | pH | mOsm/kg | pH | mOsm/kg |
| W1 | 8.03 | 409 | 8.25 | 411 | 8.26 | 405 | 8.05 | 410 |
| W2 | 8.14 | 149 | 8.37 | 153 | 8.37 | 142 | 8.16 | 148 |
| W3 | 8.19 | 69 | 8.42 | 71 | 8.44 | 65 | 8.27 | 71 |
| W4 | 8.18 | 33 | 8.44 | 34 | 8.48 | 31 | 8.19 | 34 |
| W5 | 8.36 | 16 | 8.84 | 25 | 8.85 | 15 | 8.36 | 17 |
| W6 | 8.43 | 8 | 8.86 | 12 | 8.89 | 7 | 8.47 | 7 |
| W7 | 8.47 | 4 | 8.91 | 3 | 8.91 | 3 | 8.51 | 3 |
| W8 | 8.32 | 2 | 8.58 | 2 | 8.56 | 2 | 8.34 | 2 |
| W9 | 8.15 | 1 | 8.33 | 1 | 8.48 | 1 | 8.36 | 1 |
| W10 | 8.07 | 1 | 8.17 | 2 | 8.12 | 1 | 8.07 | 2 |

| | | | | |
|---|---|---|---|---|
| Conc. | 10.81 mg/ml | 13.20 mg/ml | 17.08 mg/ml | 8.72 mg/ml |
| E.C.P. | H₂O/KCl = 8.6 | H₂O/KCl = 8.8 | H₂O/KCl = 8.9 | H₂O/KCl = 8.7 |
| | H₂O/KNO₃ = 8.5 | H₂O/KNO₃ = 8.6 | H₂O/KNO₃ = 8.5 | H₂O/KNO₃ = 8.5 |
| μgN/ml | 193.5 | 292.8 | 412.9 | 187.1 |

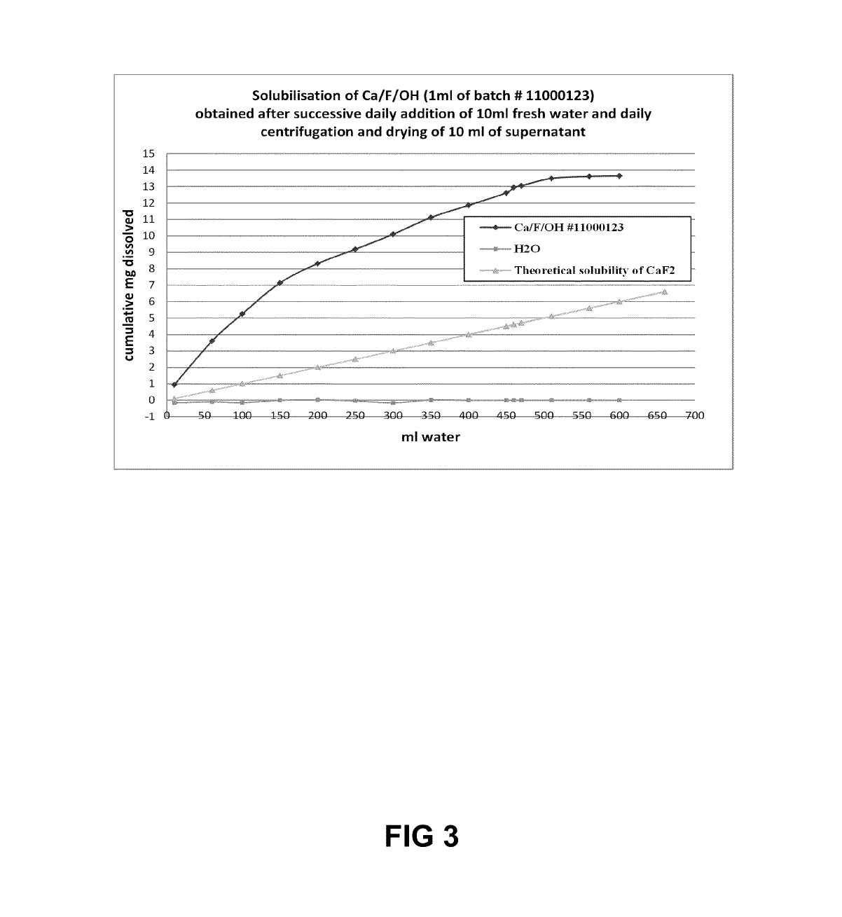

Nanoparticles obtained herein exhibit higher solubility compared to handbook standard values (which were generally related to mono-crystals). For example, FIG. 3 presents the water solubility of Ca/F/OH nanoparticles batch 11000123. This composite is more soluble compared to the solubility of CaF2 reported in handbooks (0.14 mM). Thus, these types of nano-composite particles are of great interest in the vaccine field using IM mode of administration.

Nitrogen content was analyzed by Antek as described in the Analytical Methods. From those results it is thought that a large majority of the nitrogen, originated from the selected starting organic material used during the preparation, is located on the insoluble particles (See Table 4A).

TABLE 4A
Nitrogen content by Antek analyses

| | | | Antek | | | |
|---|---|---|---|---|---|---|
| | | Theoretical | in | Total | mg dry | μgN/mg dry |
| | | μgN/ml | W10 | μgN/ | material/ | material |
| | Batch # | in W10 | μgN/ml | ml | ml | (£) |
| Ca/F/Cysteine | 9440055 | 15.6 | 35.1 | 193.5 | 10.8 | 14.6 |
| | 9440056 | 15.6 | 53.8 | 292.8 | 13.2 | 18.1 |
| | 9440057 | 15.6 | 42.3 | 412.9 | 17.08 | 21.7 |
| | 9440058 | 15.6 | 43.5 | 157.1 | 8.72 | 13.0 |
| | 9440099 | 17 | 8.5 | 726 | 20.84 | 34.4 |
| | 9440197 | 17 | 34.6 | 705.5 | 20.00 | 33.5 |
| Ca/F/N-Ac.Cysteine | 9440110 | 17 | 26 | 129 | 10.36 | 9.9 |
| | 9440196 | 17 | 22.6 | 112.9 | 9.34 | 9.6 |
| | 10616125 | 17 | 37.2 | 115.9 | 9.30 | 8.4 |
| | 11000101 | 18.2 | 11.7 | 129.1 | 14.9 | 7.88 |
| Ca/F/Glutathione | 10616185 | 32.8 | 44.1 | 2062.4 | 18.43 | 109.5 |
| | 11000030 | 5.1 | 41 | 2178 | 27.20 | 78.5 |
| | 11000033 | 4.4 | 55 | 2526 | 23.52 | 105.0 |
| | 11000086 | 4.8 | 14.1 | 2789.4 | 24.76 | 112.08 |
| | 11000099 | 4.5 | 139.5 | 2425.2 | 23.19 | 98.56 |
| | 11000194 | 4.1 | 6.3 | 2870 | 22.02 | 130 |
| Ca/F/Glutathione | 10616198 | 32.8 | 256 | 2226 | 11.0 | 179.1 |
| oxide | 11000139 | 3.6 | 70 | 4130 | 22.12 | 183.5 |
| | 11000140 | 7.3 | 90 | 4400 | 22.6 | 190.7 |
| Ca/F/Uric acid | 11000182 | 0.04 | 7.1 | 260.3 | 12.89 | 19.6 |
| Ca/F/Folic | 11418018 | 0.77 | 0 | 2034 | 15.02 | 135.4 |
| Ca/F/Hypoxanthine | 11481198 | | 95.9 | 314.2 | 13.1 | 23.98 |
| Ca/F/Xanthine | 11481199 | | 183.6 | 2185.2 | 20.3 | 107.6 |
| Ca/F/Guanine | 11481195 | | 196.4 | 1253 | 21.5 | 49.14 |
| Ca/F/Cytosine | 11954009 | | 4.9 | 0.3 | 13.7 | 0.02 |
| Ca/F/Thymine | 11954064 | | 0 | 58.3 | 9.8 | 5.95 |

(£) = (Total μgN/ml W10 μgN/ml)/mg dry material/ml
W10 is the supernatant coresponding to the water washing step number 10.

Antioxidant capacity was used to determine % w/w oxidizable organic material/dry weight as described in the Analytical Methods. The results are shown in Table 4B.

TABLE 4B
Anti-oxidant capacity of various CaF₂ based composites

| | | | Anti- | | Oxidizable | |
|---|---|---|---|---|---|---|
| | | | oxidant | capacity | organic content | |
| | | Suspension | μl | μl | | % w/w |
| | Batch | Concent. | KMnO₄/ | KMnO₄/ | μmoles | Org./dry |
| Suspension | # | mg dry/ml | ml susp. | mg dry | Org./ml | weight |
| CaF₂ | 8833190 | 33.2 | <50 | <1.5 | NA | NA |
| Ca/F/ | 9440099 | 20.8 | 7950 | 382 | 6.81 | 3.9 |
| Cysteine | 9440197 | 20.0 | 7926 | 396 | 6.72 | 4.0 |
| Ca/F/N- | 9440110 | 10.36 | 1843 | 177 | 1.75 | 2.4 |
| Acetyl- | 9440196 | 9.3 | 3236 | 239 | 3.03 | 5.3 |
| Cysteine | | | | | | |

### Example 2

### Formation of Composites

Various composites were formed according to the general schemes provided in the Detailed Description. Unless indicated to the contrary, all starting materials used herein were obtained commercially.

### 1° Treatment of Commercial Solid Particles

### {CaCO₃}Solid Water Washing: Batch #8833111

Calcium carbonate from queries: OMYA® 264.8 mg was treated with 250 ml of water as described (Scheme 1). Supernatant pH is given (see Table 5).

TABLE 5
pH along the water washing steps and addition of various solutions
on {CaCO₃}_{solid} particles

| | 8833111 | 8833167 | 11000077 | 8833107 | 9923127 | 8833164 | 8833110 | 8833139 |
|---|---|---|---|---|---|---|---|---|
| | | | | Starting pH | | | | |
| | | | | 7.43 | 7.48 | 7.48 | 8.18 | 8.11 |
| Washing | pH | pH | pH | pH | pH | pH | 8.22 | 8.41 |
| W1 | 9.80 | 9.43 | 10.01 | 11.08 | 11.12 | 7.22 | 8.24 | 8.62 |
| W2 | 9.66 | 9.33 | 9.92 | 11.01 | 11.14 | 7.04 | 8.29 | 8.8 |
| W3 | 9.72 | 9.51 | 9.79 | 10.88 | 10.94 | 7.24 | 8.42 | 8.93 |
| W4 | 9.67 | 9.43 | 9.72 | 10.71 | 10.83 | 8.25 | 8.52 | 9.08 |
| W5 | 9.78 | 9.36 | 9.71 | 10.47 | 10.68 | 8.01 | 8.69 | 9.27 |
| W6 | 9.71 | 9.47 | 9.70 | 10.21 | 10.44 | 8.92 | 8.8 | 9.44 |
| W7 | 9.78 | 9.45 | 9.66 | 9.8 | 10.22 | 9.02 | 9.11 | 9.72 |
| W8 | 9.82 | 9.57 | 9.57 | 9.57 | 9.83 | 9.74 | 9.32 | 9.84 |
| W9 | 9.68 | 9.52 | 9.66 | 9.41 | 9.65 | 9.64 | 9.44 | 9.85 |
| W10 | 9.45 | 9.45 | ND | 9.15 | 9.18 | 9.66 | 64% | 62% |
| Yields | 82.4% | 70.6% | 83.8% | 67% | 74.8% | 84% | 9.4 | 9.9 |
| E.C.P. | 9.3 | 9.5 | 9.8 | 9.0 | 7.4 and 9.2 | 9.6 | | |

| | | | |
|---|---|---|---|
| Elem. Analyses | ND | Ca: 48% | ND |
| | | F: 41.9% | |
| CO₃ by titration | ND | 2.6% | ND |

The final volume was 30 ml and E.C.P. was measured on this suspension. Ten ml of this suspension was evaporated to dryness at 80° C. and weighted. Yields were expressed in % compared to the weight of starting powder. Sample of dry material was submitted to infrared analysis which shows the calcite type of CaCO₃.

### {CaCO₃}Solid Water Washing: Batch #8833167

Synthetic calcium carbonate, precipitated by bubbling CO2 in a Ca(OH)2 solution, from Mineral Technology® (Multiplex MM batch U203) 247.9 mg was treated as batch #8833111 (Table 5).

### {CaCO₃}Solid Water Washing: Batch #11000077

High Gravity Controlled Precipitation NPCC111 from NanoMaterials Technology® synthetic calcium carbonate, 0.828 g was treated as batch #8833111 (Table 5).

{CaCO₃}Solid Treated with NaF Solution: Batch #8833107

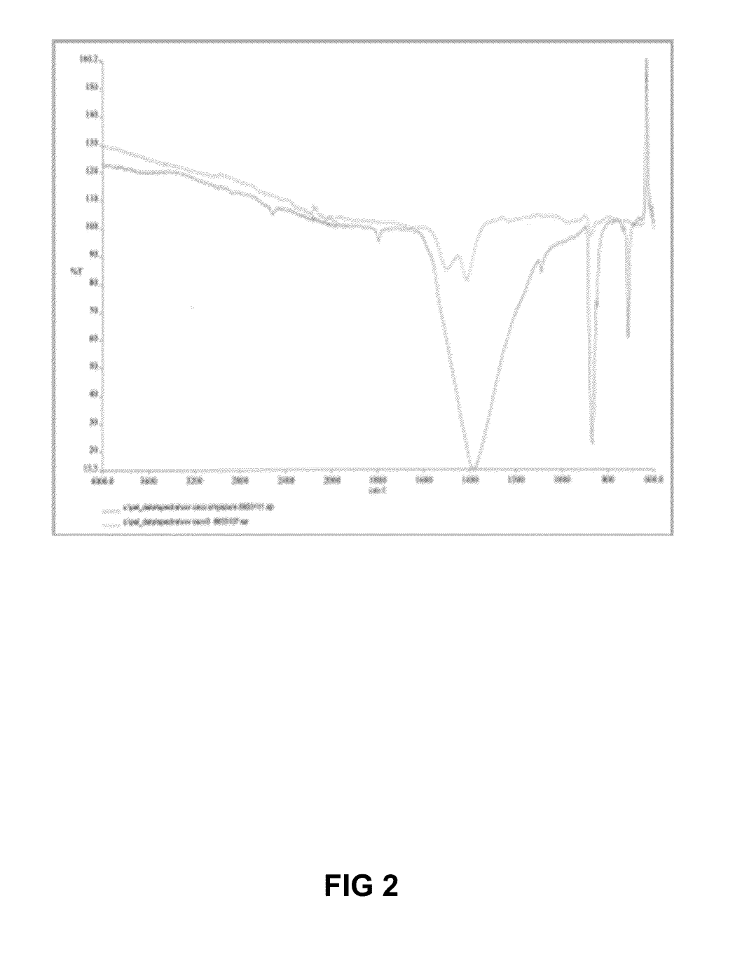

Sodium fluoride (710.7 mg) was dissolved in water and pH adjusted to pH 7.43, forming a total volume of 168 ml which was sterilized by filtration. To this solution, 265.1 mg of CaCO₃ solid particles (OMYA®) were added. Water washing was carried out as described (Scheme 2). Supernatant pH is given (see Table 5), final volume being 30 ml. Ten ml of this suspension was evaporated to dryness at 80° C. and weighted. Yields were expressed in % compared to the weight of starting powder. Sample of dry material was submitted to infrared analysis which shows the presence of CaCO₃ of the Vaterite type (FIG. 2).

{CaCO₃}Solid Treated with NaF Solution: Batch #9923127

2.6612 g of CaCO₃(OMYA®) was treated by 7.0843 g NaF dissolved in 1 Liter water, giving a starting pH of 9.52 and treated as for batch #8833107 (Table 5). The yields were 74.8%. Elementary Ca and F analyses were given (Table 5). Theoretical elementary composition of CaCO₃ is Ca: 40% and CO3: 60%; while CaF2 gives Ca: 51.28% and F: 48.72%. Experimental data give Ca: 48% and F:41.9% indicating that fluoride % is too low to be pure CaF2 and Ca % is too low to be pure CaCO3. Sample of dry material was submitted to infrared analysis showing the presence of carbonate of vaterite type similar to the one of sample #8833107 presented in FIG. 2. Titration by HCl indicates that this powder was only 2.6% carbonate. Thus, a composite of Ca/F/CO₃ was obtained in which the CaCO₃ part was of the vaterite type and thus differs from the starting CaCO₃ material.

{CaCO₃}Solid Treated with CaCl2 Solution: Batch #8833164

Calcium chloride (2.0223 g) was dissolved in water and pH adjusted to pH 7.48, forming a total volume of 180 ml which was sterilized by filtration. To this solution, 276.8 mg of CaCO₃ solid particles (OMYA®) was added. Water washing and treatment was carried out as for batch #8833107 (Table 5).

{CaCO₃}Solid Treated with Cysteine Solution: Batch #8833110

Cysteine (2.0111 g) was dissolved in water and pH adjusted to pH 8.18, forming a total volume of 168 ml which was sterilized by filtration. To this solution, 277.4 mg of CaCO₃ solid particles (OMYA®) was added. Water washing was carried out as described (Scheme 2). Supernatant pH is given (see Table 5).

{CaCO₃}Solid Treated with N-Acetyl-Cysteine Solution: Batch #8833139

N-Acetyl-cysteine (3.1058 g) was dissolved in water and pH adjusted to pH 8.11, forming a total volume of 180 ml which was sterilized by filtration. To this solution, 266.3 mg of CaCO₃ solid (Sigma-Aldrich was added. Water washing was carried out as described (Scheme 2). Supernatant pH is given (see Table 5).

{CaCO₃}Solid Treated with Thioglycerol Batch #8833114

Thioglycerol (1.5 ml) was dissolved in water and pH adjusted to pH 9.50, forming a total volume of 168 ml which was sterilized by filtration. To this solution, 263.5 mg (2.63 mmoles) of CaCO₃ solid (OMYA®) was added. Water washing was carried out as described (Scheme 2). Supernatant pH is given in Table 6.

TABLE 6
pH along the water washing steps after addition of various solutions
on {CaCO₃}_{solid} particles

| | 8833114 | 8833109 | 8833141 | 8833142 | 8833148 | 8833149 | 8833150 | 9923130 | 9923131 |
|---|---|---|---|---|---|---|---|---|---|
| Starting | 9.50 | 6.54 | | 8.15 | 8.20 | 8.26 | 11.54 | 6.55 | 9.44 |
| PH | | | | | | | | | |
| Washing | pH | pH | pH | pH | pH | pH | pH | pH | pH |
| W1 | 9.45 | 6.66 | 6.62 | 8.15 | 8.24 | 8.5 | 11.53 | 6.58 | 9.49 |
| W2 | 9.51 | 6.74 | 6.27 | 8.19 | 8.36 | 8.64 | 11.38 | 6.61 | 9.53 |
| W3 | 9.5 | 6.86 | 6.78 | 8.22 | 8.42 | 8.55 | 11.25 | 6.65 | 9.56 |
| W4 | 9.57 | 7.07 | 6.32 | 8.23 | 8.37 | 8.54 | 11.11 | 6.67 | 9.58 |
| W5 | 9.69 | 7.33 | 6.37 | 8.22 | 8.17 | 8.53 | 10.99 | 6.67 | 9.60 |
| W6 | 9.64 | 7.78 | 6.21 | 8.24 | 7.51 | 7.96 | 10.45 | 6.56 | 9.48 |
| W7 | 9.55 | 8.08 | 6.61 | 8.22 | 8.05 | 8.61 | 10.45 | 6.69 | 9.50 |
| W8 | 9.65 | 8.76 | 6.74 | 8.1 | 7.69 | 8.79 | 10.23 | 6.50 | 9.34 |
| W9 | 9.59 | 8.97 | 6.81 | 7.72 | 8.06 | 8.84 | 9.98 | 6.33 | 8.76 |
| W10 | 9.61 | 8.88 | 6.55 | 7.52 | 7.66 | 8.62 | 9.27 | 6.19 | 8.20 |
| Yields | 75% | 69% | 73% | 70% | 68% | 73% | 74% | 83% | 74% |
| E.C.P. | 9.6 | 9.45 | 6-8 | <4.5 | 7.4 | 5.6 | 9.2 | No | No |
| H₂O/KCl | | | | | | | | crossing | crossing |
| E.C.P. | | | 7.2 | 7.2 | 7.4 | 6.1 | 8.6 | 7.4 | 7.4 |
| H₂O/KNO3 | | | | | | | | | |

{CaCO₃}Solid Treated with Phosphoethanolamine Batch #8833109

Phosphoethanolamine (2.3582 g) was dissolved in water and pH adjusted to pH 6.54, forming a total volume of 168 ml which was sterilized by filtration. To this solution, 270.6 mg of CaCO₃ solid particles (OMYA®) was added. Water washing was carried out as described (Scheme 2). Supernatant pH is given (see Table 6).

### {CaF₂}Solid Water Washing: Batch #8833141

Calcium fluoride 316.0 mg was treated with 250 ml of water as described (Scheme 1). Supernatant pH is given (see Table 6).

{CaF₂}Solid Treated with Cysteine Solution: Batch #8833142

Cysteine (2.0735 g) (Merck) was dissolved in water and pH adjusted to pH 8.15, forming a total volume of 182 ml which was sterilized by filtration. To this solution, 353.1 mg of calcium fluoride was added. Water washing was carried out as described (Scheme 2). Supernatant pH is given (see Table 6).

{CaF₂}Solid Treated with N-Acetyl-Cysteine Solution: Batch #8833148

N-Acetyl-Cysteine (3.01924 g) was dissolved in water and pH adjusted to pH 8.20, forming a total volume of 180 ml which was sterilized by filtration. To this solution, 362.89 mg of calcium fluorite was added. Water washing was carried out as described (Scheme 2). Supernatant pH is given (see Table 6).

{CaF₂}Solid Treated with Carbonate at pH 8.26 Batch #8833149

Sodium bicarbonate (1.51058 g) was dissolved in 180 ml of water, a pH 8.28 was obtained. This solution was sterilized by filtration. To this solution, 377.99 mg of calcium fluoride was added. Water washing was carried out as described (Scheme 2). Supernatant pH is given (see Table 6).

{CaF₂}Solid Treated with Carbonate at pH 11.54 Batch #8833150

Sodium carbonate (1.88348 g) was dissolved in 180 ml of water, a pH 11.54 was obtained. This solution was sterilized by filtration. To this solution, 383.85 mg of calcium fluoride was added. Water washing was carried out as described (Scheme 2). Supernatant pH is given (see Table 6).

{CaF₂}Solid Treated with Phosphoethanolamine Batch #9923130

Phosphoethanolamine (2.3517 g) was dissolved in water and pH adjusted to pH 6.55, forming a total volume of 180 ml which was sterilized by filtration. To this solution, 351.41 mg of calcium fluoride was added. Water washing was carried out as described (Scheme 2). Supernatant pH and osmotic pressure is given (see Table 6).

{CaF₂}Solid Treated with Thioglycerol Batch #9923131

Thioglycerol (1.5 ml) was dissolved in water and pH adjusted to pH 9.44, forming a total volume of 180 ml which was sterilized by filtration. To this solution, 350.6 mg of calcium fluoride was added. Water washing was carried out as described (Scheme 2). Supernatant pH and osmotic pressure is given (see Table 6).

### 2° Sol-Gel Precipitations

Na₂HPO₄ Solution Added to CaCl₂ Solution Batch #391080

Calcium chloride dihydrate (1.8370 g) was dissolved in 900 ml of water giving a pH of 6.37. After sterilization by filtration, this solution was placed in 2 liters sterile Duran-Schott. Disodium hydrogenophosphate dihydrate (2.2250 g) was dissolved in 900 ml of water giving a pH of 9.32. After sterilization by filtration and under aseptic conditions, this solution is added to the CaCl2 solution. After overnight decantation 1500 ml of supernatant was discarded and replaced by 1500 ml of sterile water. Those washing were repeated 12 times. Finally the suspension was concentrated by centrifugation to a total volume of 200 ml. Those particles exhibit an E.C.P. H2O/KCL of 7.8 (see Table 7).

CaCl₂ Solution Added to Na₂HPO₄ Solution Batch 391082

Disodium hydrogenophosphate dihydrate (2.22382 g) was dissolved in 900 ml of water. After sterilization by filtration, this solution placed in 2 liters sterile Duran-Schott.Calcium chloride dihydrate (1.83972 g) was dissolved in 900 ml. After sterilization by filtration and under aseptic conditions, this solution was added to the disodium hydrogenophosphate. The following treatments were similar to batch #391080 (Table 7).

Na₂HPO₄ and Citrate Solution Added to CaCl₂ Solution Batch #391084

Calcium chloride dihydrate (1.8413 g) was dissolved in 900 ml. After sterilization by filtration, this solution was placed in 2 liters sterile Duran-Schott.Disodium hydrogenophosphate dihydrate (2.2247 g) was dissolved in 900 ml of water and sterilized by filtration. Tri-sodium citrate (4.0259 g) was dissolved in 180 ml of water giving a pH of 8.49 and sterilized by filtration. Under aseptic condition the citrate solution was added to the disodium hydrogenophosphate solution and this mix was added to the CaCl2 solution. The following treatments were similar to batch #391080 (Table 7).

Na₂HPO₄ and Lysine Solution Added to CaCl₂ Solution Batch #391086

Calcium chloride dihydrate (1.8350 g) was dissolved in 900 ml. After sterilization by filtration, this solution was placed in 2 liters sterile Duran-Schott. Disodium hydrogenophosphate dihydrate was dissolved in 900 ml of water and sterilized by filtration. To 100 ml of water was added to 15 g of Lysine base.

Hydrochloric acid (0.1N) was added (40 ml) to obtain a pH of 10.1. This solution was sterilized by filtration and added to the disodium hydrogenophosphate solution and this mix was added to the CaCl2 solution. The following treatments were similar to hatch #391080 (Table 7)

TABLE 7
Influence of soluble substance during the precipitation of calcium
phosphate particles

| | | E.C.P. |
|---|---|---|
| Starting compounds | Batch # | H₂O/KCI |
| Na₂HPO₄ solution added to CaCl₂ solution and washed | 391080 | 7.8 |
| with water | | |
| CaCl₂ solution added to Na₂HPO₄ solution and washed | 391082 | 7.5 |
| with water | | |
| Na₂HPO₄ + Citrate solution added to CaCl₂ solution and | 391084 | 9.2 |
| washed with water | | |
| Na₂HPO₄ + Lysine solution added to CaCl₂ solution and | 391086 | 8.4 |
| washed with water | | |

### CaF₂ Batch #8833172D

Sodium fluoride (8.4158 g) was dissolved in 500 ml of water and adjusted to pH 7.25. The solution was sterilized by filtration and 100 ml of this solution was placed in a sterile 250 ml Duran-Schott flask.

Calcium chloride (13.3555 g) was dissolved in 600 ml of water and adjusted to pH 7.07. The solution was sterilized by filtration and 100 ml of this solution added to the NaF solution. Water washing were carried out according to Scheme 3. Supernatant pH is given (see Table 8).

### CaF₂ Batch #8833190

Sodium fluoride (8.4231 g) was dissolved in 500 ml of water. The solution was sterilized by filtration and placed in a sterile 1 liter Duran-Schott flask. Calcium chloride (11.1093 g) was dissolved in 500 ml of water. The solution was sterilized by filtration and added to the NaF solution. Water washing were carried out according to Scheme 3. Supernatant pH is given in Table 8.

TABLE 8
Sol-gel precipitation of various Calcium fluoride composites.

| | Precipitations in presence of | | | | | | |
|---|---|---|---|---|---|---|---|
| | decreasing bicarbonate quantities | | | | Precipitation of Ca/F/OH | | |
| | HCO₃⁻/F⁻/Ca⁺⁺ molar ratio: | | | | F/Ca molar ratio: | | |
| | 2/36/20 | 1/38/20 | 0.2/39.6/20 | 0.0/40/20 | 2/1 | 2/1 | 1/1 |
| | 8833172 A | 8833172 B | 8833172 C | 8833172 D | 8833190 | 9440194 | 11000123 |
| Starting pH | 8.53 + 8.07 | 8.44 + | 8.21 + | 7.37 + | | | |
| | | 8.07 | 8.07 | 8.07 | | | |
| Washing | pH | pH | pH | pH | pH | pH | pH |
| W1 | 6.48 | 6.29 | 6.09 | 5.93 | 9.44 | 9.78 | 5.93 |
| W2 | 6.8 | 6.71 | 6.63 | 6.1 | 7.15 | 8.82 | 6.1 |
| W3 | 7.17 | 7.16 | 7.07 | 6.24 | 6.36 | 7.13 | 6.24 |
| W4 | 7.23 | 7.27 | 7.16 | 6.13 | 6.47 | 6.78 | 6.13 |
| W5 | 7.57 | 7.57 | 7.41 | 6.34 | 6.38 | 6.52 | 6.34 |
| W6 | 7.54 | 7.48 | 7.18 | 6.14 | 6.27 | 6.31 | 6.14 |
| W7 | 7.47 | 7.28 | 6.86 | 5.91 | 6.36 | 6.25 | 5.91 |
| W8 | 7.38 | 7.17 | 6.84 | 6.09 | 6.25 | 6.18 | 6.09 |
| W9 | 7.43 | 7.32 | 6.99 | 6.15 | 6.16 | 6.44 | 6.15 |
| W10 | 6.98 | 6.9 | 6.6 | 5.83 | 6.02 | 6.08 | 5.83 |
| Conc. | 8.287 mg/ml | 9.377 | 18.52 | 25.274 | 33.21 | 26.3 | 14.25 |
| CO₃ | 3.54% | 1.13% | 0.09% | 0% | NA | NA | NA |
| E.C.P. | 8.7 | 8.4 | 9.1 | 7.8 | 0.8M = | 0.8M = | H₂O/KCl = |
| H₂O/KCl | | | | | 7.3 | 7.6 | 4.8 & |
| | | | | | 3.2M = | | 9.7 |
| | | | | | 8.3 | | |
| E.C.P. | 8.7 | 8.4 | 7.0 | 6.6 | 3.2M = | 3.2M = | 7.5 |
| H₂O/KNO3 | | | | | 6.9 | 6.4; | |
| | | | | | | 0.8M = | |
| | | | | | | 6.6 | |
| Elementary | | | | | Ca: | Ca: | Ca: 49.7% |
| analyses | | | | | 52.8% | 55.2% | F: 46.4% |
| | | | | | F: 46.6% | F: 44.7% | |

### CaF₂ Batch #9440194

Sodium fluoride (8.4273 g) was dissolved in 500 ml of water. The solution was sterilized by filtration and placed in a sterile 1 liter Duran-Schott flask. Calcium chloride (11.1174 g) was dissolved in 500 ml of water. The solution was sterilized by filtration and added to the NaF solution. Water washing were carried out according to Scheme 3. Supernatant pH is given in Table 8.

### Ca/F/OH Batch #11000123

Sodium fluoride (4.2055 g) was dissolved in 504.2 ml of water the pH was 9.40. The solution was sterilized by filtration and placed in a sterile 1 liter Duran-Schott flask. Calcium chloride (11.1107 g) was dissolved in 508.2 ml of water. The solution was sterilized by filtration and added to the NaF solution. Water washing were carried out according to Scheme 3. Supernatant pH is given in Table 8.

### Ca/F/CO₃ in the Carbonate Domain Batches 8833152 to 8833157

Na2CO₃ solution: 10.6031 g was dissolved in 500 ml of water (obtained pH 11.61) and sterilized by 0.2 μm filtration. NaF solution: 8.41413 g was dissolved in 500 ml water (obtained pH 9.66) and sterilized by 0.2 μm filtration. CaCl2 solution: 13.3250 g was dissolved in 600 ml water (obtained pH 10.07) and sterilized by 0.2 μm filtration.

Starting with those solutions the Ca/F/CO₃ batches 8833152 to 8833157 were carried out by following steps 1-3 according to table 9.

TABLE 9
Precipitation in presence of carbonate

| | Step 1 | Step 2 | Obtained | Step 3 | mmole ratio | | |
|---|---|---|---|---|---|---|---|
| Batch # | Na₂CO₃ | NaF | pH | CaCl₂ | CO₃ | F | Ca |
| 8833152 | 90 ml | 10 ml | 11.57 | 100 ml | 18 | 4 | 20 |
| 8833153 | 70 ml | 30 ml | 11.52 | 100 ml | 14 | 12 | 20 |
| 8833154 | 50 ml | 50 ml | 11.44 | 100 ml | 10 | 20 | 20 |
| 8833155 | 30 ml | 70 ml | 11.34 | 100 ml | 6 | 28 | 20 |
| 8833156 | 10 ml | 90 ml | 11.08 | 100 ml | 2 | 36 | 20 |
| 8833157 | 5 ml | 95 ml | 10.96 | 100 ml | 1 | 38 | 20 |

Water washing were carried out according to Scheme 6. Supernatant pH is given in Table 10.

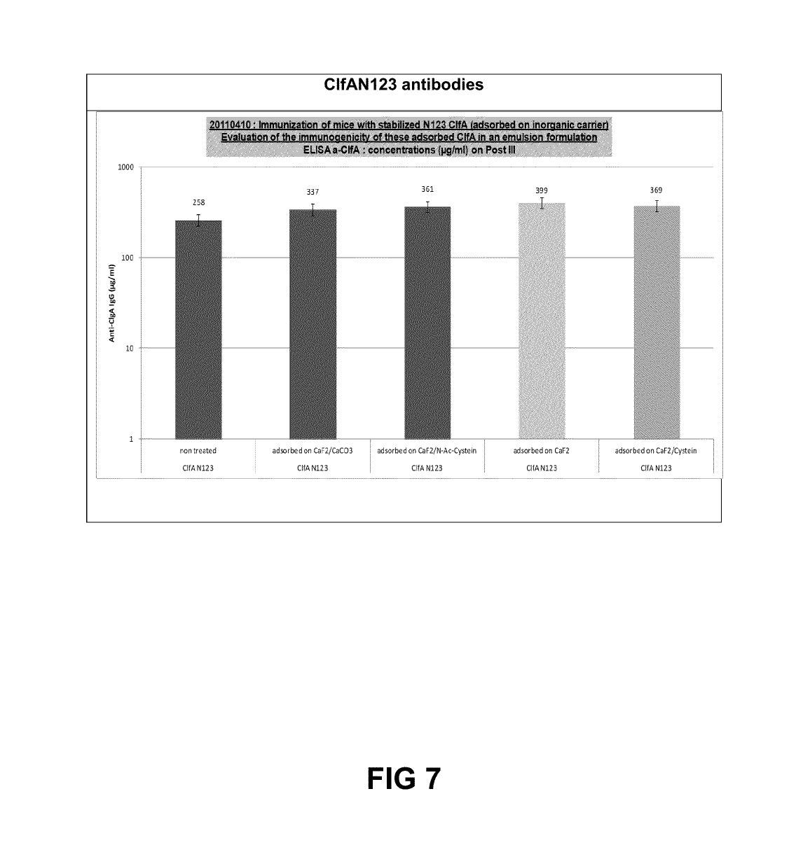

TABLE 10
Precipitation of Ca/F/CO₃ in the carbonate domain

| | CO₃⁻⁻/F^{-/}Ca⁺⁺molar ratio | | | | | |
|---|---|---|---|---|---|---|
| | Molar ratio: | | | | | |
| | 18/04/20 | 14/12/20 | 10/20/20 | 6/28/20 | 2/36/20 | 1/38/20 |
| | Batch # | | | | | |
| | 8833152 | 8833153 | 8833154 | 8833155 | 8833156 | 8833157 |
| Starting | 11.57 + | 11.52 + | 11.44 + | 11.34 + | 11.08 + | 10.93 + |
| pH | 10.07 | 10.07 | 10.07 | 10.07 | 10.07 | 10.07 |
| Washing | pH | pH | pH | pH | pH | pH |
| W1 | 9.66 | 9.89 | 10.19 | 10.24 | 9.8 | 10.19 |
| W2 | 9.55 | 9.98 | ND | 10.29 | 9.87 | ND |
| W3 | 9.55 | 9.96 | 10.15 | 10.18 | 9.81 | 10.15 |
| W4 | 9.55 | 9.82 | 10.07 | 10.05 | 9.74 | 10.07 |
| W5 | 8.81 | 9.09 | 9.52 | 9.53 | 9.22 | 9.52 |
| W6 | 9.45 | 9.49 | 9.68 | 9.76 | 9.58 | 9.68 |
| W7 | 9.48 | 9.52 | 9.61 | 9.74 | 9.54 | 9.61 |
| W8 | 9.43 | 9.46 | 9.57 | 9.67 | 9.5 | 9.57 |
| W9 | 8.87 | 8.57 | 8.89 | 9.13 | 9.16 | 8.89 |
| W10 | 9.31 | 9.24 | 9.4 | 9.42 | 9.33 | 9.4 |
| Conc. | 45.244 mg/ml | 41.739 mg/ml | 40.534 mg/ml | 42.726 mg/ml | 39.255 mg/ml | 16.007 mg/ml |
| % CO₃ | 86 | ND | 75.8 | 49.1 | 11.4 | 4.5 |
| IR | FIG. 7 | FIG. 7 | FIG. 7 | FIG. 7 | FIG. 7 | FIG. 7 |
| E.C.P./ | 9.2 | 9.3 | 9.3 | 9.4 | 9.3 | 8.9 |
| KCl | | | | | | |
| E.C.P./ | 9.4 | 9.3 | 9.4 | 9.3 | 9.1 | 8.5 |
| KNO3 | | | | | | |

Thus, decreasing CO₃− concentrations (or increasing the F− concentrations) gives less and less yields quantities, and lower W10 pH values.

Samples obtained at the highest F− concentration (#8833157) give the lowest E.C.P. value. This suggests that those obtained precipitates consist to CaCO₃ particles possibly containing increasing CaF₂ content as the F− concentrations were increased.

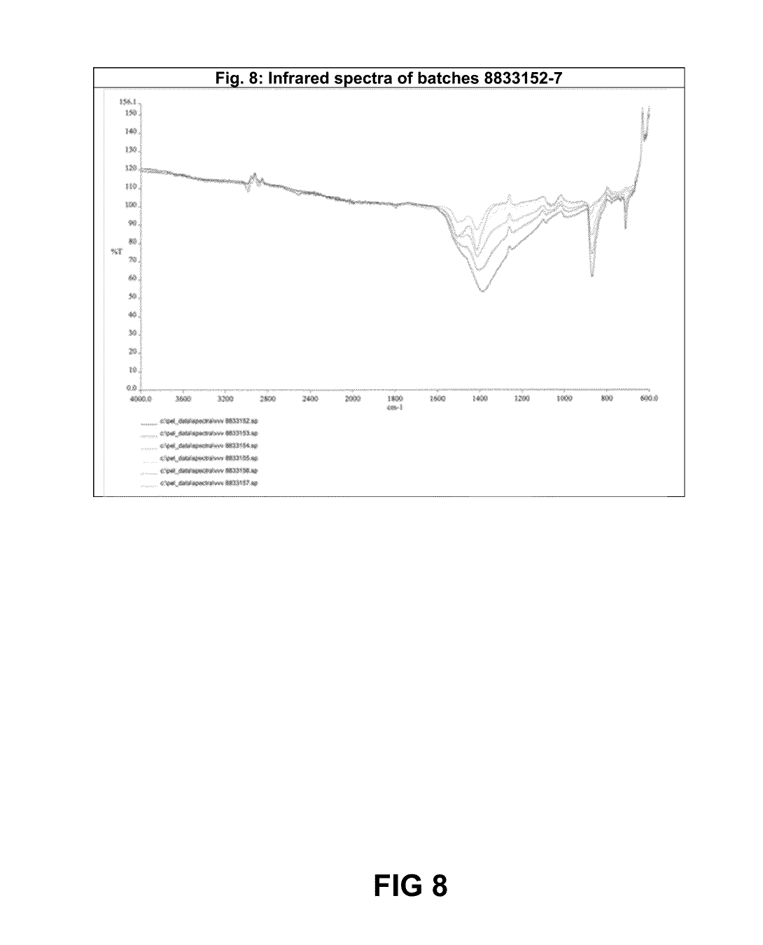

Thus, in the series Ca/F/CO₃8833152→8833157 we have:
less and less CO₃−, compensated by more and more F− (by starting mole ratio);
similar concentrations in terms of dry weight mg/ml (except 8833157 which was much lower);
decrease in CO₃− contents (confirmed by titration and by IR (870 cm-1 see FIG. 8);
decrease of the calcite form (IR 1430 cm-1 see FIG. 8) in favor of the vaterite one (IR 1490 and 1420 cm-1 see FIG. 8); #8833155, #8833156 and #8833157 were rich in Vaterite type; #8833152 was poor in Vaterite and rich in Calcite. Vaterite gives a weak IR signal at 750 cm-1 and no signal at 713-715 cm-1, while calcite exhibits IR signal at 713-715 cm-1 and no IR signal at 750 cm-1 (M. Sato and S. Matsuda; Zeitschrift für Kristallographie, 1969 vol. 129 p. 405-410);
similar E.C.P. H₂O/KCl values (except 8833157 which is lower);
decrease in E.C.P. H₂O/KNO₃ values at lower carbonate content.

### Ca/F/CO₃ in the Bicarbonate Domain:

Sodium bicarbonate solution: Sodium bicarbonate (8.4098 g) was dissolved in 500 ml of water (at this stage pH was 8.14) and sterilized by filtration. Sodium fluoride solution: Sodium fluoride (8.4158 g) was dissolved in 500 ml of water and the pH adjusted to 7.25. The solution was sterilized by filtration. Calcium chloride solution:Calcium chloride (13.3555 g) was dissolved in 600 ml of water and the pH adjusted to 8.07. The solution was sterilized by filtration

### Ca/F/CO₃ in the Bicarbonate Domain Batch #8833172A

Ten ml of sodium bicarbonate solution was placed in a sterile 250 ml Duran-Schott flask and 90 ml of the sodium fluoride solution was added, at this stage the pH was 8.53.

The calcium chloride solution (100 ml) was added to the NaHCO₃ and NaF solutions. Water washing were carried out according to Scheme 4. Supernatant pH is given in Table 8.

### Ca/F/CO₃ in the Bicarbonate Domain Batch #8833172B

5 ml of sodium bicarbonate solution was placed in a sterile 250 ml Duran-Schott flask and 95 ml of the sodium fluoride solution was added, at this stage the pH was 8.44.

The calcium chloride solution (100 ml) was added to the NaHCO₃ and NaF solutions. Water washing were carried out according to Scheme 4. Supernatant pH is given in Table 8.

### Ca/F/CO₃ in the Bicarbonate Domain Batch #8833172C

One ml of sodium bicarbonate solution was placed in a sterile 250 ml Duran-Schott flask and 99 ml of the sodium fluoride solution was added, at this stage the pH was 8.21.

The calcium chloride solution (100 ml) was added to the NaHCO₃ and NaF solutions. Water washing were carried out according to Scheme 4. Supernatant pH is given in Table 8.

HCl titrations carried out on batches 8833172A, 8833172B, 8833172C and 8833172D.

Presence of carbonate can be monitored by HCl titration. Comparisons were made by submitting similar quantities of nanoparticles, for example: 3.0 ml of 8833172A (at 8.28 mg/ml), 2.7 ml of 8833172B (at 9.37 mg/ml), 1.36 ml of 8833172C (at 18.52 mg/ml) and 1 ml of 8833172D (at 25.27 mg/ml), diluted when necessary in water to be at a total volume of 3 ml each, and titrated by HCl 0.3N solution (table 8).

### Ca/F/CO₃ Batch #9440195

Sodium bicarbonate (8.4109 g) was dissolved in 500 ml of water (at this stage pH was 8.17) and sterilized by filtration. Sodium fluoride (8.4203 g) was dissolved in 500 ml of water and the pH adjusted to 7.29. The solution was sterilized by filtration. Under aseptic conditions, 50 ml of the bicarbonate solution and 450 ml of the sodium fluoride solution was placed in a sterile 1 L Duran-Schott. Calcium chloride (11.1089 g) was dissolved in 500 ml of water and the pH adjusted to 7.96. The solution was sterilized by filtration and added to the Duran-Schott container. Water washing were carried out according to Scheme 4. Supernatant pH and results of HCl titrations are given (Table 11).

TABLE 11
Precipitation of Ca/F/CO₃

| | HCO₃⁻/F⁻/Ca⁺⁺ molar ratio | | | CO₃⁻⁻/F⁻/Ca⁺⁺ molar ratio | | | |
|---|---|---|---|---|---|---|---|
| | 2/36/20 | | | 18.3/4/20 | 10.2/20/20 | 6.1/28/20 | 2/36/20 |
| | 9440195 | 9923123 | 9923124 | 11000080 | 11000081 | 11000082 | 11000083 |
| | Starting pH | | | Starting pH | | | |
| HCO₃⁻ | 8.17 | 8.12 | 8.12 | 10.09 | 10.07 | 10.06 | 10.05 |
| F⁻ | 7.29 | 7.25 | 7.27 | | | | |
| Ca⁺⁺ | 7.96 | 7.72 | 7.70 | 10.17 | 10.17 | 10.17 | 10.17 |
| Washing | pH | pH | pH | pH | pH | pH | pH |
| W1 | 6.38 | 6.43 | 6.46 | 7.55 | 7.41 | 7.92 | 7.68 |
| W2 | 6.78 | 6.86 | 6.98 | 6.93 | 7.39 | 8.18 | 8.03 |
| W3 | 7.13 | 7.12 | 7.34 | 7.08 | 7.73 | 8.38 | 7.84 |
| W4 | 7.43 | 7.54 | 7.65 | 7.17 | 8.04 | 8.78 | 7.49 |
| W5 | 7.46 | 7.63 | 7.80 | 7.69 | 8.45 | 8.87 | 7.45 |
| W6 | 7.48 | 7.85 | 7.81 | 8.15 | 8.88 | 9.07 | 8.46 |
| W7 | 7.54 | 7.87 | 7.81 | 8.52 | 9.27 | 9.33 | 8.19 |
| W8 | 7.56 | 7.74 | 7.70 | 8.74 | 9.39 | 9.34 | 8.28 |
| W9 | 7.53 | 7.87 | 7.85 | 9.10 | 9.54 | 9.40 | 8.23 |
| W10 | 7.64 | 7.98 | 7.91 | 9.32 | 9.60 | 9.57 | 8.55 |
| Conc. | 14.6 mg/ml | 16.27 | 19.67 | 110.28 | 40.79 | 33.73 | 23.56 |
| Ca % F % | 53.2 | 51.0 | 49.0 | ND | 45.1 39.5 | 47.3 41.3 | 48.8 46.3 |
| | 46. | 42.1 | 43.0 | | | | |
| E.C.P./KCl | 9.3 | 9.2 | 9.5 | ND | 10.0 | 10.0 | 9.8 |
| E.C.P.KNO₃ | 8.6 | 8.3 | 8.3 | ND | 9.6 | =9.5 | =9.3 |
| % CO₃ | 3.6% | 2.9% | 2.9% | ND | 44.25 | 23.40 | 6.15 |

### Ca/F/CO₃ Batch #9923123

Sodium bicarbonate (8.4122 g) was dissolved in 500 ml of water (at this stage pH was 8.12) and sterilized by filtration. Fifty ml of this solution was placed in a sterile 1 liter Duran-Schott flask. Sodium fluoride (8.42488 g) was dissolved in 500 ml of water and the pH adjusted to 7.25. The solution was sterilized by filtration and 450 ml was added to the 1 liter Duran-Schott flask containing already the bicarbonate solution. Calcium chloride (11.1275 g) was dissolved in 500 ml of water and the pH adjusted to 7.72. The solution was sterilized by filtration and added to the NaHCO₃ and NaF solutions. Water washing were carried out according to Scheme 4. Supernatant pH and results of HCl titrations: see Table 11.

### Ca/F/CO₃ #9923124

50 ml of the sodium bicarbonate solution prepared for batch 9923123 was placed in a 1 liter sterile Duran-Schott flask. Sodium fluoride (8.42253 g) was dissolved in 500 ml of water and the pH adjusted to 7.27. The solution was sterilized by filtration and 450 ml was added to the 1 liter Duran-Schott flask containing already the bicarbonate solution.

Calcium chloride (11.1111 g) was dissolved in 500 ml of water and the pH adjusted to 7.70. The solution was sterilized by filtration and added to the NaHCO₃ and NaF solutions. Water washing were carried out according to Scheme 4. Supernatant pH and results of HCl titrations were given (Table 11).

### Ca/F/CO₃ in the Carbonate Domain 11000080-83

Sodium carbonate solution: Sodium bicarbonate (17.11 g) was dissolved in 1000 ml of water and NaOH was added to reach pH 10.09. This solution was sterilized by filtration. Sodium fluoride solution: Sodium fluoride (20.16 g) was dissolved in 1200 ml of water and the pH was 9.84. The solution was sterilized by filtration. CaCl₂ solution: 44.4 g was dissolved in 2000 ml water (obtained pH 10.17) and sterilized by 0.2 μm filtration.

### Ca/F/CO₃ 11000080:

Using a 1 L sterile Duran-Schott: 450 ml of the carbonate solution was added to 50 ml of sodium fluoride solution, resulting in a pH 10.09. Under laminar flow, 500 ml of the calcium chloride solution was added. Water washing were carried out according to Scheme 4. Supernatant pH is given in Table 11.

### Ca/F/CO₃ 11000081:

Using a 1 L sterile Duran-Schott: 250 ml of the carbonate solution was added to 250 ml of sodium fluoride solution, resulting in a pH 10.07. Under laminar flow, 500 ml of the calcium chloride solution was added. Water washing were carried out according to Scheme 4. Supernatant pH is given in Table 11.

### Ca/F/CO₃ 11000082:

Using a 1 L sterile Duran-Schott: 150 ml of the carbonate solution was added to 350 ml of sodium fluoride solution, resulting in a pH 10.06. Under laminar flow, 500 ml of the calcium chloride solution was added. Water washing were carried out according to Scheme 4. Supernatant pH is given in Table 11.

### Ca/F/CO₃ 11000083:

Using a 1 L sterile Duran-Schott: 50 ml of the carbonate solution was added to 450 ml of sodium fluoride solution, resulting in a pH 10.05. Under laminar flow, 500 ml of the calcium chloride solution was added. Water washing were carried out according to Scheme 8. Supernatant pH is given in Table 19.

### Ca/F/Ascorbic Acid Batch #9440198

Calcium ascorbate (42.6001 g) was dissolved in 400 ml of water (obtained pH=7.35). Sodium hydroxide 17 ml (0.5M) was added to reach pH8.99. Water was added (83 ml) giving a pH 9.08 and this solution was sterilized by filtration and placed in a sterile 1 liter Duran-Schott flask. Sodium fluoride (4.2063 g) was dissolved in 500 ml water (obtained pH 9.49) and sterilized by filtration. This solution was added to the calcium ascorbate solution. The following day the mix was transferred in a 2 L sterile Duran Schott flask and an additional 1 liter sterile water was added. An additional 15 days standing period was applied. Water washing were carried out according to Scheme 7. Supernatant pH is given in Table 12.

TABLE 12
precipitation of Calcium fluoride Z composites

| | Batch # | | | | | | |
|---|---|---|---|---|---|---|---|
| | 9440198 | 9440099 | 9440197 | 9440110 | 9440196 | 10616125 | 11000101 |
| Starting | 9.08 | 9.22 | 9.00 8.96 | 9.26 + | 9.66 + | 9.01 + | 9.35 + |
| pH | 9.49 | 8.22 | | 8.35 | 8.35 | 8.30 | 8.36 |
| Washing | pH | pH | pH | pH | pH | pH | pH |
| W1 | 6.65 | 8.27 | 8.31 | 8.47 | 8.44 | 8.44 | 8.47 |
| W2 | 6.67 | 8.45 | 8.41 | 8.62 | 8.58 | 8.54 | 8.62 |
| W3 | 6.65 | 8.52 | 8.50 | 8.76 | 8.72 | 8.72 | 8.76 |
| W4 | 6.67 | 8.60 | 8.65 | 8.92 | 8.88 | 8.87 | 8.89 |
| W5 | 6.77 | 8.75 | 8.78 | 9.15 | 9.10 | 9.08 | 8.98 |
| W6 | 6.64 | 8.97 | 9.03 | 9.43 | 9.38 | 9.34 | 9.02 |
| W7 | 6.89 | 9.21 | 9.18 | 9.63 | 9.47 | 9.45 | 9.20 |
| W8 | 6.86 | 9.22 | 9.18 | 9.47 | 9.15 | 8.93 | 8.64 |
| W9 | 7.02 | 9.12 | 9.02 | 9.35 | 8.31 | 7.67 | 7.85 |
| W10 | 7.12 | 8.96 | 8.64 | ND | 7.05 | 7.26 | 7.37 |
| Conc. | 13.5 mg/ml | 20.8 mg/ml | 20.0 mg/ml | 10.3 mg/ml | 9.34 mg/ml | 9.30 mg/ml | 14.9 mg/ml |
| Ca % F % | 52.9 | 54.2 | 55.1 | 49.4 | 51.1 | 49.8 35.3 | 51.5 54.8 |
| | 46.8 | 45.0 | 44.8 | 50.0 | 48.5 | | |
| E.C.P. | 5.7 & 7.2 | 9.3 | 9.6 | 7.5 | 8.3 | 7.8 | 6.1 & |
| H₂O/KCl | & 7.8 | | | | | | 10.0 |
| E.C.P. | 7.3 | 8.6 | 8.4 | 7.3 | 7.3 | 7.3 | 5.4 & 9.4 |
| H₂O/KNO₃ | | | | | | | |
| μgN/ml | NA | 726 | 706 | 129 | 113 | 115.9 | 129.1 |

### Ca/F/Cysteine Batch #944055

Sodium fluoride 0.84108 g and cysteine 2.42216 g (Merck) was dissolved in 84 ml of water. Sodium hydroxide 0.5N (16 ml) was added to reach pH 8.18. The solution was sterilized by filtration and placed in a 250 ml sterile Duran-Schott flask. Calcium chloride 2.22197 g was dissolved in 100 ml of water resulting in a pH 10.03 solution which was sterilized by filtration. Under sterile conditions this CaCl₂ solution was added to the (NaF+Cysteine) solutions. Water washing and supernatant pH are given in Table 3.

### Ca/F/Cysteine Batch #944056

Calcium chloride 2 and cysteine 2.42838 g (Merck) was dissolved in 80 ml of water. Sodium hydroxide 0.5N (21 ml) was added to reach pH 8.18. The solution was sterilized by filtration and placed in a 250 ml sterile Duran-Schott flask. Sodium fluoride 0.84168 g was dissolved in 100 ml of water resulting in a pH 8.78 solution which was sterilized by filtration. Under sterile conditions this NaF solution was added to the (CaF₂+Cysteine) solution. Water washing, and supernatant pH are given (Table 3).

### Ca/F/Cysteine Batch #944057

Sodium fluoride 0.84196 g was dissolved in 100 ml of water resulting in a pH 8.98 solution which was sterilized by filtration and placed in a 250 ml sterile Duran-Schott flask. Calcium chloride 2.2265 g and cysteine 2.42194 g (Merck) was dissolved in 80 ml of water. Sodium hydroxide 0.5N (21 ml) was added to reach pH 8.19. The solution was sterilized by filtration. Under sterile conditions this (CaF₂+Cysteine) solution was added to the NaF solutions. Water washing and supernatant pH are given (Table 3).

### Ca/F/Cysteine Batch #944058

Calcium chloride 2.2242 g was dissolved in 100 ml of water resulting in a pH 10.04 solution which was sterilized by filtration and placed in a 250 ml sterile Duran-Schott flask. Sodium fluoride 0.84254 g and cysteine 2.42793 g (Merck) was dissolved in 83.5 ml of water. Sodium hydroxide 0.5N (16.5 ml) was added to reach pH 8.19. The solution was sterilized by filtration. Under sterile conditions this (NaF+Cysteine) solution was added to the CaF₂ solutions. Water washing and supernatant pH: see Table 3.

### Ca/F/Cysteine Batch #9440099

Sodium fluoride (4.2287 g) was dissolved in 500 ml of water, pH at this stage was 9.22, and sterilized by filtration and placed in a sterile 1 liter Duran-Schott flask. Calcium chloride (11.1582 g) and cysteine (12.1086 g) (Merck) was added and dissolved in 400 ml of water (slightly violet color was obtained and pH=5.58). Sodium hydroxide 0.5N was added (117 ml) to reach pH 8.22 and this slightly violet solution was sterilized by filtration. The CaCl2-cysteine solution was added to the NaF solution. Water washing were carried out according to Scheme 5. Supernatant pH and osmotic pressure is given (see Table 12).

### Ca/F/Cysteine Batch #9440197

Sodium fluoride (4.215 g) was dissolved in 400 ml of water (obtained pH=9.62), sterilized by filtration and placed in a sterile 1 liter Duran-Schott flask. Calcium chloride (11.1037 g) was dissolved in 400 ml of water (obtained pH=10.06) and cysteine (12.1060 g) (Merck) was added (slightly violet color was obtained pH=5.6). Sodium hydroxide 0.5N was added (107 ml) to reach pH 8.21 and this slightly violet solution was sterilized by filtration. The CaCl2-cysteine solution was added to the NaF solution. Water washing were carried out according to Scheme 5. Supernatant pH is given in Table 12).

### Ca/F/N-Acetyl-Cysteine Batch #9440110

Sodium fluoride (4.2058 g g) was dissolved in 400 ml of water (obtained pH=9.26), and sterilized by filtration and placed in a sterile 1 liter Duran-Schott flask. Calcium chloride (11.10976 g) was dissolved in 400 ml of water and N-Acetyl-Cysteine (16.37876 g) was added (obtained pH=1.67). Sodium hydroxide was added (0.550 g) and sodium hydroxide 0.5N was added (196 ml) to reach pH 8.35 and this slightly violet solution (coloration appears above pH 5) was sterilized by filtration. The CaCl2-N-acetyl-cysteine solution was added to the NaF solution. Water washing were carried out according to Scheme 5. Supernatant pH is given in Table 12.

### Ca/F/N-Acetyl-Cysteine Batch #9440196

Sodium fluoride (4.2026 g) was dissolved in 400 ml of water (obtained pH=9.66), and sterilized by filtration and placed in a sterile 1 liter Duran-Schott flask. Calcium chloride (11.1139 g) was dissolved in 400 ml of water (obtained pH=10.04) and N-Acetyl-Cysteine (16.3025 g) was added (obtained pH=1.4). Sodium hydroxide (solid pellets Merck product 1064981000 batch 60467298) was added (0.848 g) and sodium hydroxide 0.5N was added (172 ml) to reach pH 8.35 and this slightly violet solution (coloration appears above pH 5) was sterilized by filtration. The CaCl2-N-acetyl-cysteine solution was added to the NaF solution. Water washing were carried out according to Scheme 5. Supernatant pH is given in Table 12).

### Ca/F/N-Acetyl-Cysteine Batch #10616125

Sodium fluoride (4.2026 g) was dissolved in 500 ml of water (obtained pH=9.01), and sterilized by filtration and placed in a sterile 1 liter Duran-Schott flask. Calcium chloride (11.1362 g) was dissolved in 500 ml of water (obtained pH=10.04) and N-Acetyl-Cysteine (16.3313 g) was added (obtained pH=1.63). Sodium hydroxide (solid pellets Merck product 1064981000 batch 60467298008) was added until pH 8.02 and sodium hydroxide 0.5N was added (5 ml) to reach pH 8.30 and this slightly violet solution (coloration appears above pH 5) was sterilized by filtration. The CaCl2-N-acetyl-cysteine solution was added to the NaF solution. Water washing were carried out according to Scheme 5. Supernatant pH: see Table 12.

### Ca/F/N-Acetyl-Cysteine Batch #11000101

Sodium fluoride (4.2101 g) was dissolved in 500 ml of water (obtained pH=9.35), and sterilized by filtration and placed in a sterile 1 liter Duran-Schott flask. Calcium chloride (11.1715 g) was dissolved in 500 ml of water (obtained pH=9.97) and N-Acetyl-Cysteine (16.3469 g) was added (obtained pH=1.79). Sodium hydroxide was added until pH 7.69 and sodium hydroxide 0.5N was added (5 ml) to reach pH 8.36 and this slightly violet solution (coloration appears above pH 5) was sterilized by filtration. The CaCl2-N-acetyl-cysteine solution was added to the NaF solution. Water washing were carried out according to Scheme 5. Supernatant pH is given in Table 12).

### Ca/F/Glutathione Batch #10616185

Sodium fluoride (4.2098 g) was dissolved in 500 ml of water (obtained pH=8.9), and sterilized by filtration and placed in a sterile 1 liter Duran-Schott flask. Calcium chloride (11.1025 g) was dissolved in 500 ml of water (obtained pH=10.00) and this solution filtered on 0.22 μm and replaced in a clean 800 ml Becker. Glutathione was added (obtained pH=2.67). Sodium hydroxide was added to reach pH 8.57 and this solution was sterilized by filtration. The CaCl2-Glutathione solution was added to the NaF solution. Water washing were carried out according to Scheme 5. Supernatant pH is given in Table 13.

TABLE 13
Precipitation in presence of Glutathione

| | Batch # | | | | | |
|---|---|---|---|---|---|---|
| | 11000033 | 11000030 | 11000086 | 11000099 | 10616185 | 11000194 |
| Starting | Ratio | Ratio | Ratio | Ratio | Ratio 1/1/1 | Ratio |
| pH | 1/1/1 | 1/1/0.1 | 1/1/0.1 | 1/1/0.1 | pH 8.9 8.5 | 1/1/0.1 |
| | pH 6.9 | pH 9.5 | pH 9.3 8.6 | pH 9.58 | | pH 9. 8.58 |
| | 7.04 | 8.6 | | 8.57 | | |
| Washing | pH | pH | pH | pH | pH | pH |
| W1 | 7.07 | 8.51 | 8.43 | 8.41 | 8.74 | ND |
| W2 | 7.15 | 8.66 | 8.56 | 8.54 | 8.79 | ND |
| W3 | 7.17 | 8.74 | 8.78 | 8.65 | 8.86 | 8.51 |
| W4 | 7.31 | 8.88 | 8.85 | 8.78 | 8.94 | 8.71 |
| W5 | 7.35 | 9.03 | 9.18 | 9.02 | 9.06 | 8.81 |
| W6 | 7.34 | 9.14 | 9.21 | 9.21 | 9.11 | 8.84 |
| W7 | 7.34 | 9.28 | 9.35 | 9.34 | 9.17 | 9.07 |
| W8 | 7.40 | 9.27 | 9.48 | 9.24 | 9.23 | 9.20 |
| W9 | 7.30 | 9.22 | 9.62 | 9.39 | 9.33 | 9.38 |
| W10 | 7.18 | 9.17 | 9.64 | 9.44 | 9.36 | 9.54 |
| Conc. | 23.52 mg/ml | 27.20 | 24.76 | 23.19 | 18.43 | 22.02 |
| Ca % | 45.8 43.3 | 46.2 39.5 | 45.6 44.0 | 46.6 47.0 | 39.7 37.5 | 43.9 35.5 |
| F % | | | | | | |
| E.C.P. | H₂O/KCl | H₂O/KCl | H₂O/KCl | H₂O/KCl | H₂O/KCl | H₂O/KCl |
| | 4.9 & 9.5 | 4.8 & 10.1 | 10.1 | 10.1 | 9.8 | 9.8 |
| | H₂O/KNO_{3:} | 9.2 | 10.1 | 10.4 | 9.1 | 9.1 |
| | 8.8 | | | | | |
| μgN/ml | 2526 | 2178 | 2789 | 2425.2 | 2062.4 | 2870 |
| μgN/mg | 105.0 | 78.5 | 112 | 98.56 | 109.51 | 130 |

### Ca/F/Glutathione Batch #11000030

Sodium fluoride (4.2260 g) was dissolved in 500 ml of water (obtained pH=9.47), and sterilized by filtration and placed in a sterile 1 liter Duran-Schott flask. Calcium chloride (11.120 g) was dissolved in 500 ml of water (obtained pH=9.92) and this solution filtered on 0.22 μm and replaced in a clean 800 ml Becker. Glutathione (3.0726 g) was added (obtained pH=2.72). Sodium hydroxide and sodium hydroxide 0.5M was added to reach pH 8.59 and this solution is sterilized by filtration. The CaCl2-Glutathione solution was added to the NaF solution. Water washing were carried out according to Scheme 5. Supernatant pH was given in Table 13.

### Ca/F/Glutathione Batch #11000033

Sodium fluoride (4.2097 g) was dissolved in 500 ml of water (obtained pH=9.35), HCl 0.3M was added to reach pH 6.94 and this solution sterilized by filtration and placed in a sterile 1 liter Duran-Schott flask.

Calcium chloride (11.117 g) was dissolved in 500 ml of water (obtained pH=9.91) and this solution filtered on 0.22 μm and replaced in a clean 800 ml Becker. Glutathione (30.7 g) was added (obtained pH=2.64). Sodium hydroxide and HCl 0.3M was added to reach pH 7.04 and this solution was sterilized by filtration. The CaCl2-Glutathione solution was added to the NaF solution. Water washing were carried out according to Scheme 5. Supernatant pH is given in Table 13.

### Ca/F/Glutathione Batch #11000086

Sodium fluoride (4.2235 g) was dissolved in 500 ml of water (obtained pH=9.34), and this solution sterilized by filtration and placed in a sterile 1 liter Duran-Schott flask. Calcium chloride (11.1431 g) was dissolved in 500 ml of water (obtained pH=9.92) and this solution filtered on 0.22 μm and replaced in a clean 800 ml Becker. Glutathione (3.0782 g) was added (obtained pH=2.84). Sodium hydroxide and sodium hydroxide 0.5M was added to reach pH 8.59 and this solution was sterilized by filtration. The CaCl2-Glutathione solution was added to the NaF solution. Water washing were carried out according to Scheme 5. Supernatant pH is given in Table 13.

### Ca/F/Glutathione Batch #11000099

Sodium fluoride (4.1101 g) was dissolved in 500 ml of water (obtained pH=9.58), and this solution sterilized by filtration and placed in a sterile 1 liter Duran-Schott flask. Calcium chloride (1101228 g) was dissolved in 500 ml of water and this solution filtered on 0.22 μm and replaced in a clean 800 ml Becker (obtained pH=10.05). Glutathione (3.0716 g) was added (obtained pH=2.79). Sodium hydroxide and sodium hydroxide 0.5M was added to reach pH 8.57 and this solution was sterilized by filtration. The CaCl2-Glutathione solution was added to the NaF solution. Water washing were carried out according to Scheme 5. Supernatant pH is given in Table 13.

### Ca/F/Glutathione Batch #11000194

Sodium fluoride (4.2059 g) was dissolved in 500 ml of water (obtained pH=9.24), and this solution sterilized by filtration and placed in a sterile 1 liter Duran-Schott flask. Calcium chloride (11.1371 g) was dissolved in 500 ml of water (obtained pH=9.99) and this solution filtered on 0.22 μm and replaced in a clean 800 ml Becker. Glutathione (3.0912 g) was added (obtained pH=3.07). Sodium hydroxide and sodium hydroxide 0.5M was added to reach pH 8.58 and this solution was sterilized by filtration. The CaCl2-Glutathione solution was added to the NaF solution. Water washing were carried out according to Scheme 5. Supernatant pH is given in Table 13.

### Ca/F/Glutathione Oxide Batch #10616198

Sodium fluoride (0.4220 g) was dissolved in 50 ml of water (obtained pH=8.64), and sterilized by filtration and placed in a sterile 100 ml Duran-Schott flask. Calcium chloride (1.1112 g) was dissolved in 30 ml of water (obtained pH=9.97) and Glutathione oxide (6.5611 g) was added (obtained pH=6.27). Sodium hydroxide 0.5N and 0.05M was added to reach pH 7.55 and this solution was sterilized by filtration. The CaCl2-Glutathione oxide solution was added to the NaF solution. Water washing were carried out according to Scheme 5. Supernatant pH is given in Table 14.

TABLE 14
Precipitation of Calcium fluoride composites and washing of CaPhosphate

| Precipitation in presence of Glutathione | | | | | | | |
|---|---|---|---|---|---|---|---|
| oxide (GSSG) or uric or folic acid | | | | | | CaPhosphate | |
| | F/Ca/GSSG ratio | | | | F/Ca/Folic | Brenntag | |
| | 0.01/0.01/0.01 | 0.1/0.1/0.005 | 0.1/0.1/0.01 | F/Ca/Uric | 1/1/0.001 | 2011-51 | |
| | 10616198 | 11000139 | 11000140 | 11000182 | 11481018 | 11000160 | |
| Starting pH | 8.64 7.55 | 9.82 8.21 | 9.82 8.12 | 8.60 6.24 | 6.98 9.73 | Scheme 1 | |
| Washing | pH | pH | pH | pH | pH | pH | Osm |
| W1 | 7.55 | 7.84 | 8.07 | 5.33 | 5.65 | 6.21 | 292 |
| W2 | 7.59 | 7.66 | 8.10 | 5.49 | 5.75 | 6.42 | 145 |
| W3 | 7.67 | 7.84 | 8.14 | 5.58 | 5.72 | 6.60 | 73 |
| W4 | 7.75 | 7.95 | 8.07 | 5.65 | 5.73 | 6.77 | 36 |
| W5 | 7.86 | 7.75 | 8.25 | 5.76 | 5.73 | 6.81 | 19 |
| W6 | 7.95 | 7.71 | 8.57 | 5.85 | 5.81 | 7.02 | 8 |
| W7 | 8.00 | 7.98 | 8.41 | 5.93 | 5.92 | 7.14 | 3 |
| W8 | 7.99 | 8.02 | 8.52 | 6.05 | 6.16 | 6.76 | 1 |
| W9 | 8.02 | 7.89 | 8.50 | 6.01 | 6.08 | 6.66 | 0 |
| W10 | 8.05 | 7.93 | 8.45 | 5.99 | 6.09 | 6.77 | 0 |

| | | | | | | |
|---|---|---|---|---|---|---|
| Conc. mg | 11.0 | 22.1 | 22.6 | 12.82 | 15.02 | 21.9 |
| dry/ml | | | | | | |
| Ca % F % | Not Done | 49.0 45.8 | 47.1 43.1 | 48.6 45.7 | 45.0 43.8 | Ca: 37.0% |
| E.C.P. KCl | 5.2 & 9.1 | 9.7 | 9.9 | No cross. | 4.8 | 7.3 |
| E.C.P. KNO₃ | 8.7 | 9.3 | 9.8 | 8.4 | 8.1 | 7.6 |
| μgN/ml | 2226 | 4130 | 4400 | 260.3 | 2034 | NA |
| μgN/mg | 179.1 | 183.5 | 190.7 | 19.69 | 135.4 | NA |

### Ca/F/Glutathione Oxide Batch #11000139

Sodium fluoride (4.20838 g) was dissolved in 50 ml of water (obtained pH=9.82), and sterilized by filtration and placed in a sterile 1 liter Duran-Schott flask. Calcium chloride (1.1528 g) was dissolved in 500 ml of water (obtained pH=9.97) and this solution filtered on 0.22 μm and replaced in a clean 800 ml Becker. Glutathione oxide (3.2255 g) was added (obtained pH=6.84). Sodium hydroxide and sodium hydroxide 0.5M was added to reach pH 8.21 and this solution was sterilized by filtration. The CaCl2-Glutathione oxide solution was added to the NaF solution. Water washing were carried out according to Scheme 5. Supernatant pH is given in Table 14.

### Ca/F/Glutathione Oxide Batch #11000140

Sodium fluoride (4.20027 g) was dissolved in 50 ml of water (obtained pH=9.82), and sterilized by filtration and placed in a sterile 1 liter Duran-Schott flask. Calcium chloride (1.1068 g) was dissolved in 500 ml of water (obtained pH=9.94) and this solution filtered on 0.22 μm and replaced in a clean 800 ml Becker. Glutathione oxide (6.56150 g) was added (obtained pH=6.55). Sodium hydroxide and sodium hydroxide 0.5M was added to reach pH 8.12 and this solution was sterilized by filtration. The CaCl2-Glutathione oxide solution was added to the NaF solution. Water washing were carried out according to Scheme 5. Supernatant pH is given in Table 14.

### Ca/F/Thiolactate Batch #11000031

Sodium fluoride (4.2078 g) was dissolved in 500 ml of water (obtained pH=9.56), and sterilized by filtration and placed in a sterile 1 liter Duran-Schott flask. Calcium chloride (11.1187 g) was dissolved in 500 ml of water (obtained pH=9.89) and this solution filtered on 0.22 μm and replaced in a clean 800 ml Becker. Thiolactic acid (8.5 ml was added (obtained pH=1.96). Sodium hydroxide was added to reach pH 9.54 and this solution was sterilized by filtration. The CaCl2-thiolactate solution was added to the NaF solution. Water washing were carried out according to Scheme 5. Supernatant pH is given in Table 15.

TABLE 15
Precipitation of in presence of Adipate or Thiolactate

| | Batch # | | | | | |
|---|---|---|---|---|---|---|
| | 11000129 | 11481026 | 11481027 | 11000059 | 11000060 | 11000031 |
| Starting pH | 8.02 10.81 | 5.94 9.82 | 7.18 9.82 | 7.16 6.97 | 7.93 8.07 | 9.56 9.54 |
| Washing | pH | pH | pH | pH | pH | pH |
| W1 | 7.97 | 5.56 | 6.93 | 6.17 | 7.93 | 9.73 |
| W2 | 7.75 | 5.66 | 6.95 | 6.28 | 8.13 | 9.85 |
| W3 | 7.58 | 5.72 | 6.94 | 6.07 | 8.09 | 10.00 |
| W4 | 7.50 | 5.72 | 6.84 | 6.04 | 8.51 | 10.12 |
| W5 | 7.58 | 5.99 | 6.92 | 5.87 | 8.73 | 10.23 |
| W6 | 7.08 | 6.06 | 6.79 | 5.86 | 8.96 | 10.36 |
| W7 | 6.65 | 6.20 | 6.78 | 5.91 | 9.23 | 10.43 |
| W8 | 6.52 | 6.28 | 6.72 | 5.96 | 9.49 | 10.42 |
| W9 | 6.68 | 6.39 | 6.63 | 6.02 | 9.68 | 10.52 |
| W10 | 6.77 | 6.37 | 6.56 | 6.04 | 9.85 | 10.56 |
| Conc. | 29.9 mg/ml | 26.32 | 31.61 | 10.6 | 19.36 | 23.88 |
| Ca % F % | 50.0 | 46.3 | 45.1 | 50.1 | 49.5 | 47.2 |
| | 44.4 | 42.2 | 44.7 | 43.2 | 46.1 | 42.7 |
| E.C.P. | 6.6 | 7.2 | 6.7 | 9.5? & | 5.5 & 8.4 | 5.4 & 9.5 |
| H₂O/KCl | | | | 10.2 | | |
| E.C.P. | 6.6 | 7.9 | 7.5 | 6.1 | 7.1 | 6.7 (9.5?) |
| KNO₃ | | | | | | |
| % by | 4.4% | 4.10% | 5.27% | 0.9% | 1.8% | 6% |
| Titration | | | | | | |

### Ca/F/Thiolactate Batch #11000059

Sodium fluoride (4.2146 g) was dissolved in 500 ml of water (obtained pH=9.47), and HCl 0.03N as added to reach pH 7.16. This solution was sterilized by filtration and placed in a sterile 1 liter Duran-Schott flask. Calcium chloride (11.1780 g) was dissolved in 500 ml of water (obtained pH=9.98) and this solution filtered on 0.22 μm and replaced in a clean 800 ml Becker. Thiolactic acid (8.5 ml) was added (obtained pH=2.05). Sodium hydroxide and NaOH 0.03M was added to reach pH 7.16 and this solution was sterilized by filtration. The CaCl2-thiolactate solution was added to the NaF solution. Water washing were carried out according to Scheme 5. Supernatant pH is given in Table 15.

### Ca/F/Thiolactate Batch #11000060

Sodium fluoride (4.2161 g) was dissolved in 500 ml of water (obtained pH=9.45), and HCl 0.03N as added to reach pH 7.93. This solution was sterilized by filtration and placed in a sterile 1 liter Duran-Schott flask. Calcium chloride (11.1786 g) was dissolved in 500 ml of water (obtained pH=9.96) and this solution filtered on 0.22 μm and replaced in a clean 800 ml Becker. Thiolactic acid (8.5 ml was added (obtained pH=1.96). Sodium and NaOH 0.03M was added to reach pH 8.07 and this solution was sterilized by filtration. The CaCl2-thiolactate solution was added to the NaF solution. Water washing were carried out according to Scheme 5. Supernatant pH was given in Table 15.

### Ca/F/Adipic Acid Batch #11000129

Adipic acid (7.3070 g) was dissolved in 500 ml of water (obtained pH=2.81). Sodium hydroxide was added to reach pH5.39. Sodium fluoride (4.1967 g) was added; the pH at this stage was 5.59. More NaOH was added to reach pH 8.02, and this solution was sterilized by filtration and placed in a sterile 1 liter Duran-Schott flask.

Calcium chloride (11.1481 g) was dissolved in 500 ml of water (obtained pH=10.81). This solution was sterilized by filtration and poured into NaF/Adipate sterile solution. Water washing were carried out according to Scheme 6. Supernatant pH is given in Table 15).

### Ca/F/Adipic Acid Batch #11481026

Adipic acid (7.3174 g) was dissolved in 500 ml of water (obtained pH=2.99). Sodium hydroxide was added to reach pH5.11. Sodium fluoride (4.1979 g) was added; the pH at this stage was 5.48. More NaOH was added to reach pH 5.94, and this solution was sterilized by filtration and placed in a sterile 1 liter Duran-Schott flask.

Calcium chloride (11.1163 g) was dissolved in 500 ml of water (obtained pH=9.82). This solution was sterilized by filtration and poured into NaF/Adipate sterile solution. Water washing were carried out according to Scheme 6. Supernatant pH is given in Table 15.

### Ca/F/Adipic Acid Batch #11481027

Adipic acid (7.3151 g) was dissolved in 500 ml of water (obtained pH=2.98). Sodium was added to reach pH5.36. Sodium fluoride (4.1995 g) was added; the pH at this stage was 5.48. More NaOH was added to reach pH 7.18, and this solution was sterilized by filtration and placed in a sterile 1 liter Duran-Schott flask.

Calcium chloride (11.1209 g) was dissolved in 500 ml of water (obtained pH=9.82). This solution was sterilized by filtration and poured into NaF/Adipate sterile solution. Water washing were carried out according to Scheme 6. Supernatant pH is given in Table 15.

### Ca/F/Uric Acid Batch #11000182

Sodium fluoride (4.1650 g) was dissolved in 500 ml of water (obtained pH=9.52). Uric acid (0.13223 g) was added by several small portions allowing time (overnight) for dissolution and compensate for pH drops by NaOH (0.5M) addition when needed; pH at this stage being 8.60. This solution was sterilized by filtration and placed in a sterile 1 liter Duran-Schott flask. Calcium chloride hexahydrate (21.9929 g) was dissolved in 500 ml of water (obtained pH=6.24). This solution was sterilized by filtration and poured into NaF/Urate sterile solution. Water washing were carried out according to Scheme 6. Supernatant pH: see Table 14.

### Ca/F/Folic Acid (Vitamin M) Batch #11481018

Sodium fluoride (4.2026 g) was dissolved in 500 ml of water (obtained pH=9.33). Folic acid (0.4481 g) was added and pH drops to 5.74. NaOH 0.5M was added to reach pH 6.98 and this solution was sterilized by filtration and placed in a sterile 1 liter Duran-Schott flask. Calcium chloride (11.1183 g) was dissolved in 500 ml of water (obtained pH=9.73). This solution was sterilized by filtration and poured into NaF/Folate sterile solution. Water washing were carried out according to Scheme 6. Supernatant pH: see Table 14.

### Ca/F/Hypoxanthine Batch 11481198

Sodium fluoride (4.21 g) was dissolved in 500 ml of water, 1.36 g of hypoxanthine was added, NaOH 0.5M was added to reach pH 9.83 and this solution was sterilized by filtration and placed in a sterile 1 liter Duran-Schott flask. Calcium chloride dihydrate (14.73 g) was dissolved in 500 ml of water and 1 ml of NaOH 0.05M was added to reach pH 9.31 and this solution was sterilized by filtration and poured into NaF/hypoxanthine sterile solution. Water washing were carried out according to Scheme 6. Supernatant pH see Table 15A.

### Ca/F/Xanthine Batch 11481199

Sodium fluoride (4.22 g) was dissolved in 500 ml of water, 1.52 g of Xanthine was added, NaOH 0.5M was added to reach pH 10.75 and this solution was sterilized by filtration and placed in a sterile 1 liter Duran-Schott flask. Calcium chloride dihydrate (14.71 g) was dissolved in 500 ml of water and 1 ml of NaOH 0.05M was added to reach pH 9.39 and this solution was sterilized by filtration and poured into NaF/Xanthine sterile solution. Water washing were carried out according to Scheme 6. Supernatant pH see Table 15A.

### Ca/F/Guanine Batch 11481195

To 1.52 g of Guanine in 50 ml of water NaOH (solid pellets) (1.4 g) were added. After dissolution 4.21 g of Sodium fluoride and 450 ml of water were added. The pH of the solution was 12.52. This solution was sterilized by filtration and placed in a sterile 1 liter Duran-Schott flask. Calcium chloride dihydrate (14.71 g) was dissolved in 500 ml of water and 0.5 ml of NaOH 0.05M was added to reach pH 8.48 and this solution was sterilized by filtration and poured into NaF/guanine sterile solution. Water washing were carried out according to Scheme 6. Supernatant pH see Table 15A.

### Ca/F/Cytosine Batch 11954009

Sodium fluoride (4.22 g) was dissolved in 500 ml of water, 1.10 g of cytosine was added (obtained pH 9.03) and this solution was sterilized by filtration and placed in a sterile 1 liter Duran-Schott flask. Calcium chloride dihydrate (14.73 g) was dissolved in 500 ml of water and 1 ml of NaOH 0.05M was added to reach pH 9.18 and this solution was sterilized by filtration and poured into NaF/Cytosine sterile solution. Water washing were carried out according to Scheme 6. Supernatant pH see Table 15A.

### Ca/F/Thymine Batch 11954064

Sodium fluoride (4.22 g) was dissolved in 500 ml of water, 1.26 g of hypoxanthine was added, NaOH 0.5M was added to reach pH 9.11 and this solution was sterilized by filtration and placed in a sterile 1 liter Duran-Schott flask. Calcium chloride dihydrate (14.75 g) was dissolved in 500 ml of water and 0.5 ml of NaOH 0.05M was added to reach pH 8.31 and this solution was sterilized by filtration and poured into NaF/Thymine sterile solution. Water washing were carried out according to Scheme 6. Supernatant pH see Table 15A.

TABLE 15A
Precipitation in presence of various organic materials

| | Ca/F/ | | Ca/F/ | | Ca/F/ | | Ca/F/ | | Ca/F/ | |
|---|---|---|---|---|---|---|---|---|---|---|
| | Guanine | | Hypoxanthine | | Xanthine | | Cytosine | | Thymine | |
| Batch# | 11481195 | | 11481198 | | 11481199 | | 11954009 | | 11954064 | |
| Starting pH | 12.52 | 8.48 | 9.83 | 9.31 | 10.75 | 9.39 | 9.03 | 9.18 | 9.11 | 8.31 |
| Washing | pH | | pH | | pH | | pH | | pH | |
| W1 | 11.66 | | 9.12 | | 9.05 | | 6.92 | | 8.66 | |
| W2 | 11.55 | | 9.21 | | 9.15 | | 6.68 | | 8.82 | |
| W3 | 11.54 | | 9.26 | | 9.28 | | 6.12 | | 8.86 | |
| W4 | 11.51 | | 9.46 | | 9.31 | | 6.15 | | 8.75 | |
| W5 | 11.50 | | 9.50 | | 8.48 | | 5.94 | | 8.23 | |
| W6 | 11.42 | | 9.62 | | 9.27 | | 5.99 | | 6.52 | |
| W7 | 11.32 | | 9.68 | | 9.49 | | 6.29 | | 6.37 | |
| W8 | 11.29 | | 9.67 | | 8.62 | | 5.90 | | 6.35 | |
| W9 | 11.29 | | 9.60 | | 9.52 | | 5.81 | | 6.63 | |
| W10 | 11.19 | | 9.56 | | 9.63 | | 5.82 | | 6.66 | |
| Conc. | 21.5 | | 13.09 | | 20.3 | | 13.74 | | 9.8 | |
| Ca % F % | 44.2 | 38.3 | 48.0 | 44.0 | 48.7 | 44.3 | 49.8 | 45.3 | 43.7 | 45.8 |

### CaAdipate Batch 11954096

Calcium carbonate (5 g) was suspended in 500 ml of water. Adipic acid (7.3 g) was added. Additional water quantities were added until the volume reaches 750 ml and the mix was heated at 60° C. during 1 hour. This solution was sterilized by filtration (0.22 μm filter). The resulting solution heated and concentrated by evaporation until about 280 ml total volume. Crystals were separated from the supernatant and dried at 80° C. during 5 days. This product is used as Calcium adipate reference during thermogravimetry measurements.

### Analysis of Composite Organic Content by Thermogravimetry.

Dry material sample were submitted to thermogravimetry. Weight losses from RT to 600° C. under N₂ and from 600° C. to 800° C. under O₂ were recorded and represent by difference the quantity of burned organic material.

TABLE 15B
Results of thermogravimetry analyses

| | | | | | | | Re- |
|---|---|---|---|---|---|---|---|
| | Between | | | | Between | | sidual |
| | 0 and | | Between | | 600 et | | material |
| Weight losses/ | 200° C. | | 200 et 600° C. | | 800° C. | | at |
| samples | under N2 | | under N2 | | under Air | | 800° C. |
| 11000123 | 0.5% | | 0.9% | | 0.1% | | 98.5% |
| CaF₂ (without | | | | | | | |
| organic) | | | | | | | |
| | | Δ | | Δ | | Δ | |
| Thymine | 100% | | | | | | |
| 11954064 | 0.7% | 0.2% | 1.1% | 0.2% | 0.1% | 0% | 98.1% |
| Ca/F/Thymine | | | | | | | |
| Adipic acid | 100% | | | | | | |
| 11954096 | 6.9% | | 42.5% | | 22.3% | | 28.3% |
| Ca-Adipate | | | | | | | |
| 11000129 | 1.5% | 1% | 7.5% | 6.6% | 3.0% | 2.9% | 88.0% |
| Ca/F/Adipate | | | | | | | |
| 11000060 | 1.1% | 0.6% | 2.4% | 1.5% | 0.3% | 0.2% | 96.2% |
| Ca/F/ | | | | | | | |
| Thiolactate | | | | | | | |
| 11000083 | 2.3% | 1.8% | 2.0% | 1.1% | 2.1% | 2.0% | 93.6% |
| Ca/F/CO₃ | | | | | | | |
| 11000099 | 2.3% | 1.8% | 5.7% | 4.8% | 2.1% | 2.0% | 89.9% |
| Ca/F/ | | | | | | | |
| Glutathion | | | | | | | |
| 11000140 | 3.2% | 2.7% | 13.3% | 12.4% | 4.8% | 4.7% | 78.7% |
| Ca/F/ | | | | | | | |
| Glutathione | | | | | | | |
| oxide | | | | | | | |
| 11481018 | 2.4% | 1.9% | 6.3% | 5.4% | 3.5% | 3.4% | 87.8% |
| Ca/F/Folic | | | | | | | |
| 11481064 | 1.9% | 1.4% | 3.1% | 2.2% | 0.2% | 0.1% | 94.8% |
| Ca/F/Cysteine | | | | | | | |
| 11481133 | 1.5% | 1.0% | 2.4% | 1.5% | 0.3% | 0.2% | 95.8% |
| Ca/F/N—Ac- | | | | | | | |
| Cysteine | | | | | | | |
| Uric acid | 0.1% | | 74.6% | | 25.3% | | |
| 11481186 | 1.4% | 0.9% | 1.9% | 1.0% | 0.4% | 0.3% | 96.3% |
| Ca/F/Uric | | | | | | | |
| 11481195 | 2.2% | 1.7% | 5.0% | 4.1% | 2.5% | 2.4% | 90.3% |
| Ca/F/Guanine | | | | | | | |
| Hypoxanthine | 0.2% | | 50.6% | | 49.2% | | |
| 11481198 | 1.2% | 0.7% | 2.0% | 1.1% | 0.5% | 0.4% | 96.3% |
| Ca/F/ | | | | | | | |
| Hypoxanthine | | | | | | | |
| Xanthine | 0.3% | | 67.7% | | 32.0% | | |
| 11481199 | 2.5% | 2.0% | 3.8% | 2.9% | 1.4% | 1.3% | 92.3% |
| Ca/F/Xanthine | | | | | | | |
| 11954009 | 0.5% | 0% | 1.0% | 0.1% | 0.1% | 0% | 98.4% |
| Ca/F/Cytosine | | | | | | | |

### Example 3A

### Adsorption of MPL

Adsorption of MPL (nanoparticles in water) on various inorganic particles were carried out under aseptic conditions and % of MPL adsorption measured by STEP® technology (space- and time-resolved extinction profile) using LumiSizer® instrument. Table 16 summarizes those adsorptions data and shows that presence of the vaterite type of carbonate in the calcium-fluoride-carbonate composite allows 100% adsorption of 100 μg MPL on 500 μg inorganic composite in 1 ml water. Ca/F/N-Acetyl-cysteine (batch 10616125) gives similar results.

TABLE 16
Estimated % of adsorption of 1169.6 μg MPL on various carriers quantities
based on the remaining supernatant % of transmitted light

| | Carrier weights | | | | | |
|---|---|---|---|---|---|---|
| | 1721 μg | 3442 μg | 6884 μg | 11703 μg | 23407 μg | |
| | 100 μg MPL/μg carrier/ml | | | | | % CaCO₃ |
| | μg carrier/ml | | | | | by |
| | 125 | 250 | 500 | 1000 | 2000 | titration |
| CaF₂ (#9440194) t = 0 | ND | ND | 20% | 66% | 100% | 0 |
| CaF₂ (#9440194) t = 5 days | ND | ND | 66% | ND | ND | NA |
| Ca/F/CO₃ (#9923124) t = 0 | ND | ND | 66% | 100% | 100% | 2.9 |
| Vaterite/Calcite ratio: | | | | | | |
| high | | | | | | |
| Ca/F/CO₃ (#9923124) | ND | ND | 100% | ND | ND | ND |
| t = 5 days | | | | | | |
| Vaterite/Calcite ratio: | | | | | | |
| high | | | | | | |
| Ca/F/CO₃ (#8833157) t = 0 | ND | ND | 90% | 80% | ND | 4.5 |
| CO₃⁻⁻/F⁻/Ca⁺⁺:1/38/20 | | | | | | |
| Vaterite/Calcite ratio: | | | | | | |
| high | | | | | | |
| Ca/F/CO₃ (#8833156) t = 0 | 66% | 70% | 75% | 80% | ND | 11.4 |
| CO₃⁻⁻/F⁻/Ca⁺⁺:2/36/20 | | | | | | |
| Vaterite/Calcite ratio: | | | | | | |
| high | | | | | | |
| Ca/F/CO₃ (#8833152) t = 0 | ND | ND | 0% | 0% | ND | 86 |
| CO₃⁻⁻/F⁻/Ca⁺⁺:18/04/20 | | | | | | |
| Vaterite/Calcite ratio: low | | | | | | |
| Ca/F/Cys. (#9440197) t = 0 | ND | ND | 70% | 100% | ND | NA |
| Ca/F/Cys. (#9440197) t = 5 | ND | ND | 70% | 100% | ND | NA |
| days | | | | | | |
| Ca/F/NACys. (#10616125) | ND | ND | 70% | 100% | ND | NA |
| t = 0 | | | | | | |
| Ca/F/NACys. (#10616125) | ND | ND | 100% | 100% | ND | NA |
| t = 5 d | | | | | | |

### Example 3B

### Stability and Thermostability of Adsorbed Antigen

Improvement of stability and thermostability of adsorbed antigen was demonstrated with F4T, a rather instable antigen. Table 17 summarizes the antigen adsorption conditions and final composition.

TABLE 17
Formulation conditions of F4T proteins for stability studies

| Inorganic | CaF₂ | | Ca/F/Cysteine | | Ca/F/CO₃ | |
|---|---|---|---|---|---|---|
| 3 mg dry material/ml | #8833190 | | #9440099 | | #8833172C | |
| Antigen | 225 μg/ml | | | | | |
| Buffer | Tris 10 mM, Phosphate 2.5 mM, | | | | | |
| | NaCl 5 mM, Sorbitol 4.7% | | | | | |
| pH | pH 7.5 | | pH 8.0 | | pH 8.0 | |
| Immunostimulant* | − | + | − | + | − | + |

*Immunostimulant was of the GSK liposome family.

Antigens adsorptions were determined by HPLC quantification carried out on the supernatant after centrifugation (to discard the antigen/inorganic part). No antigen in the supernatant was interpreted as equal to 100% of adsorbed antigen. Stability of adsorption was measured at t=0 and at t=1 month 4° C., while thermo-stability was determined after 1 month 30° C. (Table 18).

TABLE 18
Adsorption of F4T measured by HPLC-RP

| | | T = 1 | T = 1 |
|---|---|---|---|
| | T = 0 | month 4° C. | month 30° C. |
| F4T + CaF₂ | 90 | 80 | 88 |
| F4T + CaF₂ + Liposome | 92 | 86 | 90 |
| F4T + Ca/F/Cysteine | 100 | 94 | 93 |
| F4T + Ca/F/Cysteine + | 100 | 95 | 93 |
| liposome | | | |
| F4T + Ca/F/CO₃ | 93 | 85 | 91 |
| F4T + Ca/F/CO₃ + liposome | 94 | 89 | 91 |

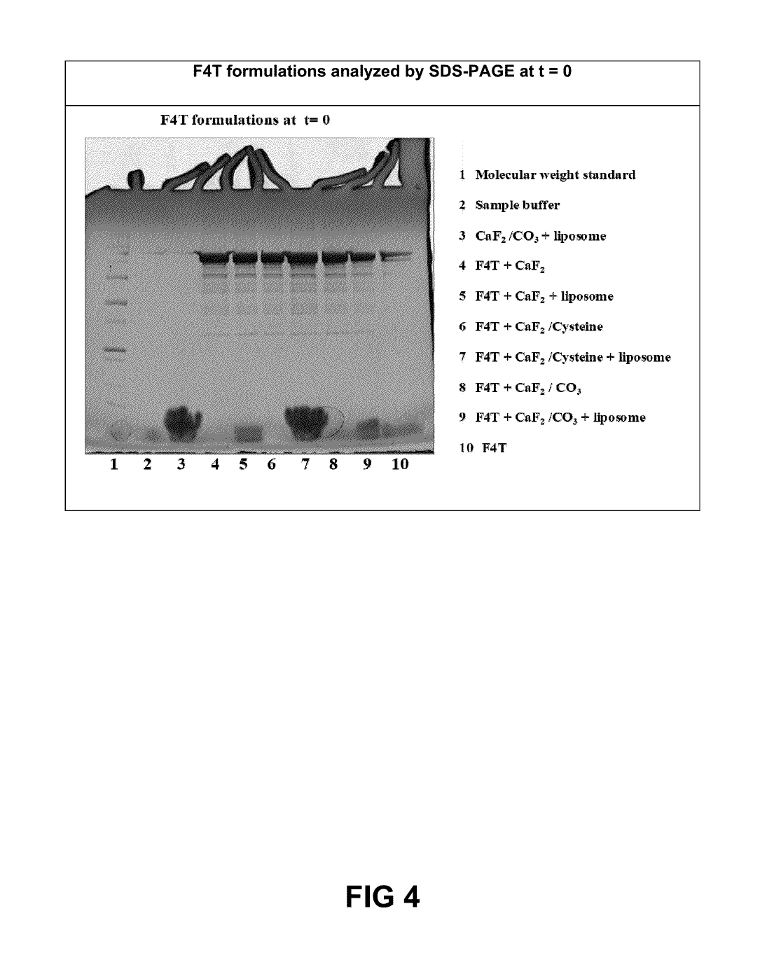

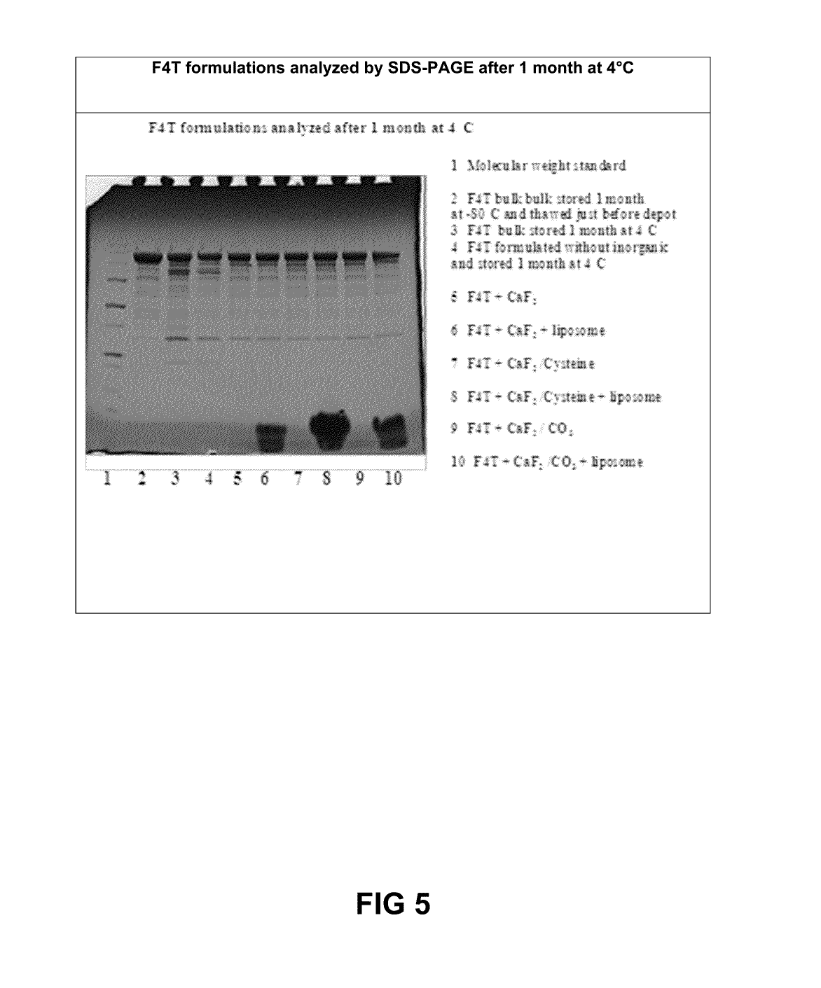

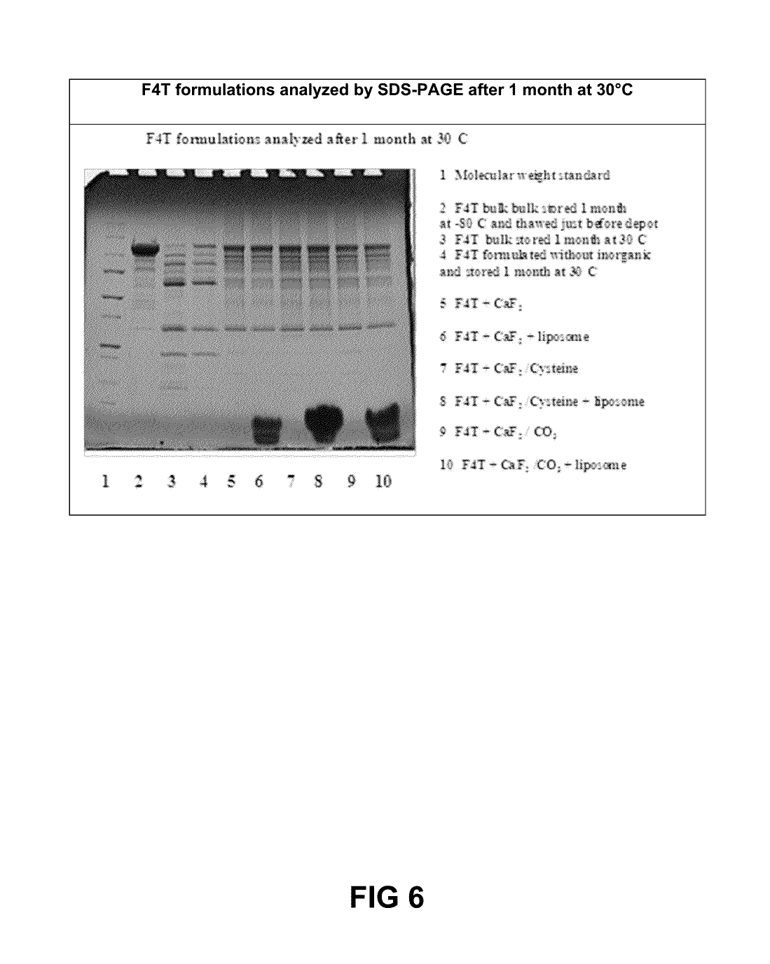

Stability of F4T antigens profiles evaluations were carried out by SDS-PAGE analyses at t=0 (FIG. 4) and compared at t=1 month 4° C. (FIG. 5), while thermo-stability was determined after 1 month at 30° C. (FIG. 6). Being adsorbed by electrostatic forces, the antigen was released by the SDS-PAGE gel environment and applied experimental migration conditions. In all cases of this stability and thermo-stability measurements, there were impressive conservation of the antigens profile of the adsorbed material compared to none adsorbed control samples.

### Example 4

### Immune Response to ClfA_{N123} and HepB Antigens

Immune Response of Composite-ClfA_{N123} adsorbed antigen

Adsorption of composite-ClfA_{N123} was presented in Table 19 and formulations compositions were presented in Table 20.

TABLE 19
Adsorption of composite-ClfA_{N123}
measured by HPLC-SEC

| | | T = 1 | T = 1 |
|---|---|---|---|
| | T = 0 | month 4° C. | month 37° C. |
| CompositeClfA_{N123} + CaF₂ | 90 | 100 | 94 |
| CompositeClfA_{N123} + | 100 | 100 | 100 |
| Ca/F/Cysteine | | | |
| CompositeClfA_{N123} + | 91 | 100 | 100 |
| Ca/F/CO₃ | | | |

TABLE 20
Formulation of compositeClfA_{N123} for animal studies

| | | Composite | Buffer | Immuno- | Sor- | mOsmoles/ |
|---|---|---|---|---|---|---|
| Groups | | ClfA_{N123} | pH 6 | stimulant | bitol | kg |
| None | — | 60 μg/ml | Maleate | Emulsion | 4.7% | 319 |
| adsorbed | | | 10 mM | | | |
| Ca/F/CO₃ | 1 mg/ | 60 μg/ml | Maleate | Emulsion | 4.7% | 310 |
| #9440195 | ml | | 10 mM | | | |
| Ca/F/N- | 1 mg/ | 45 μg/ml | Maleate | Emulsion | 3.5% | 235 |
| Acetyl- | ml | | 7.5 mM | | | |
| Cysteine | | | | | | |
| #9440196 | | | | | | |
| CaF₂ | 1 mg/ | 60 μg/ml | Maleate | Emulsion | 4.7% | 314 |
| #8833190 | ml | | 10 mM | | | |
| Ca/F/ | 1 mg/ | 60 μg/ml | Maleate | Emulsion | 4.7% | 308 |
| Cysteine | ml | | 10 mM | | | |
| #9440197 | | | | | | |

As shown (FIG. 7) the immunogenicity of the antigen was maintained when the antigen was adsorbed on the different carriers.

### Immune Response of HepB Adsorbed Antigen

Formulations compositions are presented (Table 21).

TABLE 21
Animal antibodies obtained with Hepb (20 μg) adsorbed on inorganic
carrier (1240 μg) and possible correlation with organic
content of Calcium fluoride composites

| | | | | Organic |
|---|---|---|---|---|
| | | Antibody | | content |
| | | GMT +/− | | μmoles/ |
| Formulations | Batch # | IC95 | Performance | mg dry |
| Engerix (AIOOH) | A13AFO0024 | 6208 | Reference | NA |
| Ca/F/Glutathione | 11000099 | 6165 | equivalent | 2.27 |
| Ca/F/Glutathione | 11000140 | 5850 | | 2.34 |
| oxide | | | | |
| Ca/F/CO₃ | 11000083 | 5742 | | 1.02 |
| Ca/F/adipate | 11000129 | 4247 | Statistically | 0.30 |
| Ca/F/N-Acetyl- | 11000101 | 4188 | not different | 0.56 |
| Cysteine | | | | |
| Ca/F/Thiolactate | 11000060 | 3298 | Lower | 0.17 |
| Ca/F/OH | 11000123 | 3078 | | NA |
| Ca Phosphate | 11000160 | 1475 | | NA |

Animal experience LIMS 20130115, all formulated in 10 mM TRIS pH 6.8 and sorbitol 4.7% except for Engerix which was in phosphate buffer and 150 mM NaCl. CaPhosphate from Brenntag water washed according to Scheme 1 and Table 14.

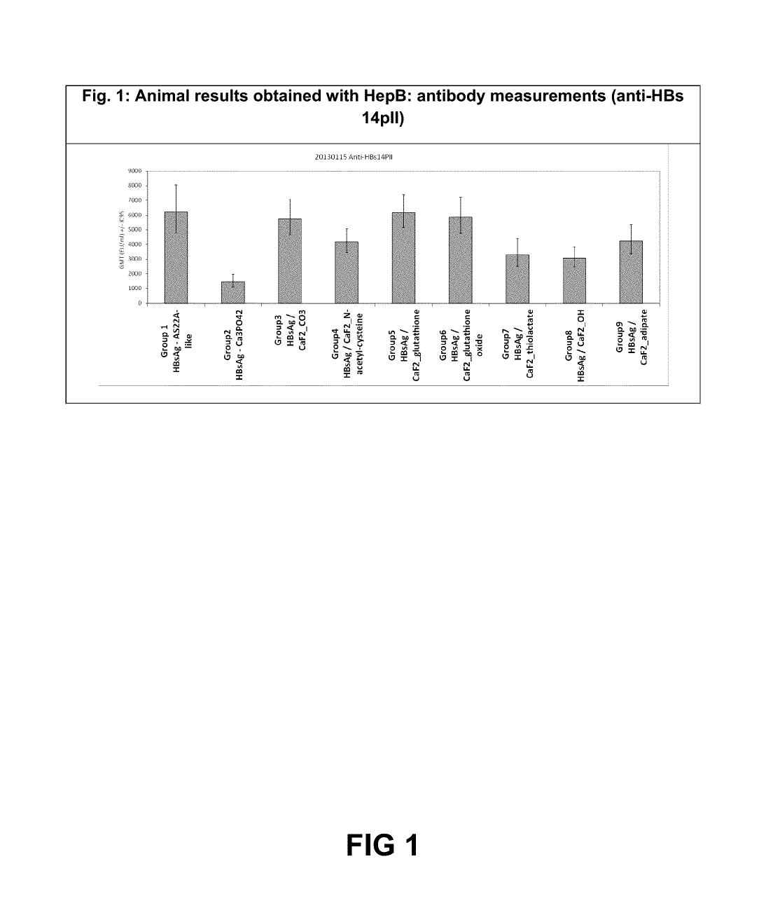

As shown, the antibody measurements (anti-HBs 14pII) (FIG. 1) responses of the antigen were maintained when the antigen was adsorbed on the different carriers of the CaF₂ family described herein.

### Example 5

### Immune Response

### Immune Response of HepB Adsorbed Antigen

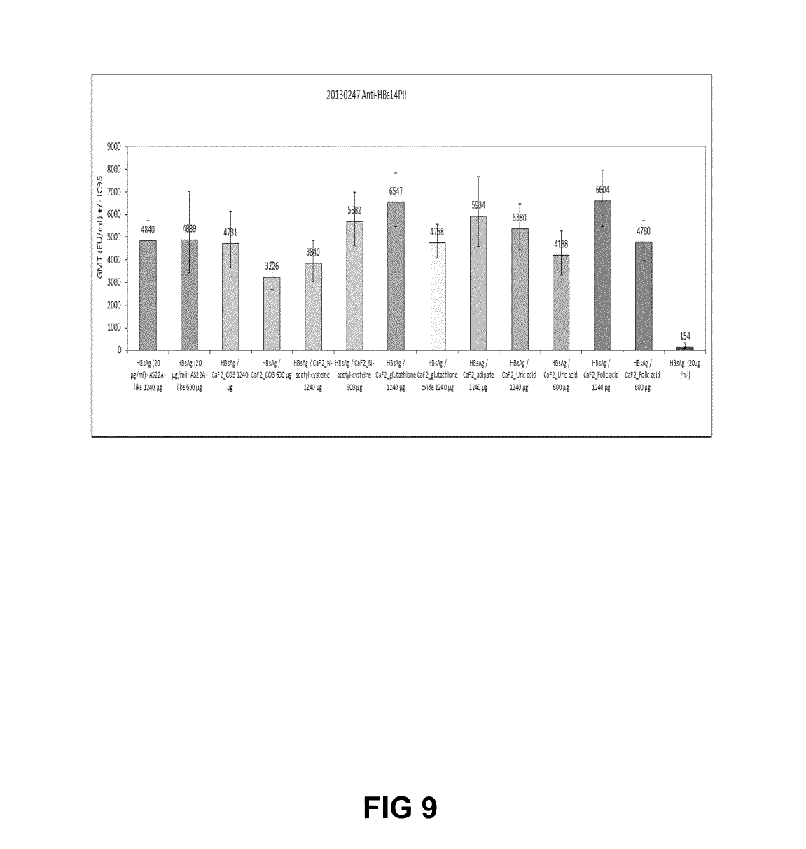

For this investigation of calcium fluoride composites in vivo, five calcium fluoride composites and AlOOH were selected from Example 4 for repetition using the same calcium fluoride composite batches as used in the Example 4; in addition HepB adsorbed on ½ initial calcium fluoride composite quantity was selected for investigation (1240 mg versus 600 mg calcium fluoride composite). Further, previously untried calcium fluoride composites (containing a Z different from that of the previously tested batches) were selected for this investigation. The composites tested were as follows:
1° AlOOH 1240 μg
2° AlOOH 600 μg
3° CaF2/CO₃1240 μg
4° CaF2/CO₃600 μg
5° CaF2N-AcetylCysteine 1240 μg
6° CaF2N-AcetylCysteine 600 μg
7° CaF2/Glutathione 1240 μg
8° CaF2/Glutathione oxide 1240 μg
9° CaF2/Adipate 1240 μg
10° CaF2/Uric acid 1240 μg (new calcium fluoride composite)
11° CaF2/Uric acid 600 μg (new calcium fluoride composite)
12° CaF2/folic acid 1240 μg (new calcium fluoride composite)
13° CaF2/folic acid 600 μg (new calcium fluoride composite)
14° HepB only (no carrier)
   Preliminary results (without statistical treatment) are summarized in FIG. 9 in the same order from top (#1=“Engerix-like” formula) to (#14 hepB alone).

### Example 6

### Immunization of Balb/C Mice with Recombinant F Protein Adsorbed on Different Composites Induces Similar Levels of RSV Neutralizing and rF Binding Antibodies than Alum-Hydroxide

Adsorption measurements with composite were conducted with a recombinant RSV F protein (rF): five rF-composite formulations were selected for immunogenicity testing in Balb/C mice, in comparison with Alum hydroxide—or Calcium Phosphate-adsorbed rF (see Table 22).

TABLE 22
experimental design of the mouse immunogenicity study

| | ANTIGEN | | ADJUVANT |
|---|---|---|---|
| | (RF) DOSE | | DOSE |
| GROUP | (μG/ANIMAL) | ADJUVANT | (μG/ANIMAL) |
| 1 | 2 | Alum hydroxide | 50 |
| 2 | 0.1 | Alum hydroxide | 50 |
| 3 | 2 | CaF2/Adipate | 60 |
| 4 | 0.1 | CaF2/Adipate | 60 |
| 5 | 2 | CaF2/Cysteine | 60 |
| 6 | 0.1 | CaF2/Cysteine | 60 |
| 7 | 2 | CaF2/N-acetyl-cysteine | 60 |
| 8 | 0.1 | CaF2/N-acetyl-cysteine | 60 |
| 9 | 2 | CaF2/Uric acid | 60 |
| 10 | 0.1 | CaF2/Uric acid | 60 |
| 11 | 2 | CaF2/Thiolactate | 60 |
| 12 | 0.1 | CaF2/ Thiolactate | 60 |
| 13 | 2 | CaPhosphate | 60 |
| 14 | 0.1 | CaPhosphate | 60 |

Groups of Balb/c mice (n=9/group) were immunized intra-muscularly twice at a 3-week interval with the formulations of Table 22. The rF antigen was used at two different doses with each of the selected adjuvants.

Sera from all mice were individually collected on Day 35 (14 days after the second immunization) and tested for the presence of RSV neutralizing antibodies using a plaque reduction assay and for the anti-rF IgG concentration by ELISA. For the neutralization assay, serial dilutions of each serum were pre-incubated for 20 min with RSV A (Long strain) at 33° C. After incubation, the virus-serum mixture was transferred to plates previously seeded with Vero cells. On each plate, cells in one column were incubated with virus only (100% infectivity) and 2 wells received no virus or serum (cell controls). Plates were incubated for 2 hours at 33° C., medium was removed and RSV medium containing 0.5% CMC (low viscosity carboxymethylcellulose) was added to all wells. The plates were incubated for 3 days at 33° C. before immunofluorescence staining.

For ELISA, the statistical methods employed to compare different groups were Analysis of Variances (ANOVA 1 or ANOVA 2) on the log 10 values.

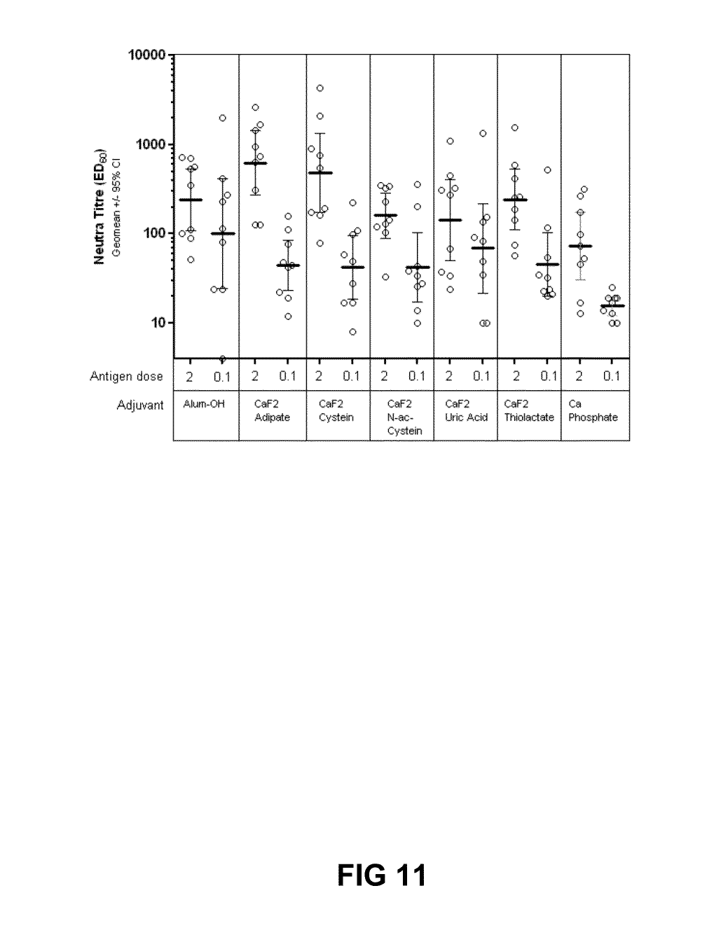

Results presented in FIG. 11 indicated that no significant difference could be observed between the neutralizing antibody response induced by any of the composites and alum hydroxide, at the two doses of antigen tested. In addition, at the 2 μg antigen dose, composite adipate induced significantly higher neutralizing antibody titers than the composite N-acetyl-cysteine and the composite uric acid. Calcium Phosphate was the less immunogenic adjuvant as it induced significantly lower neutralizing antibody titers than alum hydroxide (0.1 μg rF), composite adipate (2 μg rF), composite cysteine (2 μg rF) and composite uric acid (0.1 μg rF).

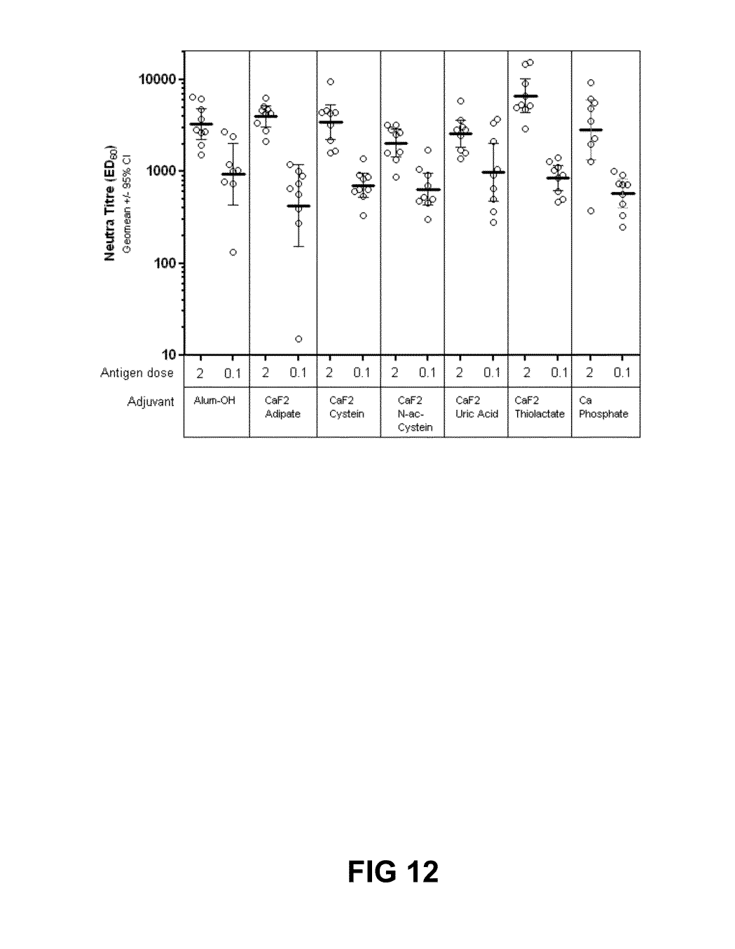

Very similar results were obtained when serum samples were tested for concentrations of anti-rF IgG (FIG. 12).

### Example 7

### Immunization of Balb/C Mice with Recombinant F Protein Adsorbed on Different Composites is Able to Significantly Reduce RSV Viral Load in Lungs Following RSV Challenge

Based on the experiment described in Example 6, composite adipate, composite Cysteine and composite uric acid were selected for a mouse efficacy study, in comparison with Alum hydroxide. The experimental design is described in Table 23.

TABLE 23
experimental design of the mouse efficacy study

| | ANTIGEN (RF) DOSE | | ADJUVANT DOSE |
|---|---|---|---|
| GROUP | (μG/ANIMAL) | ADJUVANT | (μG/ANIMAL) |
| 1 | 2 | Alum hydroxide | 50 |
| 2 | 2 | CaF2/Adipate | 50 |
| 3 | 2 | CaF2/Cysteine | 60 |
| 4 | 2 | CaF2/Uric Acid | 60 |
| 5 | 0 (PBS group) | None | — |

Groups of Balb/c mice (n=8/group) were immunized intra-muscularly twice at a 3-week interval with the formulations of Table 23. Fourteen days after the second immunization, animals were challenged intra-nasally with 1.54×10⁶ pfu of RSV. To measure the efficacy of these exemplary vaccines, lungs were harvested 4 days post RSV challenge and individually weighed and homogenized. Serial dilutions (8 replicates each) of each lung homogenate were incubated with Vero cells and wells containing plaques were identified by immunofluorescence, 6 days after seeding. The viral titer was determined using the Spearman-Kärber method for TCID50 calculation and was expressed per gram of lung. The statistical method employed to compare different groups was an ANOVA 1 on the log 10 values.

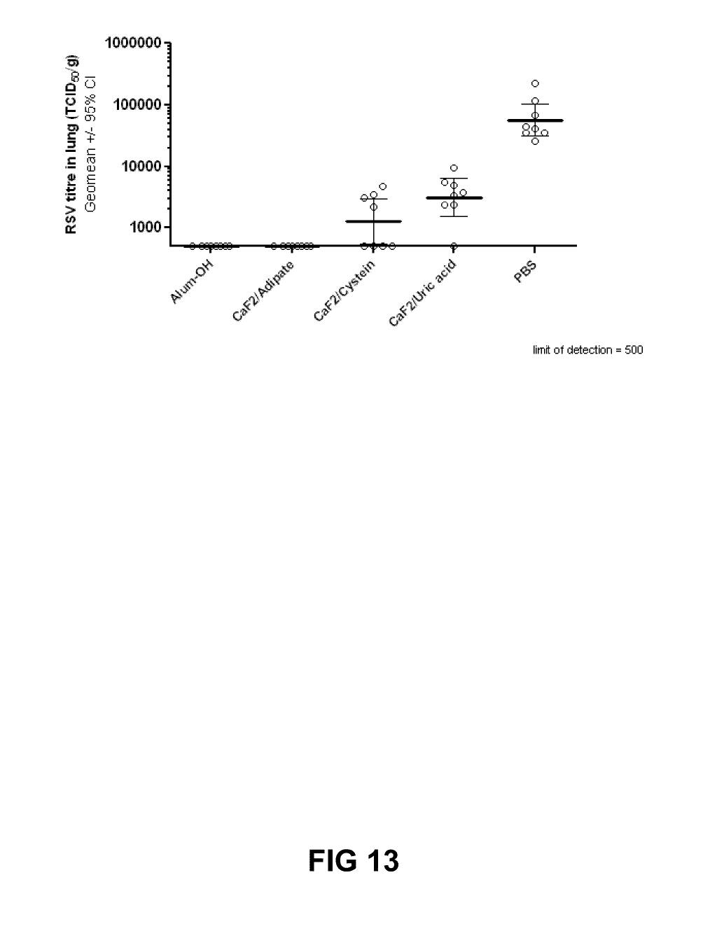

Results presented in FIG. 13 indicated that vaccination with 2 μg rF+composite Adipate was the only composite formulation able to completely abolish RSV replication in mouse lungs, as was vaccination with 2 μg rF+alum-OH. The two other composites tested (composite/Cysteine and composite/Uric Acid) did not completely prevent viral replication but significantly (P<0.001) reduced viral replication in the lungs. These results show that despite similar antibody responses, different composites can induce qualitatively different vaccine responses, as shown by the different degrees of inhibition of viral replication in RSV challenged Balb/c mice.

### Example 8

### Evaluation of Composite 19F-DT Formulations in the Balb/C Mouse Immunogenicity Model

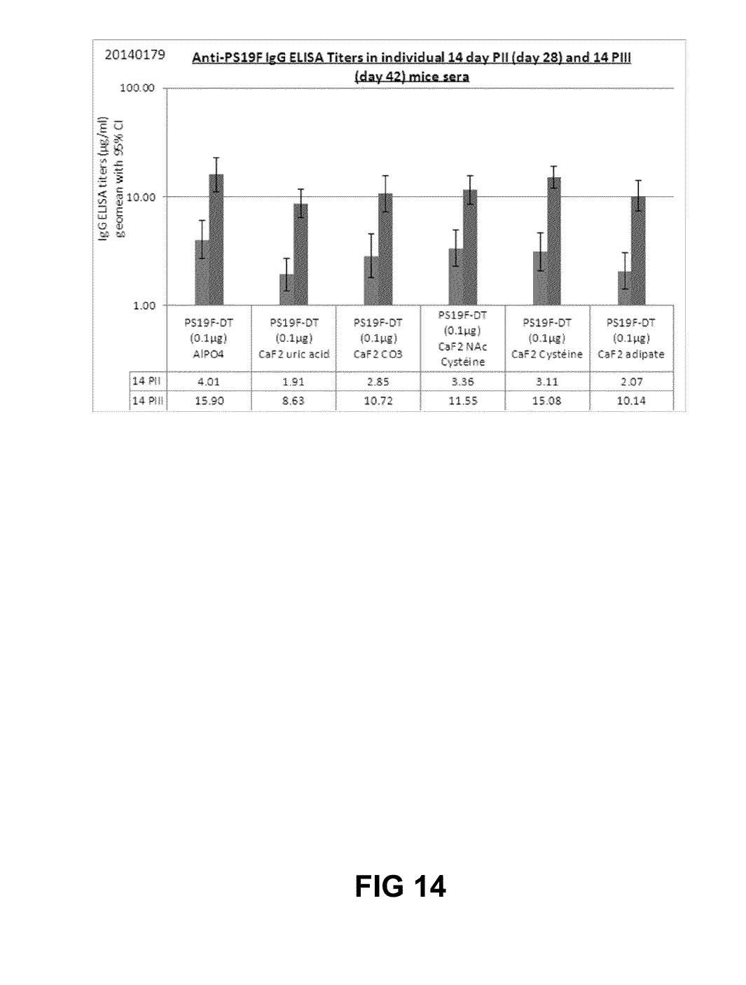

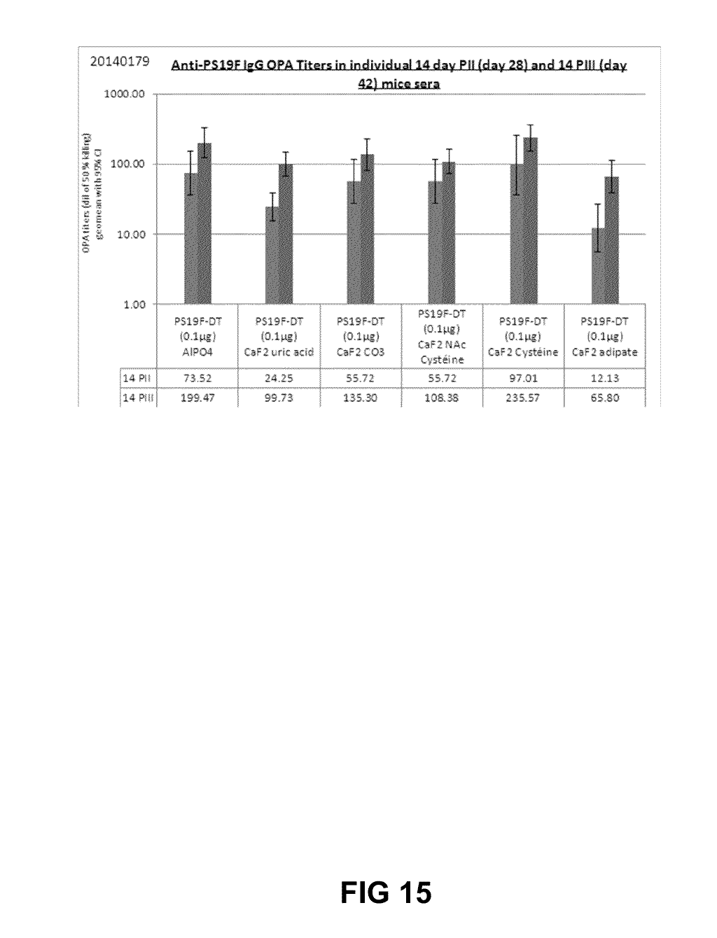

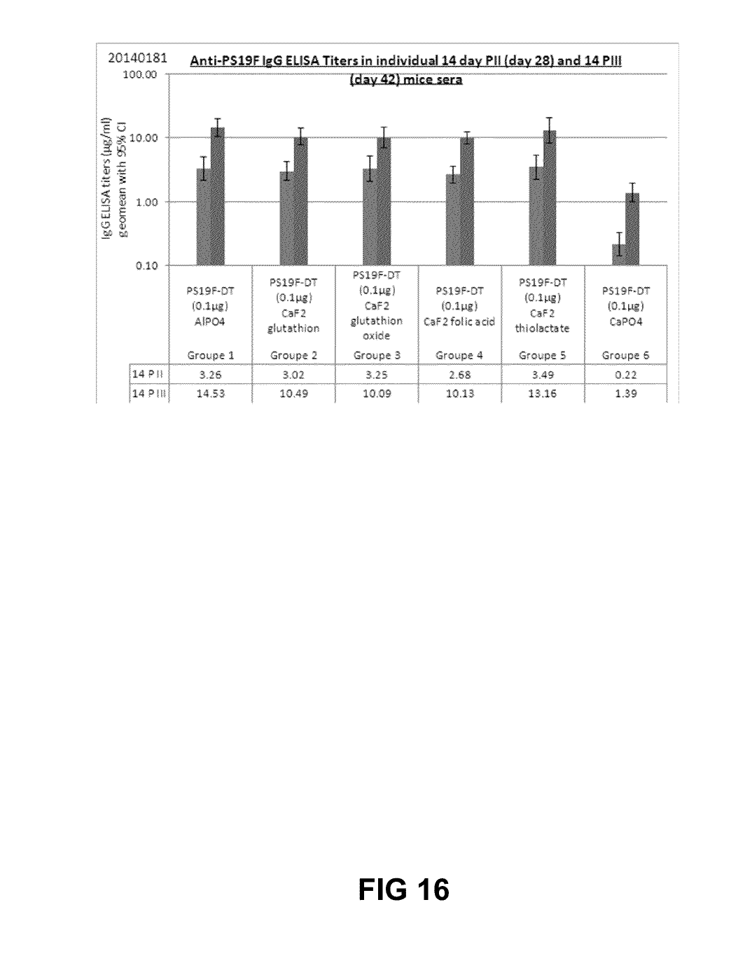

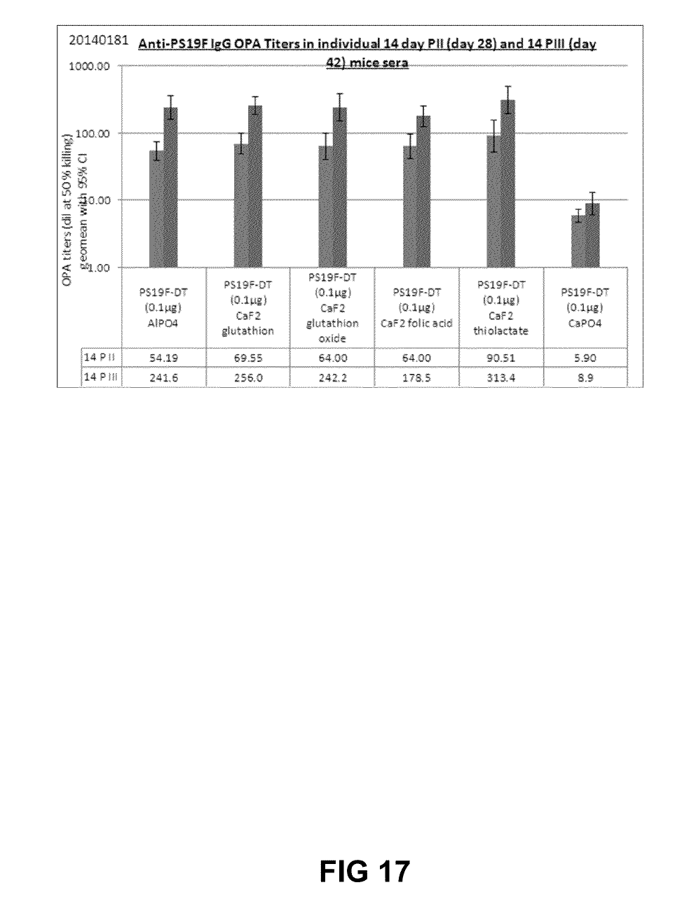

Ten different 19F-DT formulations were tested in the Balb/c mouse immunogenicity model through two experiments. Composite formulations were intramuscularly administered at the dose of 0.1 μg 19F-DT at days 0, 14 and 28. IgG levels and OPA titers were determined in individual sera collected at days 28 (ELISA and OPA except for the experience 20140179 made on pooled sera) and 42 (ELISA and OPA). All candidates except the Ca/F/Uric acid and Ca₃(PO4)₂ (used as a control) formulations reached the non-inferiority criteria versus AlPO4. See FIGS. 14-17.

### Example 9

### Animal Results of Composite PRN

Briefly, groups of BALB/c mice (n=16/group) were immunized intramuscularly twice with a 2-week interval Sera from all mice were individually collected, fourteen days after the first immunization and seven days after the second immunization and tested for the presence of anti-PRN IgG antibodies according to the following protocol.

96-well plates were coated with PRN (6 μg/ml) in a carbonate-bicarbonate buffer (50 mM) and incubated overnight at 4° C. After the saturation step with the PBS-BSA 1% buffer, mouse sera were diluted at 1/100 in PBS-BSA 0.2% Tween 0.05% and serially diluted in the wells from the plates (12 dilutions, step ½). An anti-mouse IgG coupled to the peroxidase was added (1/5000 dilution). Colorimetric reaction was observed after the addition of the peroxidase substrate (OPDA), and stopped with HCL 1M before reading by spectrophotometry (wavelengths: 490-620 nm). For each serum tested and standard added on each plate, a 4-parameter logistic curve was fit to the relationship between the OD and the dilution (Softmaxpro). This allowed the derivation of each sample titer expressed in STD titers

TABLE 24
PRN antigen with various composites.

| | GMT +/− | GMT +/− | upper- | GMT- | upper- | |
|---|---|---|---|---|---|---|
| | IC95 | IC95 | GMT | lower | GMT | GMT |
| PRN (16 μg/ml) | 14PI | 7PII | 14PI | 14PI | 7PII | 7PII |
| PRN-AlOOH-like | 101 | 8878 | 65 | 40 | 7599 | 4094 |
| Composite (160 μg dry) | | | | | | |
| PRN-AlOOH-like DTPa | 185 | 14945 | 56 | 43 | 6222 | 4393 |
| (248 μg dry) | | | | | | |
| PRN-CaF2-CO3 | 196 | 19110 | 45 | 36 | 9909 | 6525 |
| PRN-CaF2-N-Acetyl- | 164 | 16669 | 49 | 38 | 7230 | 5043 |
| Cysteine | | | | | | |
| PRN-CaF2-Cysteine | 190 | 13661 | 50 | 39 | 5433 | 3887 |
| PRN-CaF2-Glutathione | 186 | 16978 | 59 | 45 | 7261 | 5086 |
| PRN-CaF2-Thiolactate | 128 | 9073 | 43 | 32 | 5616 | 3469 |
| PRN-CaF2-Folic acid | 165 | 4743 | 113 | 67 | 4180 | 2222 |
| PRN-CaF2-Adipate | 368 | 11104 | 83 | 68 | 3979 | 2929 |
| PRN-CaF2-Guanine | 129 | 16285 | 39 | 30 | 6535 | 4664 |
| PRN-CaF2- | 114 | 9608 | 48 | 34 | 10802 | 5085 |
| Hypoxanthine | | | | | | |
| PRN-CaF2-Xanthine | 153 | 19444 | 46 | 36 | 9107 | 6202 |
| PRN-CaF2-Uric acid | 182 | 15306 | 40 | 33 | 5017 | 3779 |

### Example 10

### Denge/Composite

Denge-4 formulated in 4.7% sorbitol in TRIS buffer at pH 8.0 was adsorbed on different composite to reach a final concentration of 4 μg antigen per ml. After centrifugation, antigen was measured in the supernatant by ELISA. The 100% ELISA value is given to similar Denge-4 formulation measured after centrifugation. Thus, low ELISA values indicate high adsorption of the antigen on composite. Table 25 indicates the composite quantities involved in each formulations.

TABLE 25
Adsorption of DENGE-4 on various composite

| | | | | | % |
|---|---|---|---|---|---|
| | | Composite | Found | | recovery |
| | | quantities | Denge-4 | | relative |
| | | (μg dry | ELISA | % | (/plain |
| Composite | Batch# | material) | concentrations | recovery | centri) |
| Denge-4 | No | 0 | 1.435 | 35.9 | 100.0 |
| | Composite | | | | |
| Ca/F/Cysteine | 10616183 | 300 | <0.05 | <1.3 | <3.5 |
| Ca/F/Adipate | 11000129 | 150 | <0.05 | <1.3 | <3.5 |
| Ca/F/CO₃ | 11000083 | 300 | <0.05 | <1.3 | <3.5 |
| Ca/F/N-Ac | 11481133 | 300 | <0.05 | <1.3 | <3.5 |
| Cysteine | | | | | |
| Ca/F/Xanthine | 11481199 | 150 | <0.05 | <1.3 | <3.5 |
| Ca/F/Uric | 11481186 | 300 | <0.05 | <1.3 | <3.5 |
| Ca/F/Guanine | 11481195 | 150 | <0.05 | <1.3 | <3.5 |

### Example 11

### Adsorption of QS21

The immunostimulant QS21 is too hemolytic to be used in human vaccine by itself. Several formulations containing cholesterol avoid this problem. A hemolytic test are mandatory for new formulations involving quilliac extracts and derivatives (C. Kensil et al., 1991, J. Immunology, vol 146 (2) p. 431-437) (S. Soltysik et al. Vaccine 1995 vol 13 (15) p. 1403-1410) (B. Rönnberg et al., 1995, Vaccine, vol 13 (14) p.1375-1382) (B. P. da Silva et al. 2005, Vaccine vol 23 p 1061-1070 and Oliveira-Freitas et al., 2006, Vaccine, vol 24 p 3909-3920 and C. Rodrigues et al., 2012, Chemistry and Biodiversity, vol 9 p. 58-67).

It was found that adsorption of QS21 on inorganic composites prevents hemolysis of chicken red blood cells without addition of cholesterol on the formulation. This was supported by the results obtained using Ca/F/Glutathione at 50, 25 and 12.5 μg QS21 adsorbed on 6.7 mg inorganic composite (batch 11000099) (Table 26).

TABLE 26
Results of Hemolytic test for Ca/F/Glutathione

| | Carrier | μg QS21/ml | | | | |
|---|---|---|---|---|---|---|
| | 11000099 | 100 μg | 50 μg | 25 μg | 12.5 μg | 0 μg |
| Delta | 6.7 mg/ml | 1.072 | 0.017 | −0.006 | −0.006 | 0 |
| DO | | | | | | |
| Delta | No carrier | 1.811 | 1.806 | 1.811 | 1.756 | 0 |
| DO | | | | | | |
| % | | 59 | 0.9 | −0.3 | −0.3 | 0 |
| Status | | Fail | Pass | Pass | Pass | Pass |

1-25. (canceled)

26. An adjuvant composition comprising a calcium fluoride composite, said composite comprising Ca, F, and Z, wherein Z is glutathione, further comprising an immunologically active saponin fraction.

27. The adjuvant composition of claim 26,
wherein Z is glutathione,
and wherein the composite comprises between 51% Ca, 48% F, no more than 1% glutathione (w/w) and 28% Ca, 20% F, 52% glutathione (w/w).

28. The adjuvant composition of claim 26, wherein the composition is pharmaceutically acceptable.

29. The adjuvant composition of claim 26, wherein the calcium fluoride composite is in particulate form.

30. The adjuvant composition of claim 26, wherein the composite particles are in the nanoparticles or microparticles size domain.

31. The adjuvant composition of claim 26, further comprising a second composite wherein Z is not glutathione.

32. The adjuvant composition of claim 26 comprising an antigen, wherein the antigen is adsorbed to a calcium fluoride composite.

33. A method for reducing the hemolytic activity of QS21 comprising combining the steps of combining QS21 with a calcium fluoride composite.

34. A method for inducing an immunogenic response in a mammal in need thereof, said method comprising administering to said mammal an effective amount of an adjuvant composition.


SEQUENCE LISTING (6 sequences)

SEQ ID NO: 1 - DNA, 20 bp, Artificial Sequence
  Synthetic immunostimulatory oligonucleotide
tccatgacgttcctgacgtt

SEQ ID NO: 2 - DNA, 18 bp, Artificial sequence
  Synthetic immunostimulatory oligonucleotide
tctcccagcgtgcgccat

SEQ ID NO: 3 - DNA, 30 bp, Artificial sequence
  Synthetic immunostimulatory oligonucleotide
accgatgacgtcgccggtgacggcaccacg

SEQ ID NO: 4 - DNA, 24 bp, Artificial sequence
  Synthetic immunostimulatory oligonucleotide
tcgtcgttttgtcgttttgtcgtt

SEQ ID NO: 5 - DNA, 20 bp, Artificial sequence
  Synthetic immunostimulatory oligonucleotide
tccatgacgttcctgatgct

SEQ ID NO: 6 - DNA, 22 bp, Artificial sequence
  Synthetic immunostimulatory oligonucleotide
tcgacgttttcggcgcgcgccg